(19) United States
(12) Patent Application Publication
(10) Pub. No.: US 2025/0280727 A1
(43) Pub. Date: Sep. 4, 2025

(54) Organic Compound And Organic Light-Emitting Diode Including Same

(71) Applicant: P&H TECH Co., Ltd, Gyeonggi-do (KR)

(72) Inventors: Jong-Kwan BIN, Gyeonggi-do (KR); Seung Jae LEE, Gyeonggi-do (KR)

(73) Assignee: P&H TECH Co., Ltd, Gyeonggi-do (KR)

(21) Appl. No.: 18/862,160

(22) PCT Filed: May 3, 2023
(86) PCT No.: PCT/KR2023/006020

§ 371 Date: Oct. 31, 2024

(30) Foreign Application Priority Data
May 4, 2022 (KR) 10-2022-0055186

(51) Int. Cl. H10K 85/60; C07D 209/86; C07D 405/12; C07D 405/14; C07D 409/12; H10K 50/18

(52) U.S. Cl. CPC H10K 85/636; C07D 209/86; C07D 405/12; C07D 405/14; C07D 409/12; H10K 85/633; H10K 50/181; H10K 85/615; H10K 85/626; H10K 85/6572; H10K 85/6574; H10K 85/6576

(57) ABSTRACT

The present invention relates to an organic compound used in organic layers, such as electron-blocking layers and hole transport layers, within an organic light-emitting diode, and an organic light-emitting diode that adopts the organic compound and display significantly improved device characteristics such as low-voltage operation, longevity, and luminous efficiency. The organic compound according to the present invention exhibits excellent hole injection and transport performance, high capability to confine triplet excitons, superior electron-blocking performance, and excellent stability in thin-film states. The organic light-emitting diode employing the compound in its organic layers, such as electron-blocking and hole transport layers, demonstrate notably superior characteristics in terms of low-voltage operation, longevity, and luminous efficiency compared to conventional devices and thus can be advantageously used in various illumination devices and display devices.

### TECHNICAL FIELD

The present invention relates to an organic compound, and more particularly, to an organic compound that is employed to organic layers such as a hole transport layer, an electron blocking layer, etc. provided in an organic light emitting device and an organic light emitting device that employs the same, thus achieving greatly improved luminescent properties such as low-voltage driving of the device and excellent luminous efficiency.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and red, and thus has recently become a subject of intense interest as a next-generation display device.

The organic light-emitting device is a self-emitting device in which electrons injected from an electron injection electrode (cathode electrode) and holes injected from a hole injection electrode (anode electrode) combine in a light-emitting layer to form excitons, which then emit light while releasing energy, and the organic light-emitting device as described above has the advantages of low driving voltage, high luminance, wide viewing angle, and fast response speed, and can be applied to full-color flat panel light-emitting displays, and are therefore in the spotlight as a next-generation light source.

However, in order for such an organic light-emitting device to exhibit the aforementioned characteristics, the structure of the organic layers in the device needs to be optimized, and the materials constituting each organic layer, such as a hole injection material, a hole transport material, a light-emitting material, an electron transport material, an electron injection material, and an electron blocking material, need to be supported by stable and efficient materials, but, there is still a need to continue developing the structures and materials of organic layers of a stable and efficient organic light-emitting device.

In particular, in order to implement higher light-emitting efficiency, there is a need for developing a material capable of being more suitably used not only as a hole injecting layer or a hole transport layer but also as an electron blocking layer by increasing the hole mobility.

### DISCLOSURE

### Technical Problem

An aspect of the present invention intends to provide a novel organic compound that is employed to organic layers such as a hole transport layer, an electron blocking layer, etc. in an organic light-emitting device to implement excellent luminescent properties such as low-voltage driving of the device, improved luminous efficiency, etc., and an organic light-emitting device including the same.

### Technical Solution

An aspect of the present invention provides an organic compound represented by Formula I below and an organic light-emitting device in which the organic compound is included in the device.

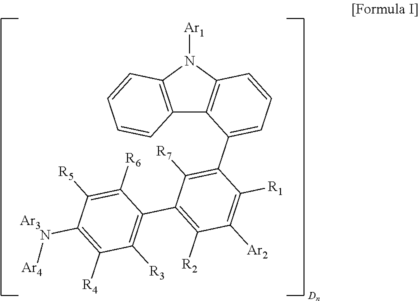

The characteristic structure of Formula I above and the definitions of compounds implemented thereby, R₁ to R₇, Ar₁ to Ar₄, D (deuterium), and n will be described below.

### Advantageous Effects

Since the organic compound according to the present invention has excellent hole injection and transport ability, excellent triplet exciton confinement ability, excellent electron blocking ability, and excellent stability in a thin film state, an organic light-emitting device using the organic compound in an organic layer such as an electron blocking layer or a hole transport layer has significantly excellent device characteristics such as low-voltage driving, long service life characteristics, and light-emitting efficiency compared to conventional devices, and can be usefully used in various lighting devices and display devices.

### BEST MODE

Hereinafter, the present invention will be described in more detail.

The present invention relates to an organic compound represented by the following [Formula I], which is characterized by structurally introducing an amine derivative into position 4 of carbazole using a substituted or unsubstituted biphenyl group as a linking group, and when the organic compound is used in various organic layers in an organic light-emitting device, preferably an electron blocking layer, a hole transport layer, and the like, it is possible to implement an organic light-emitting device with significantly improved device characteristics such as low-voltage driving, longevity, and luminous efficiency.

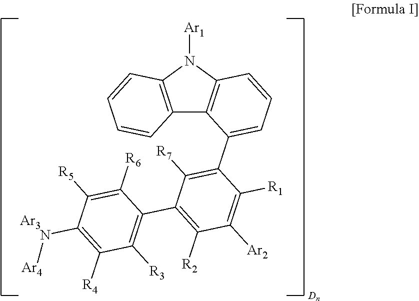

In [Formula I] above,

Ar₁ is selected from a substituted or unsubstituted aryl group having 6 to 30 carbon atoms and a substituted or unsubstituted heteroaryl group having 2 to 30 carbon atoms.

Ar₂ is selected from hydrogen, deuterium, a substituted or unsubstituted aryl group having 6 to 30 carbon atoms, or a substituted or unsubstituted heteroaryl group having 2 to 30 carbon atoms (provided that a carbazole group is excluded).

R₁ to R₇ are the same as or different from each other, and are each independently hydrogen or deuterium.

Ar₃ and Ar₄ are the same as or different from each other, and are each independently selected from a substituted or unsubstituted aryl group having 6 to 30 carbon atoms and a substituted or unsubstituted heteroaryl group having 2 to 30 carbon atoms.

D is deuterium, n means the number of hydrogen atoms in [Formula I] above, which are replaced with deuterium atoms (D), and n is an integer from 0 to 60.

[Formula I] above is characterized by being a compound in which not only the backbone structure but also R₁ to R₇ and Ar₁ to Ar₄ introduced therein are partially substituted with deuterium (D), and according to an exemplary embodiment of the present invention, the deuterium (D) substitution rate may be 10 to 90%.

As described above, the compound according to the present invention may be a compound containing at least one deuterium atom by replacing some hydrogen atoms in the [Formula I] structure with deuterium, and thus makes it possible to implement an organic light-emitting device having longevity by compensating for the short service life disadvantage of an organic light emitting device confirmed by a conventional moiety structure.

In addition, the compound represented by [Formula I] according to the present invention has a structural feature in a biphenyl group, which corresponds to a linking group linking a carbazole structure and an amine group, and is characterized in that Ar₂ introduced into the biphenyl group is an aryl group or a heteroaryl group (provided that the carbazole group is excluded), and in this case, at least one of Ar₂ and R₁ to R₇ introduced into the biphenyl group may be deuterium.

Meanwhile, in the definitions of Ar₁ to Ar₄ above, the ‘substituted or unsubstituted’ means substitution of Ar₁ to Ar₄ above with one or at least two substituents selected from the group consisting of deuterium, a cyano group, a halogen group, a hydroxy group, a nitro group, an alkyl group, a halogenated alkyl group, a deuterated alkyl group, an alkoxy group, a halogenated alkoxy group, a deuterated alkoxy group, a cycloalkyl group, a heterocycloalkyl group, an aryl group, a heteroaryl group, and a silyl group, substitution with a substituent to which two or more of the substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents of deuterium etc. In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents of deuterium etc.

In an embodiment of the present invention, examples of the substituents will be described in detail below, but are not limited thereto.

In the present invention, the alkyl group may be linear or branched, and although not particularly limited thereto, the number of carbon atoms is preferably 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In the present invention, the alkoxy group may be linear or branched, and although not particularly limited thereto, the number of carbon atoms is preferably 1 to 20, which is within a range that does not cause steric hindrance. Specific examples thereof include a methoxy group, an ethoxy group, an n-propoxy group, an isopropoxy group, an i-propyloxy group, an n-butoxy group, an isobutoxy group, a tert-butoxy group, a sec-butoxy group, an n-pentyloxy group, an neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group, and the like, but are not limited thereto.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In addition, in an embodiment of the present invention, the fluorenyl groups refer to structures in which two cyclic organic compounds are linked through one atom, and examples thereof include

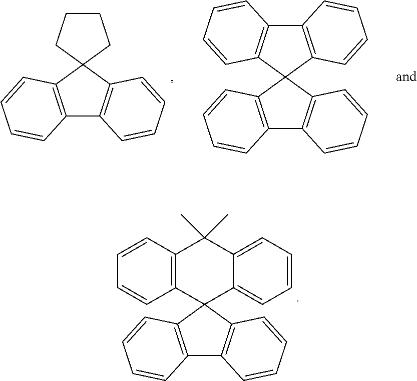

In an embodiment of the present invention, the fluorenyl groups include open structures in which one of the two cyclic organic compounds linked through one atom is cleaved, and examples thereof include and

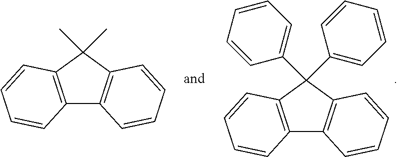

In addition, carbon atoms of the ring may be substituted with any one or more heteroatoms selected from among N, S and O, and examples thereof include

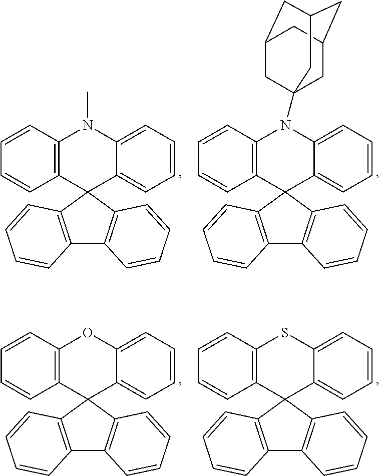

and the like.

In addition, carbon atoms of the ring may be substituted with any one or more heteroatoms selected from among N, S and O, and examples thereof include

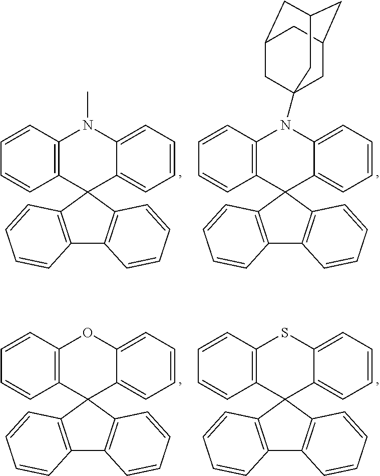

and the like.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (Cl), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

In the present invention, the amine group may be —NH₂, an alkylamine group, an arylamine group, an arylheteroarylamine group, etc., the arylamine group refers to amine substituted with an aryl group, the alkylamine group refers to amine substituted with an alkyl group, and the arylheteroarylamine group refers to amine substituted with aryl and heteroaryl groups. Examples of the arylamine group include a substituted or unsubstituted monoarylamine group, a substituted or unsubstituted diaryl amine group, or a substituted or unsubstituted triarylamine group. The aryl group and the heteroaryl group in the arylamine group and the arylheteroarylamine group may be a monocyclic aryl group, a monocyclic heteroaryl group, a polycyclic aryl group, or a polycyclic heteroaryl group, and the arylamine group and the arylheteroarylamine group including two or more aryl groups and heteroaryl groups may include a monocyclic aryl group (heteroaryl group), a polycyclic aryl group (heteroaryl group), or both a monocyclic aryl group (heteroaryl group) and a polycyclic aryl group (heteroaryl group).

In addition, the aryl group and the heteroaryl group in the arylamine group and the arylheteroarylamine group may be selected from examples of the above-mentioned aryl group and heteroaryl group.

Furthermore, various specific examples of the substituent according to the present invention can be clearly confirmed in the specific compounds described below.

The organic compound according to the present invention represented by [Formula I] above may be used as an organic layer of an organic light-emitting device due to its structural specificity as described above, and more specifically, may be used as a material for an electron blocking layer, a hole transport layer, and the like of the organic layer depending on the characteristics of various substituents to be introduced.

Preferred specific examples of the organic compound represented by Formula I according to the present invention include the following compounds but are not limited thereto.

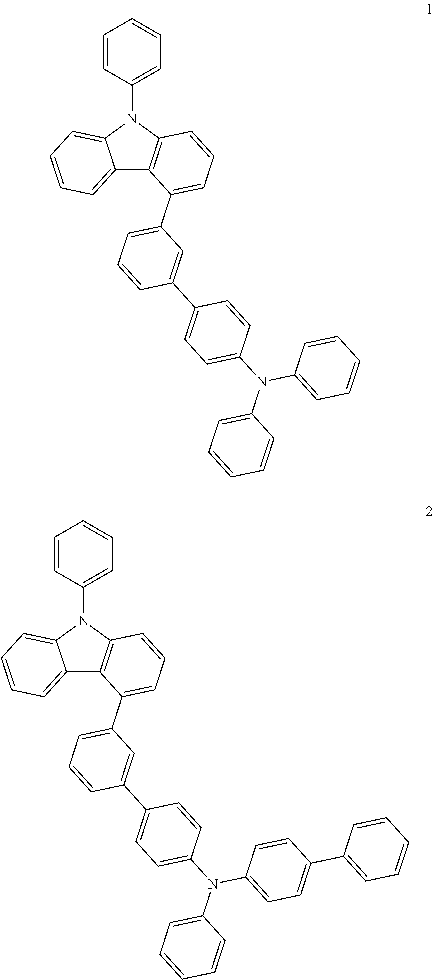 [SMILES: c1ccc(N(c2ccccc2)c2ccc(-c3cccc(-c4cccc5c4c4ccccc4n5-c4ccccc4)c3)cc2)cc1 ; c1ccc(-c2ccc(N(c3ccccc3)c3ccc(-c4cccc(-c5cccc6c5c5ccccc5n6-c5ccccc5)c4)cc3)cc2)cc1]

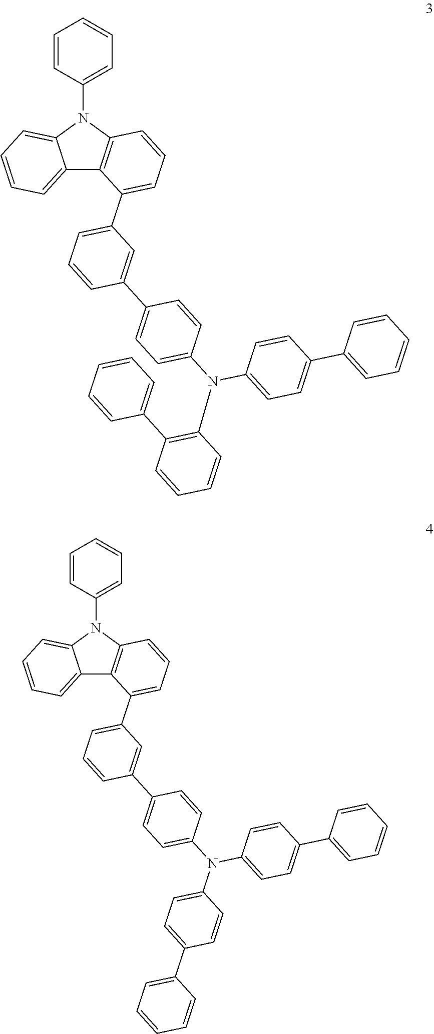 [SMILES: c1ccc(-c2ccc(N(c3ccc(-c4cccc(-c5cccc6c5c5ccccc5n6-c5ccccc5)c4)cc3)c3ccccc3-c3ccccc3)cc2)cc1 ; c1ccc(-c2ccc(N(c3ccc(-c4ccccc4)cc3)c3ccc(-c4cccc(-c5cccc6c5c5ccccc5n6-c5ccccc5)c4)cc3)cc2)cc1]

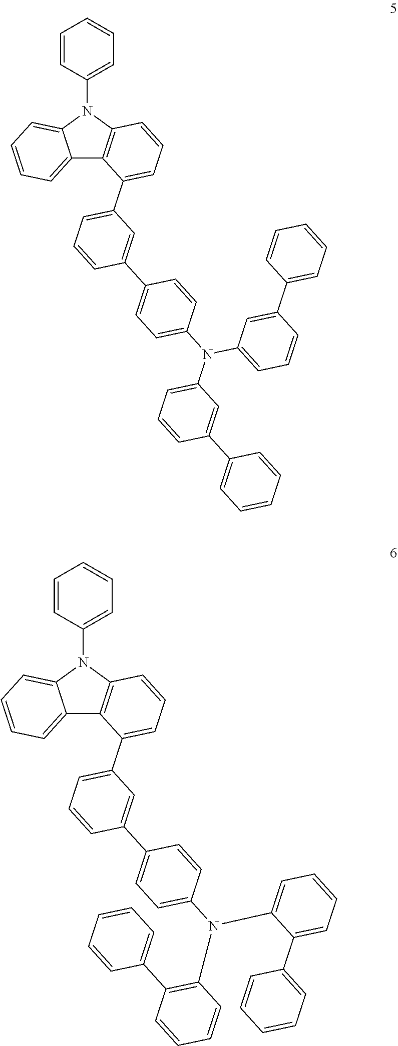 [SMILES: c1ccc(-c2cccc(N(c3ccc(-c4cccc(-c5cccc6c5c5ccccc5n6-c5ccccc5)c4)cc3)c3cccc(-c4ccccc4)c3)c2)cc1 ; c1ccc(-c2ccccc2N(c2ccc(-c3cccc(-c4cccc5c4c4ccccc4n5-c4ccccc4)c3)cc2)c2ccccc2-c2ccccc2)cc1]

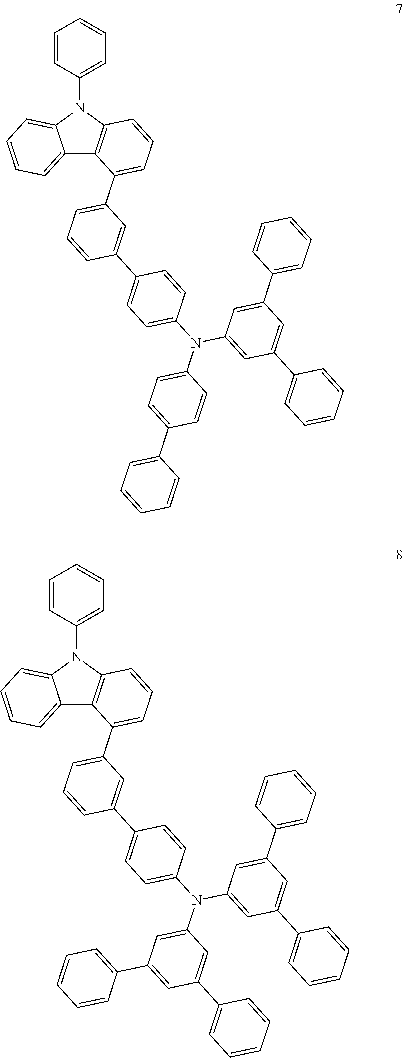 [SMILES: c1ccc(-c2ccc(N(c3ccc(-c4cccc(-c5cccc6c5c5ccccc5n6-c5ccccc5)c4)cc3)c3cc(-c4ccccc4)cc(-c4ccccc4)c3)cc2)cc1 ; c1ccc(-c2cc(-c3ccccc3)cc(N(c3ccc(-c4cccc(-c5cccc6c5c5ccccc5n6-c5ccccc5)c4)cc3)c3cc(-c4ccccc4)cc(-c4ccccc4)c3)c2)cc1]

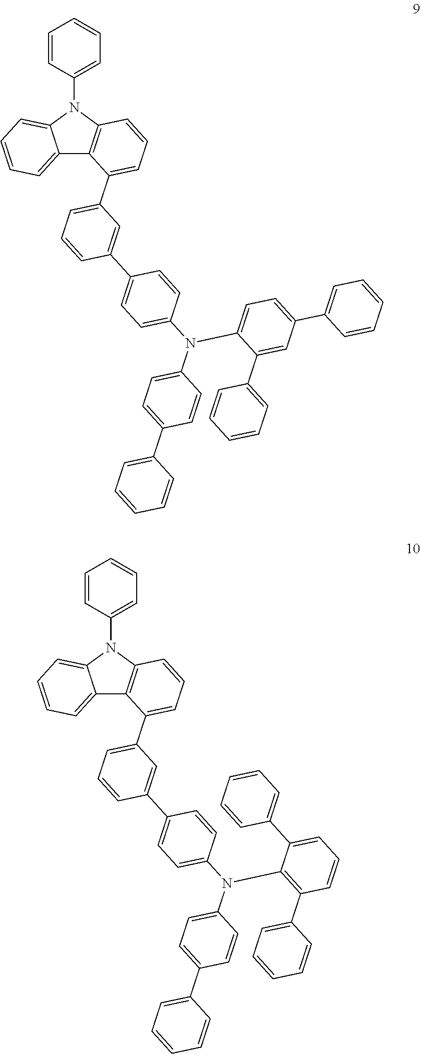 [SMILES: c1ccc(-c2ccc(N(c3ccc(-c4cccc(-c5cccc6c5c5ccccc5n6-c5ccccc5)c4)cc3)c3ccc(-c4ccccc4)cc3-c3ccccc3)cc2)cc1 ; c1ccc(-c2ccc(N(c3ccc(-c4cccc(-c5cccc6c5c5ccccc5n6-c5ccccc5)c4)cc3)c3c(-c4ccccc4)cccc3-c3ccccc3)cc2)cc1]

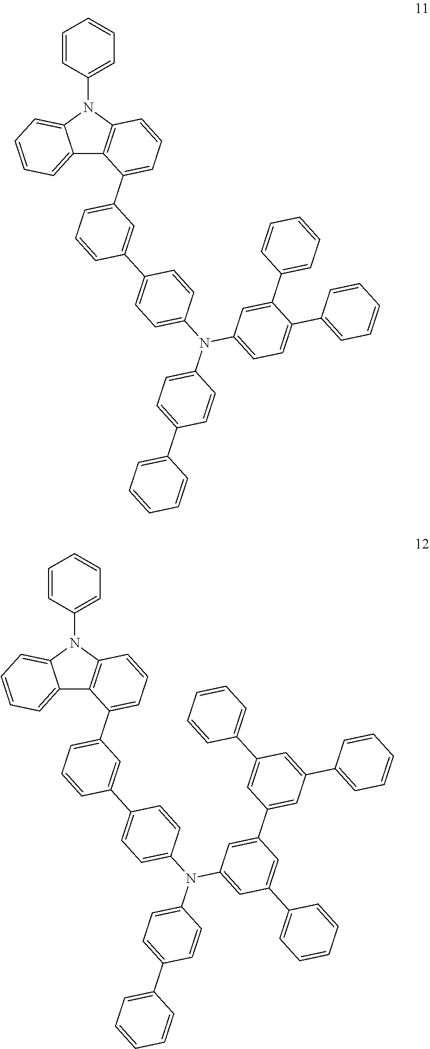 [SMILES: c1ccc(-c2ccc(N(c3ccc(-c4cccc(-c5cccc6c5c5ccccc5n6-c5ccccc5)c4)cc3)c3ccc(-c4ccccc4)c(-c4ccccc4)c3)cc2)cc1 ; c1ccc(-c2ccc(N(c3ccc(-c4cccc(-c5cccc6c5c5ccccc5n6-c5ccccc5)c4)cc3)c3cc(-c4ccccc4)cc(-c4cc(-c5ccccc5)cc(-c5ccccc5)c4)c3)cc2)cc1]

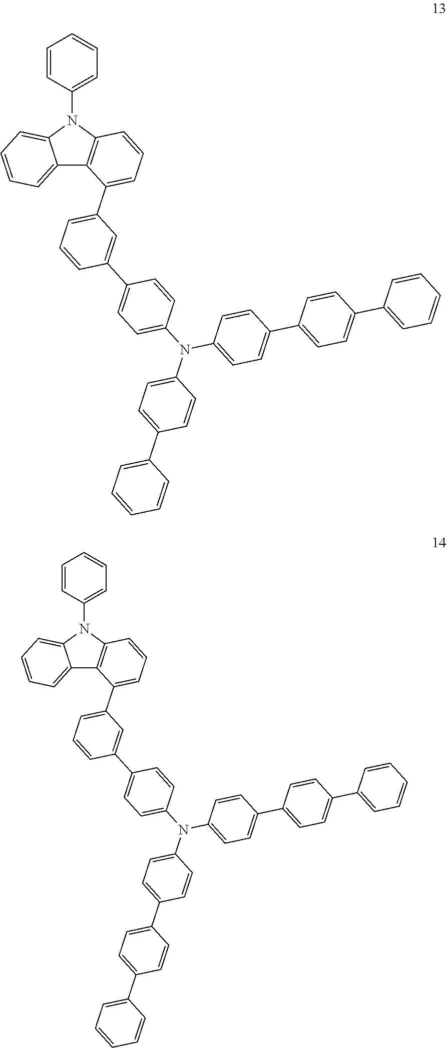 [SMILES: c1ccc(-c2ccc(-c3ccc(N(c4ccc(-c5ccccc5)cc4)c4ccc(-c5cccc(-c6cccc7c6c6ccccc6n7-c6ccccc6)c5)cc4)cc3)cc2)cc1 ; c1ccc(-c2ccc(-c3ccc(N(c4ccc(-c5ccc(-c6ccccc6)cc5)cc4)c4ccc(-c5cccc(-c6cccc7c6c6ccccc6n7-c6ccccc6)c5)cc4)cc3)cc2)cc1]

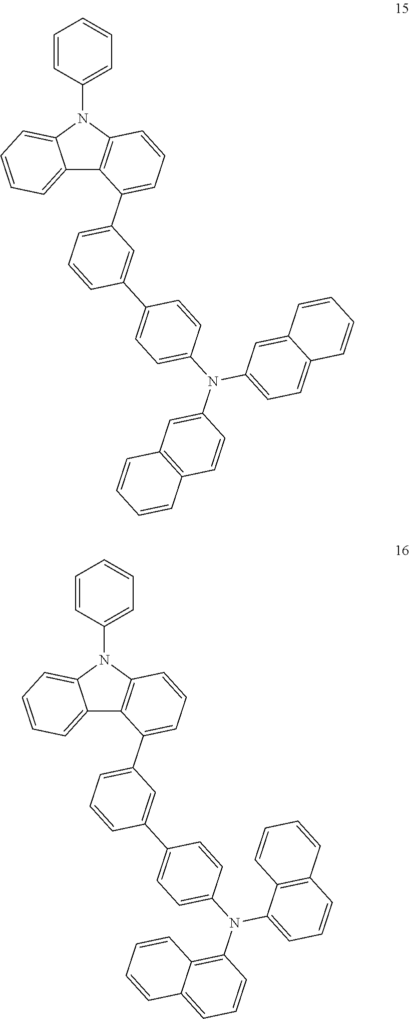 [SMILES: c1ccc(-n2c3ccccc3c3c(-c4cccc(-c5ccc(N(c6ccc7ccccc7c6)c6ccc7ccccc7c6)cc5)c4)cccc32)cc1 ; c1ccc(-n2c3ccccc3c3c(-c4cccc(-c5ccc(N(c6cccc7ccccc67)c6cccc7ccccc67)cc5)c4)cccc32)cc1]

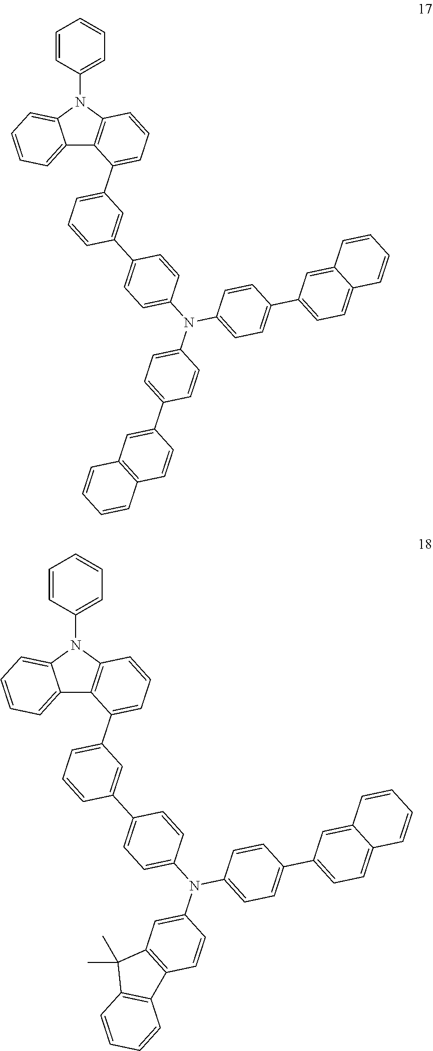 [SMILES: c1ccc(-n2c3ccccc3c3c(-c4cccc(-c5ccc(N(c6ccc(-c7ccc8ccccc8c7)cc6)c6ccc(-c7ccc8ccccc8c7)cc6)cc5)c4)cccc32)cc1 ; CC1(C)c2ccccc2-c2ccc(N(c3ccc(-c4cccc(-c5cccc6c5c5ccccc5n6-c5ccccc5)c4)cc3)c3ccc(-c4ccc5ccccc5c4)cc3)cc21]

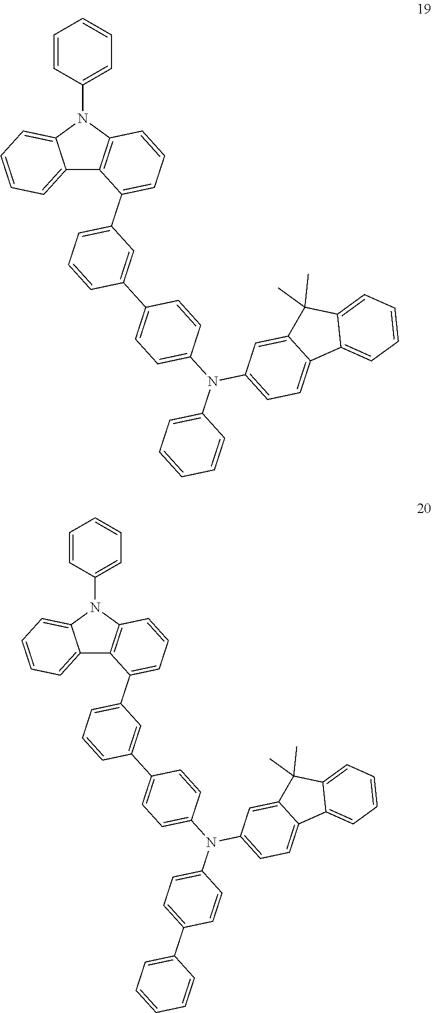 [SMILES: CC1(C)c2ccccc2-c2ccc(N(c3ccccc3)c3ccc(-c4cccc(-c5cccc6c5c5ccccc5n6-c5ccccc5)c4)cc3)cc21 ; CC1(C)c2ccccc2-c2ccc(N(c3ccc(-c4ccccc4)cc3)c3ccc(-c4cccc(-c5cccc6c5c5ccccc5n6-c5ccccc5)c4)cc3)cc21]

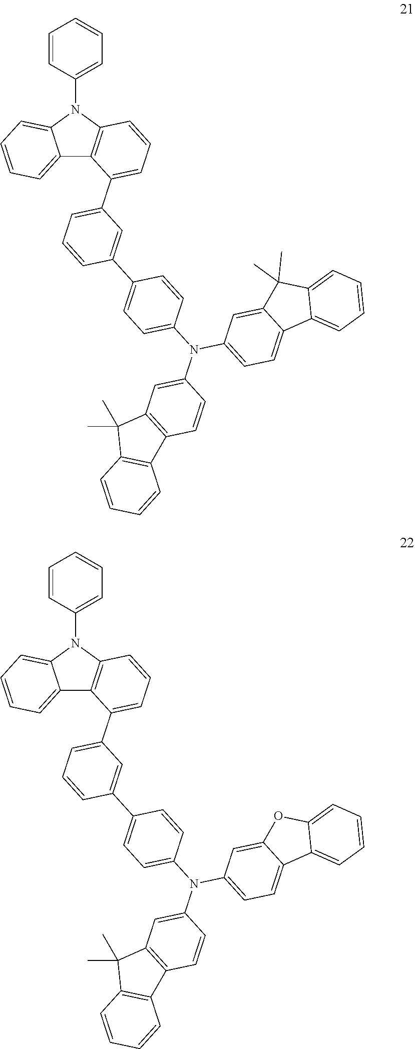 [SMILES: CC1(C)c2ccccc2-c2ccc(N(c3ccc(-c4cccc(-c5cccc6c5c5ccccc5n6-c5ccccc5)c4)cc3)c3ccc4c(c3)C(C)(C)c3ccccc3-4)cc21 ; CC1(C)c2ccccc2-c2ccc(N(c3ccc(-c4cccc(-c5cccc6c5c5ccccc5n6-c5ccccc5)c4)cc3)c3ccc4c(c3)oc3ccccc34)cc21]

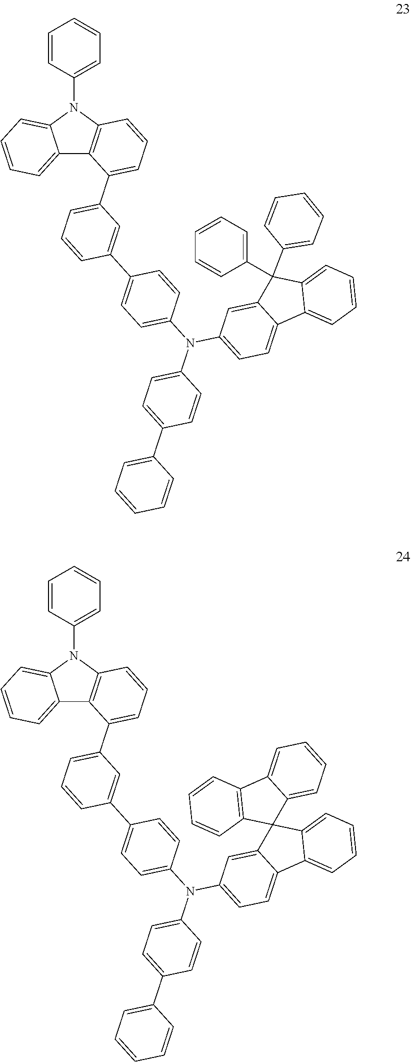 [SMILES: c1ccc(-c2ccc(N(c3ccc(-c4cccc(-c5cccc6c5c5ccccc5n6-c5ccccc5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5cccc6c5c5ccccc5n6-c5ccccc5)c4)cc3)c3ccc4c(c3)C3(c5ccccc5-c5ccccc53)c3ccccc3-4)cc2)cc1]

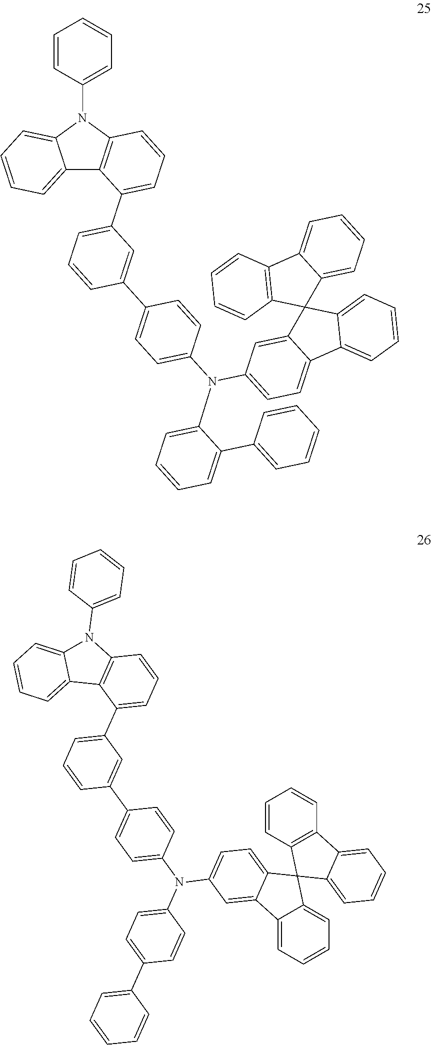 [SMILES: c1ccc(-c2ccccc2N(c2ccc(-c3cccc(-c4cccc5c4c4ccccc4n5-c4ccccc4)c3)cc2)c2ccc3c(c2)C2(c4ccccc4-c4ccccc42)c2ccccc2-3)cc1 ; c1ccc(-c2ccc(N(c3ccc(-c4cccc(-c5cccc6c5c5ccccc5n6-c5ccccc5)c4)cc3)c3ccc4c(c3)-c3ccccc3C43c4ccccc4-c4ccccc43)cc2)cc1]

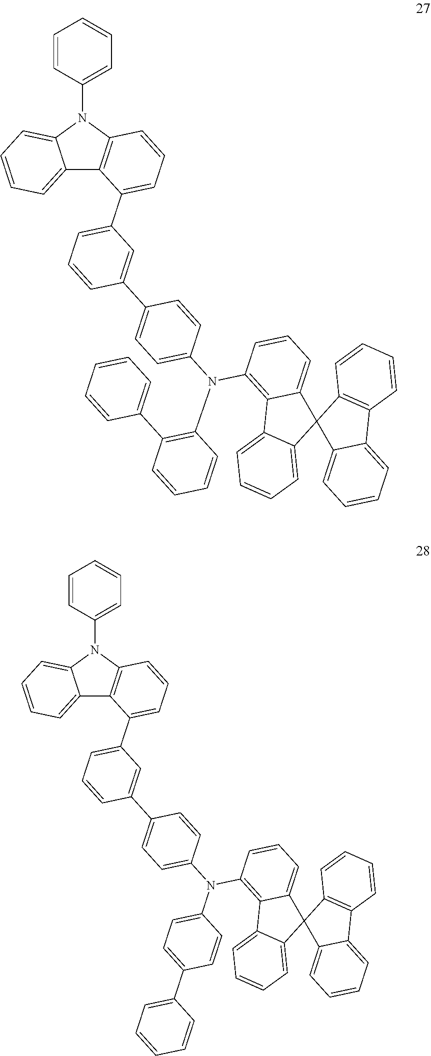 [SMILES: c1ccc(-c2ccccc2N(c2ccc(-c3cccc(-c4cccc5c4c4ccccc4n5-c4ccccc4)c3)cc2)c2cccc3c2-c2ccccc2C32c3ccccc3-c3ccccc32)cc1 ; c1ccc(-c2ccc(N(c3ccc(-c4cccc(-c5cccc6c5c5ccccc5n6-c5ccccc5)c4)cc3)c3cccc4c3-c3ccccc3C43c4ccccc4-c4ccccc43)cc2)cc1]

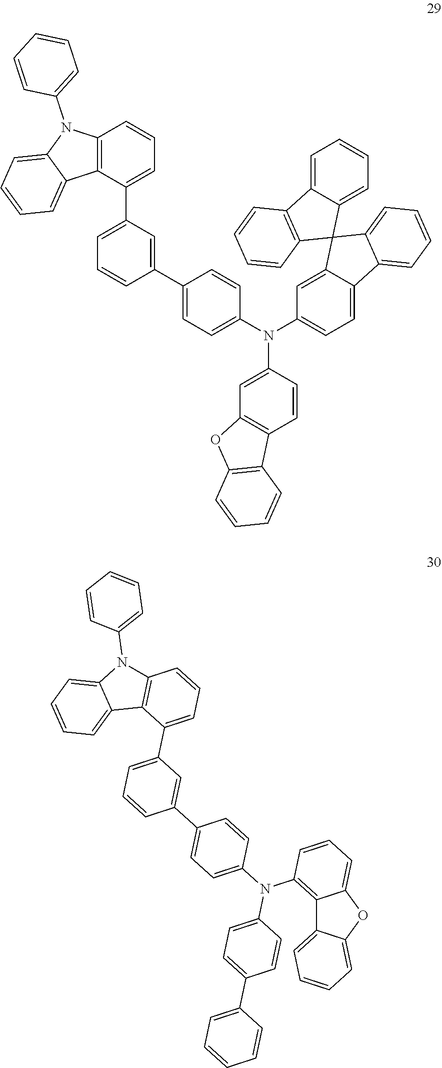 [SMILES: c1ccc(-n2c3ccccc3c3c(-c4cccc(-c5ccc(N(c6ccc7c(c6)C6(c8ccccc8-c8ccccc86)c6ccccc6-7)c6ccc7c(c6)oc6ccccc67)cc5)c4)cccc32)cc1 ; c1ccc(-c2ccc(N(c3ccc(-c4cccc(-c5cccc6c5c5ccccc5n6-c5ccccc5)c4)cc3)c3cccc4oc5ccccc5c34)cc2)cc1]

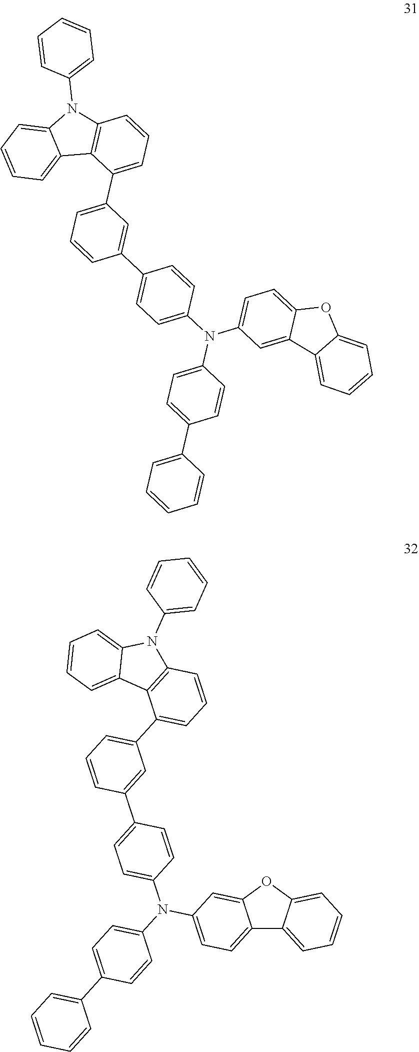 [SMILES: c1ccc(-c2ccc(N(c3ccc(-c4cccc(-c5cccc6c5c5ccccc5n6-c5ccccc5)c4)cc3)c3ccc4oc5ccccc5c4c3)cc2)cc1 ; c1ccc(-c2ccc(N(c3ccc(-c4cccc(-c5cccc6c5c5ccccc5n6-c5ccccc5)c4)cc3)c3ccc4c(c3)oc3ccccc34)cc2)cc1]

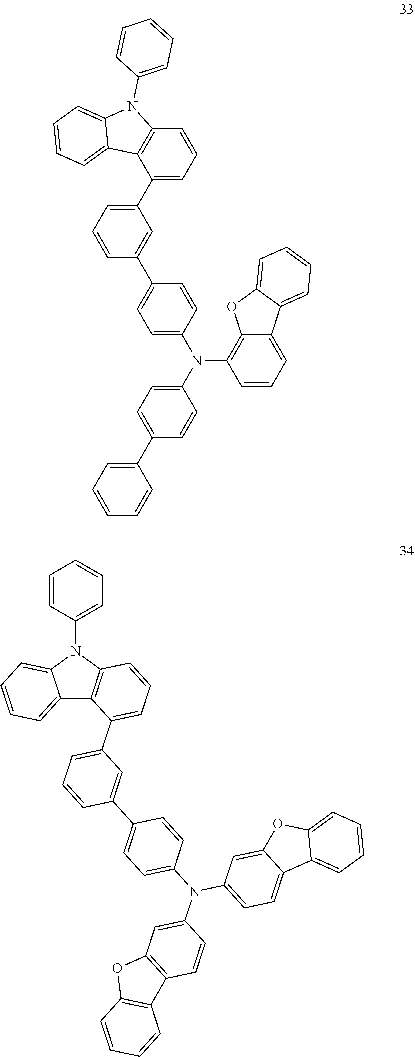 [SMILES: c1ccc(-c2ccc(N(c3ccc(-c4cccc(-c5cccc6c5c5ccccc5n6-c5ccccc5)c4)cc3)c3cccc4c3oc3ccccc34)cc2)cc1 ; c1ccc(-n2c3ccccc3c3c(-c4cccc(-c5ccc(N(c6ccc7c(c6)oc6ccccc67)c6ccc7c(c6)oc6ccccc67)cc5)c4)cccc32)cc1]

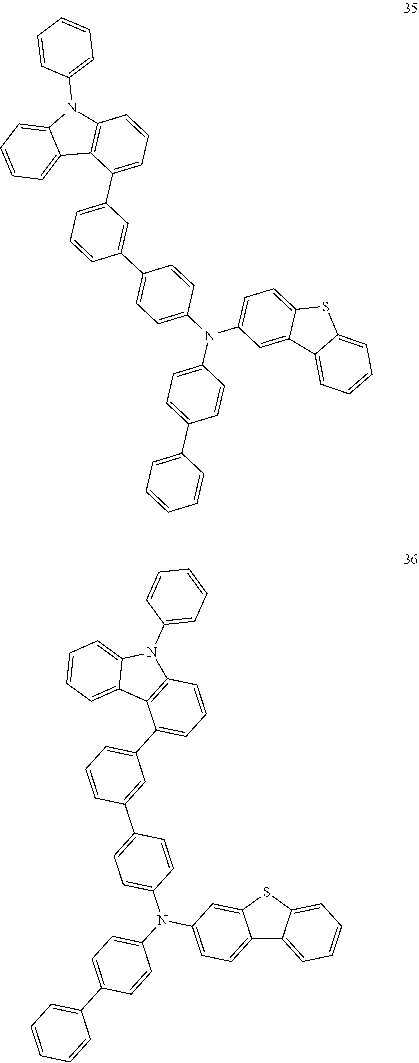 [SMILES: c1ccc(-c2ccc(N(c3ccc(-c4cccc(-c5cccc6c5c5ccccc5n6-c5ccccc5)c4)cc3)c3ccc4sc5ccccc5c4c3)cc2)cc1 ; c1ccc(-c2ccc(N(c3ccc(-c4cccc(-c5cccc6c5c5ccccc5n6-c5ccccc5)c4)cc3)c3ccc4c(c3)sc3ccccc34)cc2)cc1]

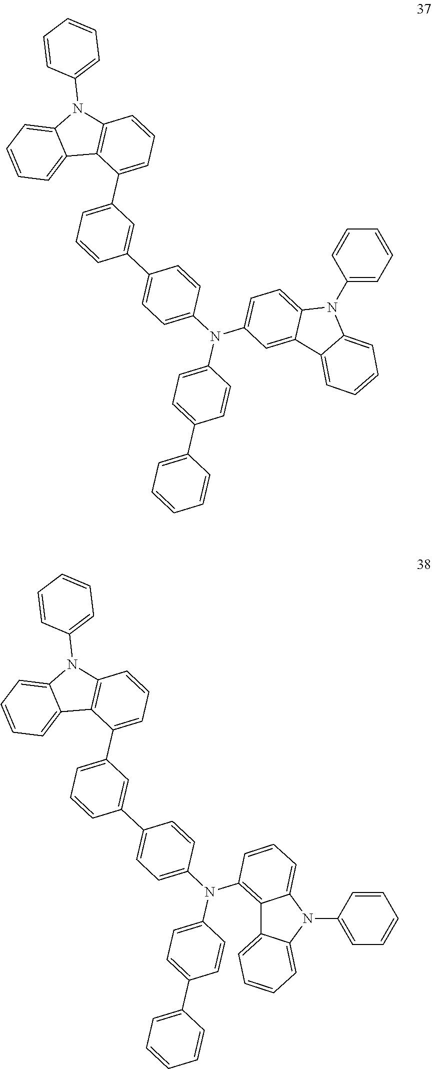 [SMILES: c1ccc(-c2ccc(N(c3ccc(-c4cccc(-c5cccc6c5c5ccccc5n6-c5ccccc5)c4)cc3)c3ccc4c(c3)c3ccccc3n4-c3ccccc3)cc2)cc1 ; c1ccc(-c2ccc(N(c3ccc(-c4cccc(-c5cccc6c5c5ccccc5n6-c5ccccc5)c4)cc3)c3cccc4c3c3ccccc3n4-c3ccccc3)cc2)cc1]

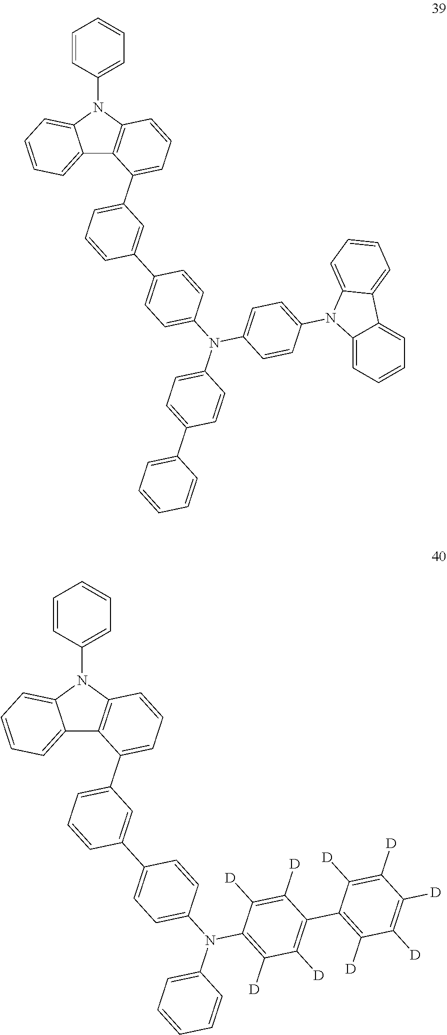 [SMILES: c1ccc(-c2ccc(N(c3ccc(-c4cccc(-c5cccc6c5c5ccccc5n6-c5ccccc5)c4)cc3)c3ccc(-n4c5ccccc5c5ccccc54)cc3)cc2)cc1 ; [2H]c1c([2H])c([2H])c(-c2c([2H])c([2H])c(N(c3ccccc3)c3ccc(-c4cccc(-c5cccc6c5c5ccccc5n6-c5ccccc5)c4)cc3)c([2H])c2[2H])c([2H])c1[2H]]

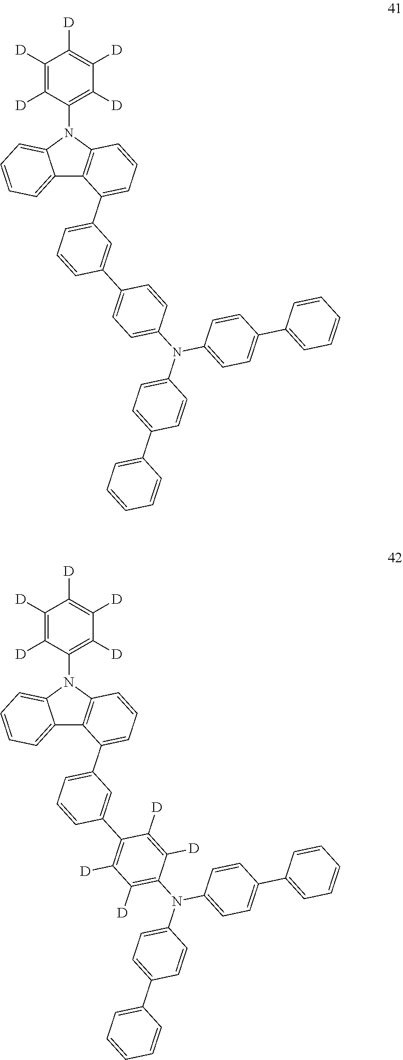 [SMILES: [2H]c1c([2H])c([2H])c(-n2c3ccccc3c3c(-c4cccc(-c5ccc(N(c6ccc(-c7ccccc7)cc6)c6ccc(-c7ccccc7)cc6)cc5)c4)cccc32)c([2H])c1[2H] ; [2H]c1c([2H])c([2H])c(-n2c3ccccc3c3c(-c4cccc(-c5c([2H])c([2H])c(N(c6ccc(-c7ccccc7)cc6)c6ccc(-c7ccccc7)cc6)c([2H])c5[2H])c4)cccc32)c([2H])c1[2H]]

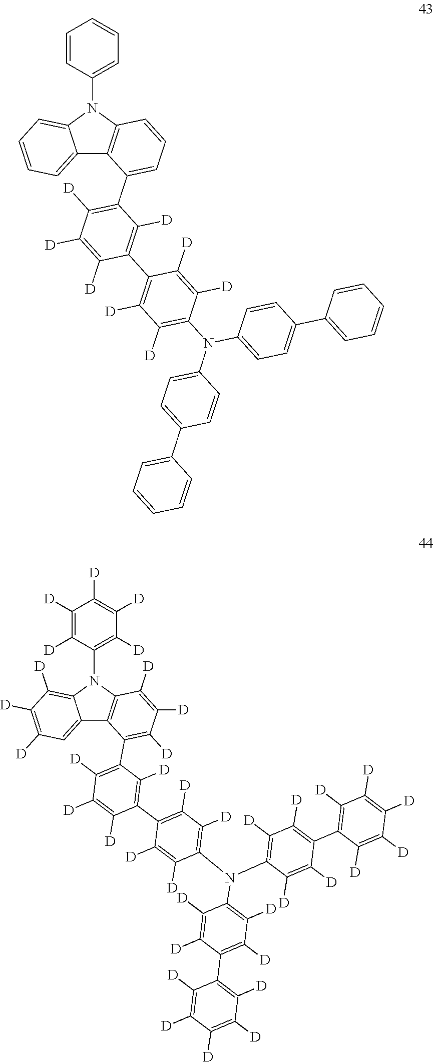 [SMILES: [2H]c1c([2H])c(-c2c([2H])c([2H])c(N(c3ccc(-c4ccccc4)cc3)c3ccc(-c4ccccc4)cc3)c([2H])c2[2H])c([2H])c(-c2cccc3c2c2ccccc2n3-c2ccccc2)c1[2H] ; [2H]c1cc2c3c(-c4c([2H])c([2H])c([2H])c(-c5c([2H])c([2H])c(N(c6c([2H])c([2H])c(-c7c([2H])c([2H])c([2H])c([2H])c7[2H])c([2H])c6[2H])c6c([2H])c([2H])c(-c7c([2H])c([2H])c([2H])c([2H])c7[2H])c([2H])c6[2H])c([2H])c5[2H])c4[2H])c([2H])c([2H])c([2H])c3n(-c3c([2H])c([2H])c([2H])c([2H])c3[2H])c2c([2H])c1[2H]]

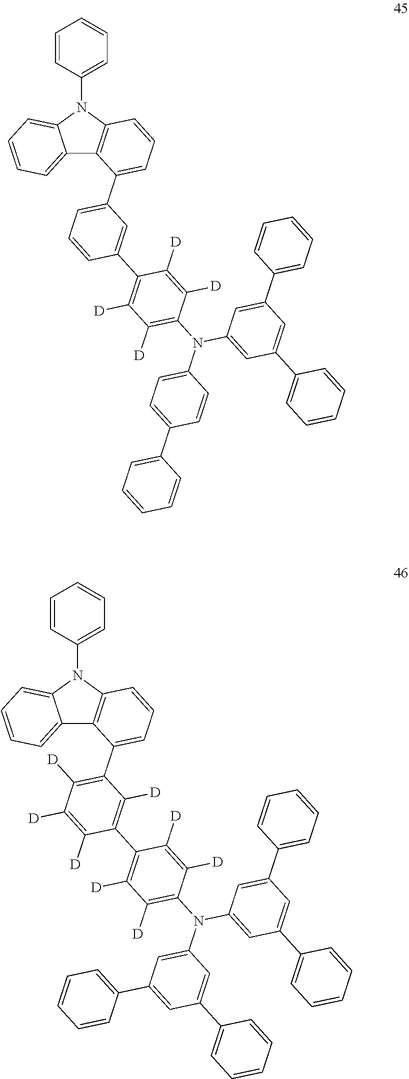 [SMILES: [2H]c1c([2H])c(N(c2ccc(-c3ccccc3)cc2)c2cc(-c3ccccc3)cc(-c3ccccc3)c2)c([2H])c([2H])c1-c1cccc(-c2cccc3c2c2ccccc2n3-c2ccccc2)c1 ; [2H]c1c([2H])c(-c2c([2H])c([2H])c(N(c3cc(-c4ccccc4)cc(-c4ccccc4)c3)c3cc(-c4ccccc4)cc(-c4ccccc4)c3)c([2H])c2[2H])c([2H])c(-c2cccc3c2c2ccccc2n3-c2ccccc2)c1[2H]]

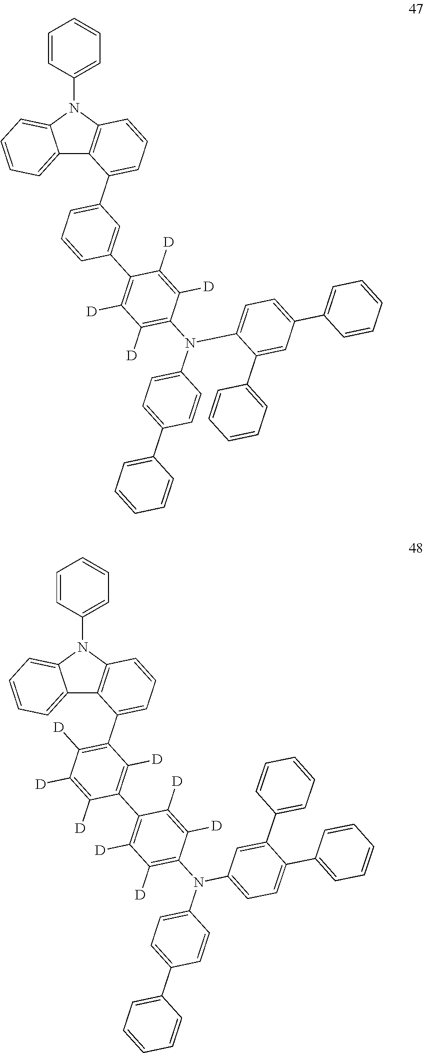 [SMILES: [2H]c1c([2H])c(N(c2ccc(-c3ccccc3)cc2)c2ccc(-c3ccccc3)cc2-c2ccccc2)c([2H])c([2H])c1-c1cccc(-c2cccc3c2c2ccccc2n3-c2ccccc2)c1 ; [2H]c1c([2H])c(-c2c([2H])c([2H])c(N(c3ccc(-c4ccccc4)cc3)c3ccc(-c4ccccc4)c(-c4ccccc4)c3)c([2H])c2[2H])c([2H])c(-c2cccc3c2c2ccccc2n3-c2ccccc2)c1[2H]]

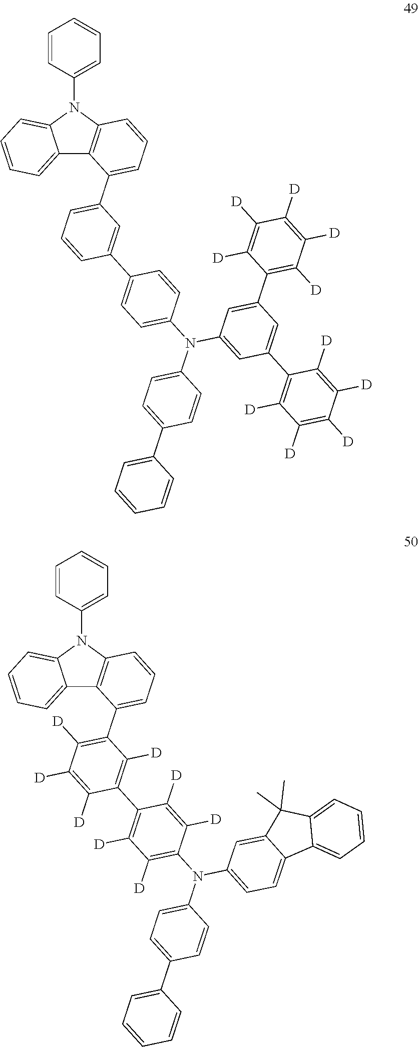 [SMILES: [2H]c1c([2H])c([2H])c(-c2cc(-c3c([2H])c([2H])c([2H])c([2H])c3[2H])cc(N(c3ccc(-c4ccccc4)cc3)c3ccc(-c4cccc(-c5cccc6c5c5ccccc5n6-c5ccccc5)c4)cc3)c2)c([2H])c1[2H] ; [2H]c1c([2H])c(-c2c([2H])c([2H])c(N(c3ccc(-c4ccccc4)cc3)c3ccc4c(c3)C(C)(C)c3ccccc3-4)c([2H])c2[2H])c([2H])c(-c2cccc3c2c2ccccc2n3-c2ccccc2)c1[2H]]

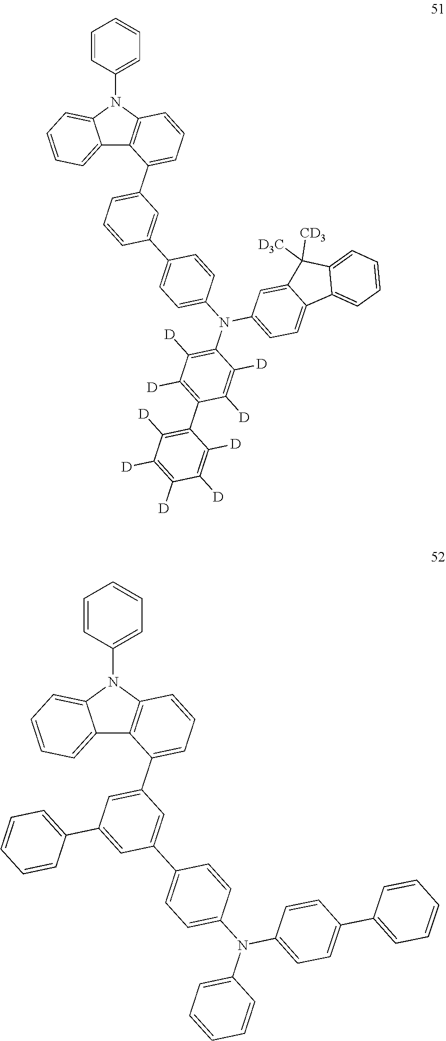 [SMILES: [2H]c1c([2H])c([2H])c(-c2c([2H])c([2H])c(N(c3ccc(-c4cccc(-c5cccc6c5c5ccccc5n6-c5ccccc5)c4)cc3)c3ccc4c(c3)C(C([2H])([2H])[2H])(C([2H])([2H])[2H])c3ccccc3-4)c([2H])c2[2H])c([2H])c1[2H] ; c1ccc(-c2ccc(N(c3ccccc3)c3ccc(-c4cc(-c5ccccc5)cc(-c5cccc6c5c5ccccc5n6-c5ccccc5)c4)cc3)cc2)cc1]

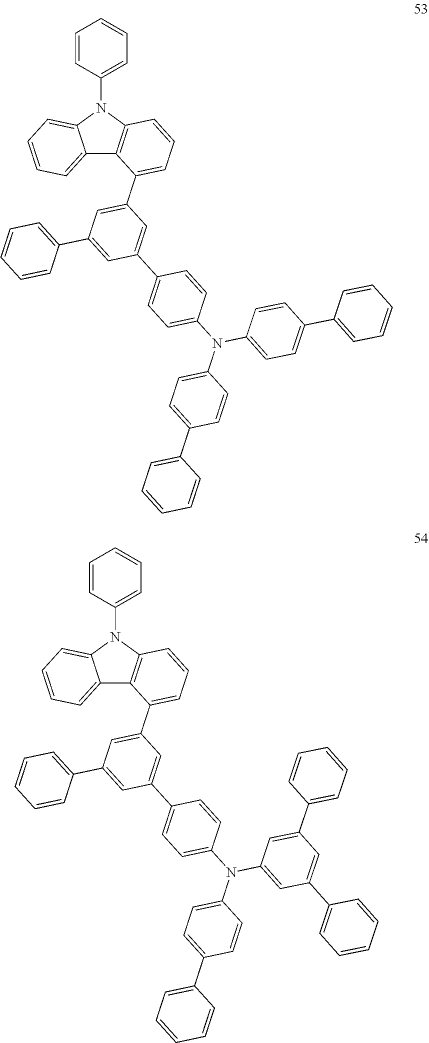 [SMILES: c1ccc(-c2ccc(N(c3ccc(-c4ccccc4)cc3)c3ccc(-c4cc(-c5ccccc5)cc(-c5cccc6c5c5ccccc5n6-c5ccccc5)c4)cc3)cc2)cc1 ; c1ccc(-c2ccc(N(c3ccc(-c4cc(-c5ccccc5)cc(-c5cccc6c5c5ccccc5n6-c5ccccc5)c4)cc3)c3cc(-c4ccccc4)cc(-c4ccccc4)c3)cc2)cc1]

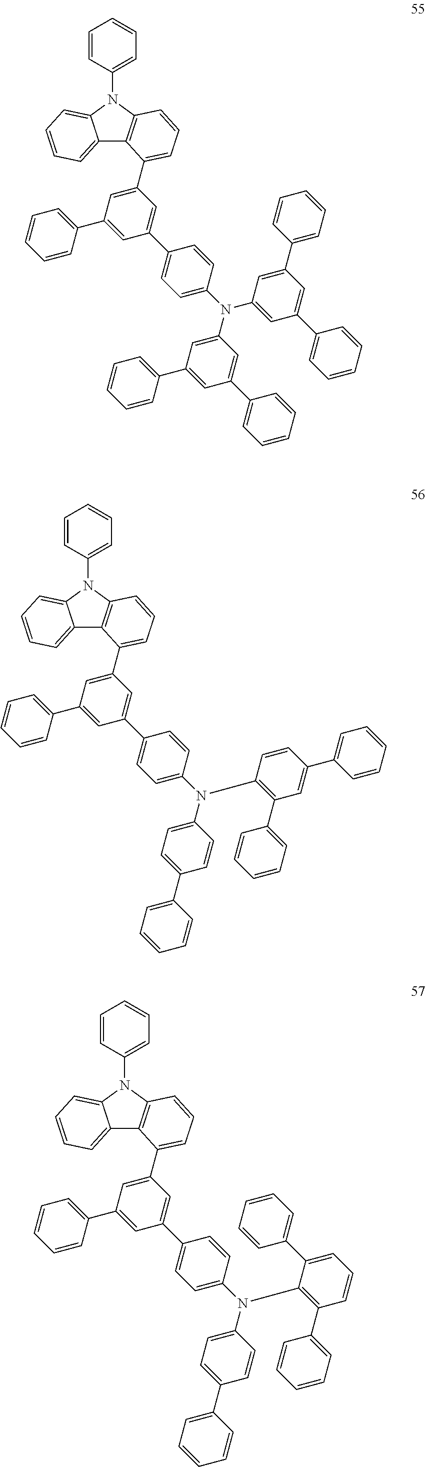 [SMILES: c1ccc(-c2cc(-c3ccc(N(c4cc(-c5ccccc5)cc(-c5ccccc5)c4)c4cc(-c5ccccc5)cc(-c5ccccc5)c4)cc3)cc(-c3cccc4c3c3ccccc3n4-c3ccccc3)c2)cc1 ; c1ccc(-c2ccc(N(c3ccc(-c4cc(-c5ccccc5)cc(-c5cccc6c5c5ccccc5n6-c5ccccc5)c4)cc3)c3ccc(-c4ccccc4)cc3-c3ccccc3)cc2)cc1 ; c1ccc(-c2ccc(N(c3ccc(-c4cc(-c5ccccc5)cc(-c5cccc6c5c5ccccc5n6-c5ccccc5)c4)cc3)c3c(-c4ccccc4)cccc3-c3ccccc3)cc2)cc1]

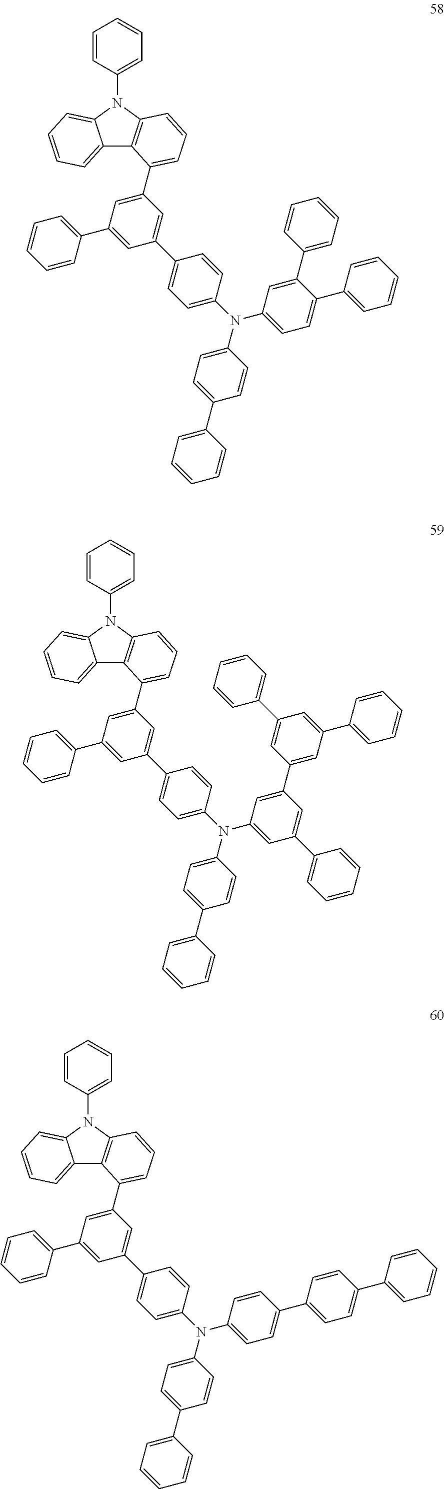 [SMILES: c1ccc(-c2ccc(N(c3ccc(-c4cc(-c5ccccc5)cc(-c5cccc6c5c5ccccc5n6-c5ccccc5)c4)cc3)c3ccc(-c4ccccc4)c(-c4ccccc4)c3)cc2)cc1 ; c1ccc(-c2ccc(N(c3ccc(-c4cc(-c5ccccc5)cc(-c5cccc6c5c5ccccc5n6-c5ccccc5)c4)cc3)c3cc(-c4ccccc4)cc(-c4cc(-c5ccccc5)cc(-c5ccccc5)c4)c3)cc2)cc1 ; c1ccc(-c2ccc(-c3ccc(N(c4ccc(-c5ccccc5)cc4)c4ccc(-c5cc(-c6ccccc6)cc(-c6cccc7c6c6ccccc6n7-c6ccccc6)c5)cc4)cc3)cc2)cc1]

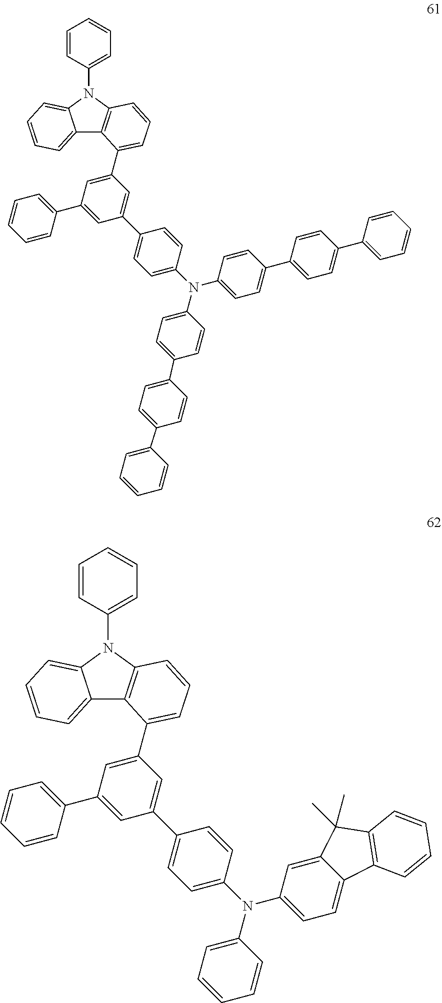 [SMILES: c1ccc(-c2ccc(-c3ccc(N(c4ccc(-c5ccc(-c6ccccc6)cc5)cc4)c4ccc(-c5cc(-c6ccccc6)cc(-c6cccc7c6c6ccccc6n7-c6ccccc6)c5)cc4)cc3)cc2)cc1 ; CC1(C)c2ccccc2-c2ccc(N(c3ccccc3)c3ccc(-c4cc(-c5ccccc5)cc(-c5cccc6c5c5ccccc5n6-c5ccccc5)c4)cc3)cc21]

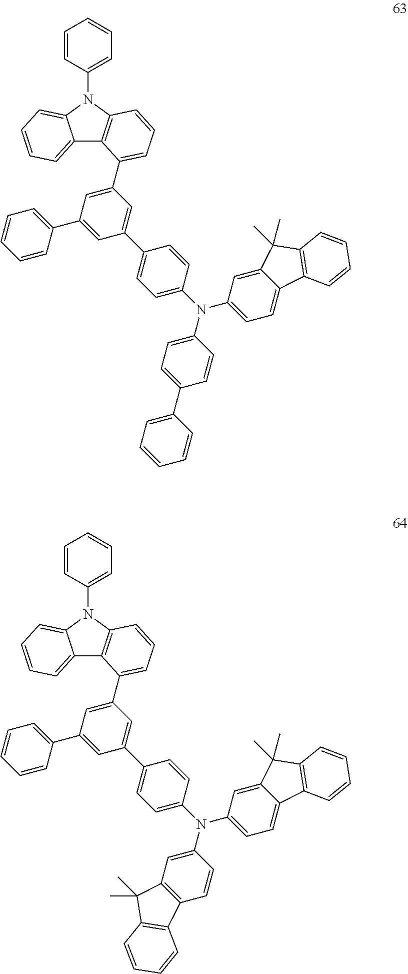 [SMILES: CC1(C)c2ccccc2-c2ccc(N(c3ccc(-c4ccccc4)cc3)c3ccc(-c4cc(-c5ccccc5)cc(-c5cccc6c5c5ccccc5n6-c5ccccc5)c4)cc3)cc21 ; CC1(C)c2ccccc2-c2ccc(N(c3ccc(-c4cc(-c5ccccc5)cc(-c5cccc6c5c5ccccc5n6-c5ccccc5)c4)cc3)c3ccc4c(c3)C(C)(C)c3ccccc3-4)cc21]

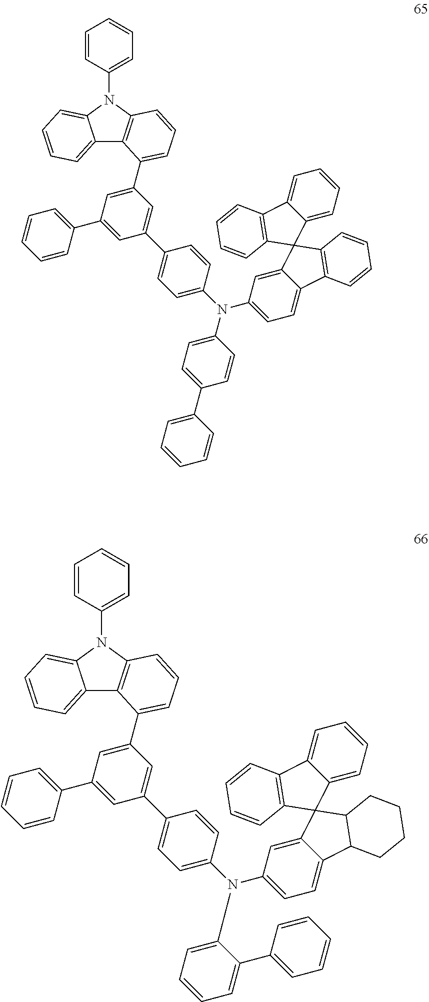 [SMILES: c1ccc(-c2ccc(N(c3ccc(-c4cc(-c5ccccc5)cc(-c5cccc6c5c5ccccc5n6-c5ccccc5)c4)cc3)c3ccc4c(c3)C3(c5ccccc5-c5ccccc53)c3ccccc3-4)cc2)cc1 ; c1ccc(-c2cc(-c3ccc(N(c4ccc5c(c4)C4(c6ccccc6-c6ccccc64)C4CCCCC54)c4ccccc4-c4ccccc4)cc3)cc(-c3cccc4c3c3ccccc3n4-c3ccccc3)c2)cc1]

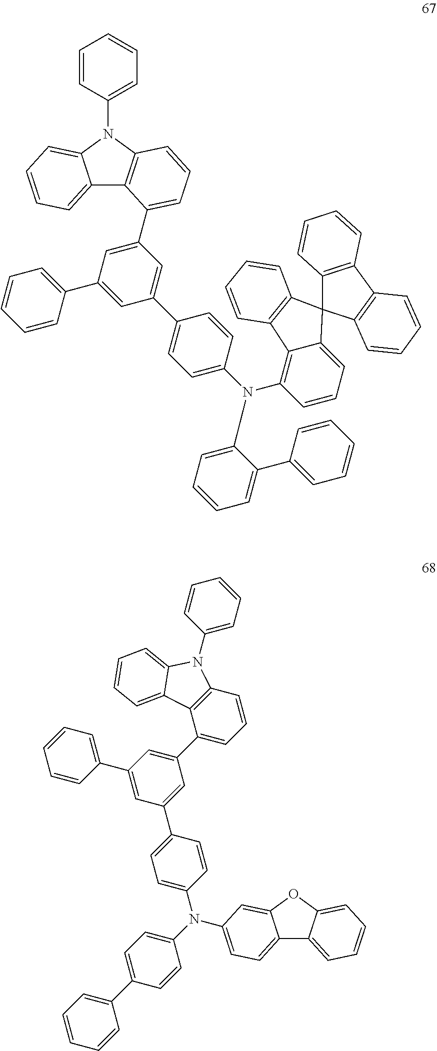 [SMILES: c1ccc(-c2cc(-c3ccc(N(c4ccccc4-c4ccccc4)c4cccc5c4-c4ccccc4C54c5ccccc5-c5ccccc54)cc3)cc(-c3cccc4c3c3ccccc3n4-c3ccccc3)c2)cc1 ; c1ccc(-c2ccc(N(c3ccc(-c4cc(-c5ccccc5)cc(-c5cccc6c5c5ccccc5n6-c5ccccc5)c4)cc3)c3ccc4c(c3)oc3ccccc34)cc2)cc1]

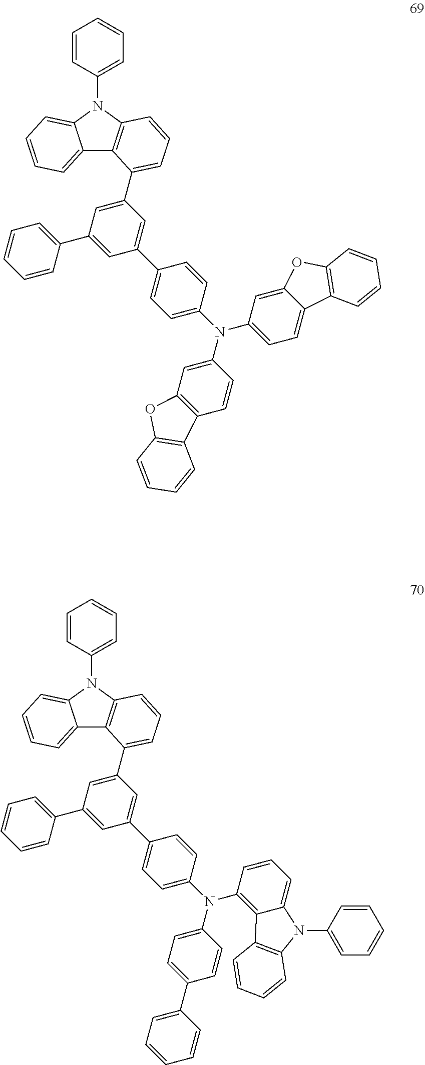 [SMILES: c1ccc(-c2cc(-c3ccc(N(c4ccc5c(c4)oc4ccccc45)c4ccc5c(c4)oc4ccccc45)cc3)cc(-c3cccc4c3c3ccccc3n4-c3ccccc3)c2)cc1 ; c1ccc(-c2ccc(N(c3ccc(-c4cc(-c5ccccc5)cc(-c5cccc6c5c5ccccc5n6-c5ccccc5)c4)cc3)c3cccc4c3c3ccccc3n4-c3ccccc3)cc2)cc1]

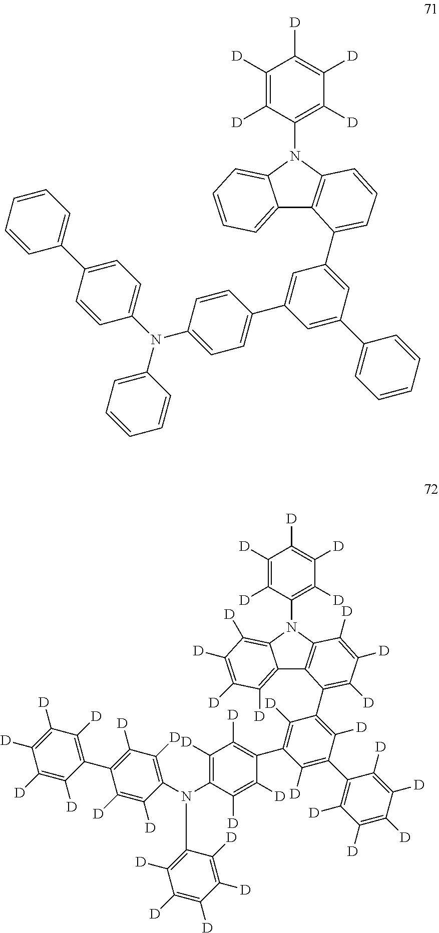 [SMILES: [2H]c1c([2H])c([2H])c(-n2c3ccccc3c3c(-c4cc(-c5ccccc5)cc(-c5ccc(N(c6ccccc6)c6ccc(-c7ccccc7)cc6)cc5)c4)cccc32)c([2H])c1[2H] ; [2H]c1c([2H])c([2H])c(-c2c([2H])c([2H])c(N(c3c([2H])c([2H])c([2H])c([2H])c3[2H])c3c([2H])c([2H])c(-c4c([2H])c(-c5c([2H])c([2H])c([2H])c([2H])c5[2H])c([2H])c(-c5c([2H])c([2H])c([2H])c6c5c5c([2H])c([2H])c([2H])c([2H])c5n6-c5c([2H])c([2H])c([2H])c([2H])c5[2H])c4[2H])c([2H])c3[2H])c([2H])c2[2H])c([2H])c1[2H]]

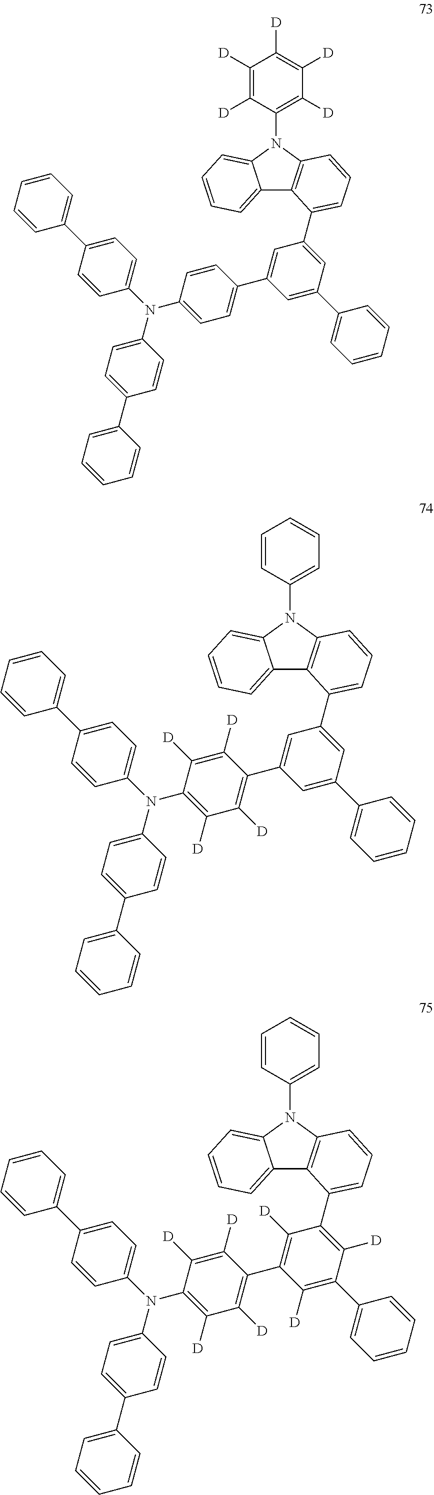 [SMILES: [2H]c1c([2H])c([2H])c(-n2c3ccccc3c3c(-c4cc(-c5ccccc5)cc(-c5ccc(N(c6ccc(-c7ccccc7)cc6)c6ccc(-c7ccccc7)cc6)cc5)c4)cccc32)c([2H])c1[2H] ; [2H]c1c([2H])c(N(c2ccc(-c3ccccc3)cc2)c2ccc(-c3ccccc3)cc2)c([2H])c([2H])c1-c1cc(-c2ccccc2)cc(-c2cccc3c2c2ccccc2n3-c2ccccc2)c1 ; [2H]c1c([2H])c(N(c2ccc(-c3ccccc3)cc2)c2ccc(-c3ccccc3)cc2)c([2H])c([2H])c1-c1c([2H])c(-c2ccccc2)c([2H])c(-c2cccc3c2c2ccccc2n3-c2ccccc2)c1[2H]]

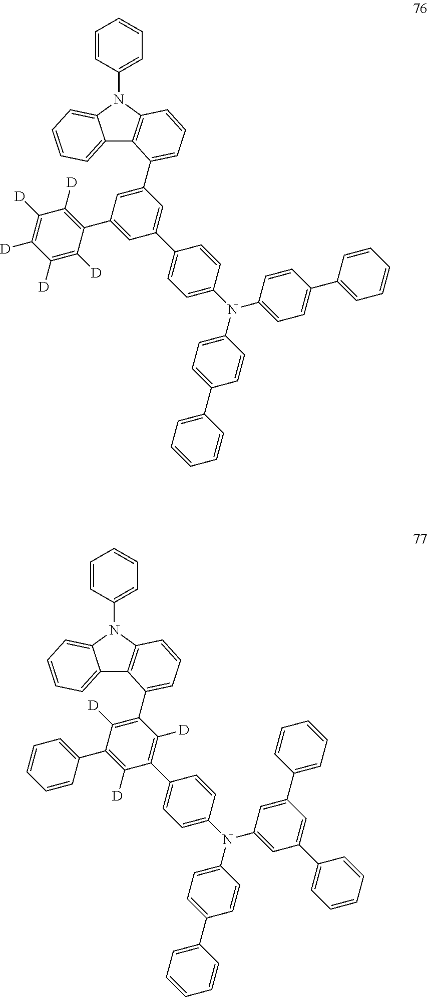 [SMILES: [2H]c1c([2H])c([2H])c(-c2cc(-c3ccc(N(c4ccc(-c5ccccc5)cc4)c4ccc(-c5ccccc5)cc4)cc3)cc(-c3cccc4c3c3ccccc3n4-c3ccccc3)c2)c([2H])c1[2H] ; [2H]c1c(-c2ccccc2)c([2H])c(-c2cccc3c2c2ccccc2n3-c2ccccc2)c([2H])c1-c1ccc(N(c2ccc(-c3ccccc3)cc2)c2cc(-c3ccccc3)cc(-c3ccccc3)c2)cc1]

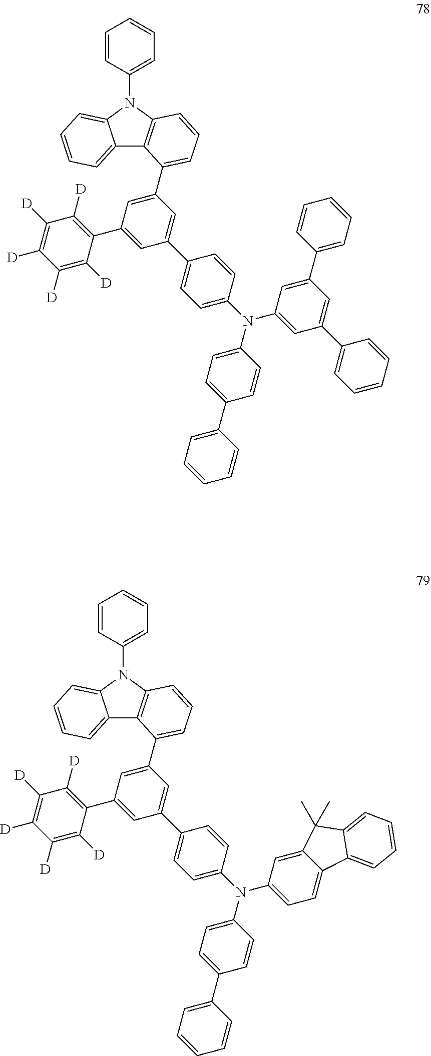 [SMILES: [2H]c1c([2H])c([2H])c(-c2cc(-c3ccc(N(c4ccc(-c5ccccc5)cc4)c4cc(-c5ccccc5)cc(-c5ccccc5)c4)cc3)cc(-c3cccc4c3c3ccccc3n4-c3ccccc3)c2)c([2H])c1[2H] ; [2H]c1c([2H])c([2H])c(-c2cc(-c3ccc(N(c4ccc(-c5ccccc5)cc4)c4ccc5c(c4)C(C)(C)c4ccccc4-5)cc3)cc(-c3cccc4c3c3ccccc3n4-c3ccccc3)c2)c([2H])c1[2H]]

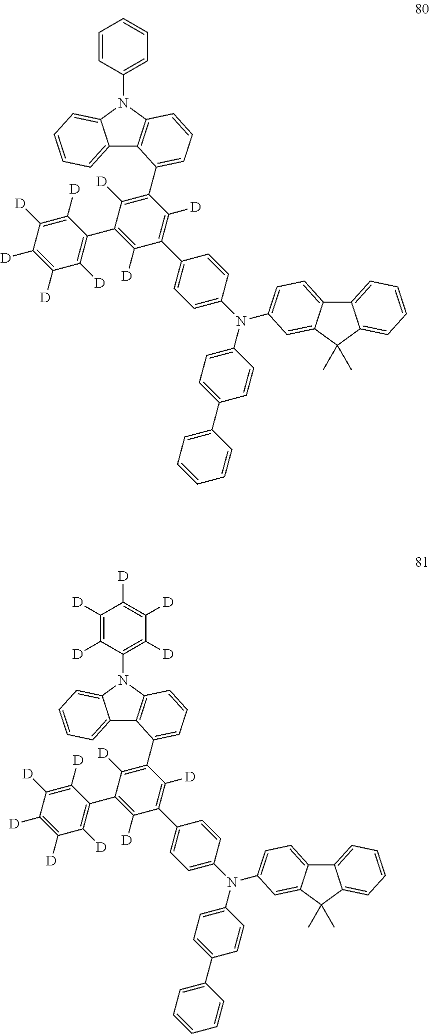 [SMILES: [2H]c1c([2H])c([2H])c(-c2c([2H])c(-c3ccc(N(c4ccc(-c5ccccc5)cc4)c4ccc5c(c4)C(C)(C)c4ccccc4-5)cc3)c([2H])c(-c3cccc4c3c3ccccc3n4-c3ccccc3)c2[2H])c([2H])c1[2H] ; [2H]c1c([2H])c([2H])c(-c2c([2H])c(-c3ccc(N(c4ccc(-c5ccccc5)cc4)c4ccc5c(c4)C(C)(C)c4ccccc4-5)cc3)c([2H])c(-c3cccc4c3c3ccccc3n4-c3c([2H])c([2H])c([2H])c([2H])c3[2H])c2[2H])c([2H])c1[2H]]

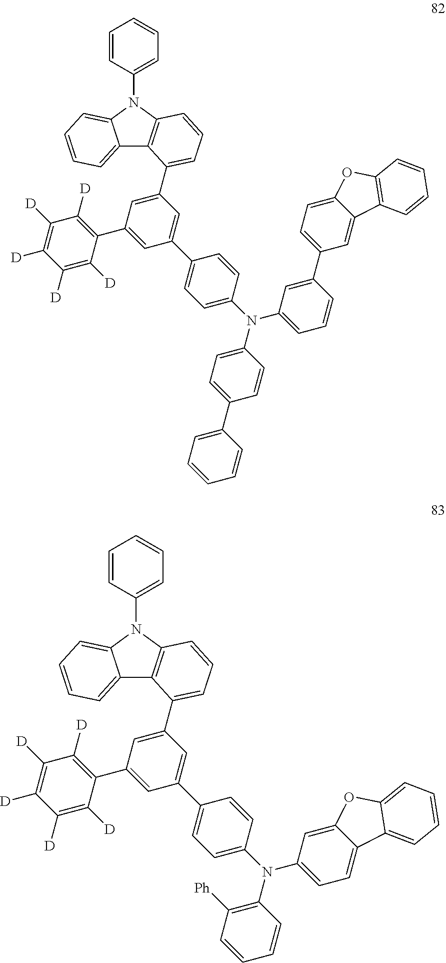 [SMILES: [2H]c1c([2H])c([2H])c(-c2cc(-c3ccc(N(c4ccc(-c5ccccc5)cc4)c4cccc(-c5ccc6oc7ccccc7c6c5)c4)cc3)cc(-c3cccc4c3c3ccccc3n4-c3ccccc3)c2)c([2H])c1[2H] ; [2H]c1c([2H])c([2H])c(-c2cc(-c3ccc(N(c4ccc5c(c4)oc4ccccc45)c4ccccc4-c4ccccc4)cc3)cc(-c3cccc4c3c3ccccc3n4-c3ccccc3)c2)c([2H])c1[2H]]

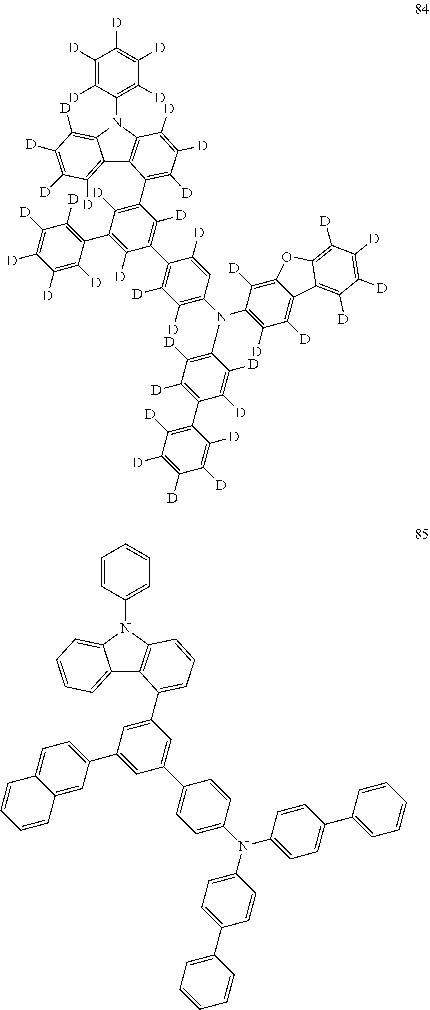 [SMILES: [2H]c1cc(N(c2c([2H])c([2H])c(-c3c([2H])c([2H])c([2H])c([2H])c3[2H])c([2H])c2[2H])c2c([2H])c([2H])c3c(oc4c([2H])c([2H])c([2H])c([2H])c43)c2[2H])c([2H])c([2H])c1-c1c([2H])c(-c2c([2H])c([2H])c([2H])c([2H])c2[2H])c([2H])c(-c2c([2H])c([2H])c([2H])c3c2c2c([2H])c([2H])c([2H])c([2H])c2n3-c2c([2H])c([2H])c([2H])c([2H])c2[2H])c1[2H] ; c1ccc(-c2ccc(N(c3ccc(-c4ccccc4)cc3)c3ccc(-c4cc(-c5ccc6ccccc6c5)cc(-c5cccc6c5c5ccccc5n6-c5ccccc5)c4)cc3)cc2)cc1]

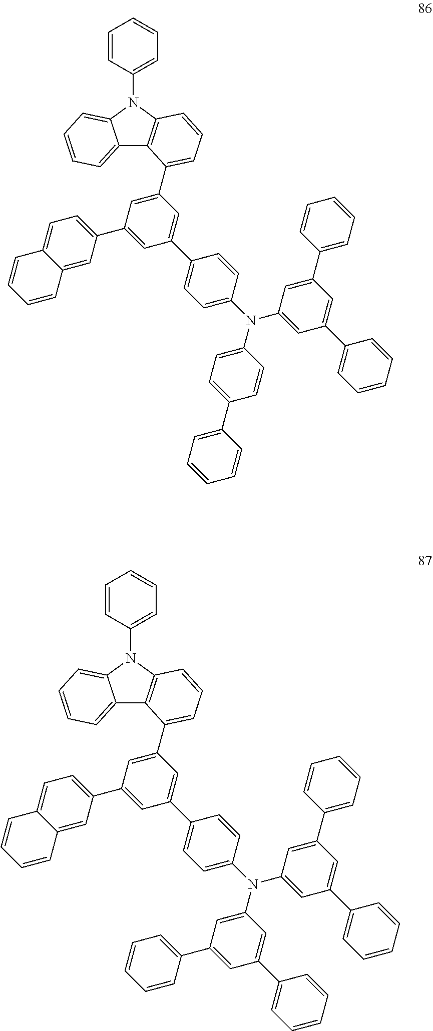 [SMILES: c1ccc(-c2ccc(N(c3ccc(-c4cc(-c5ccc6ccccc6c5)cc(-c5cccc6c5c5ccccc5n6-c5ccccc5)c4)cc3)c3cc(-c4ccccc4)cc(-c4ccccc4)c3)cc2)cc1 ; c1ccc(-c2cc(-c3ccccc3)cc(N(c3ccc(-c4cc(-c5ccc6ccccc6c5)cc(-c5cccc6c5c5ccccc5n6-c5ccccc5)c4)cc3)c3cc(-c4ccccc4)cc(-c4ccccc4)c3)c2)cc1]

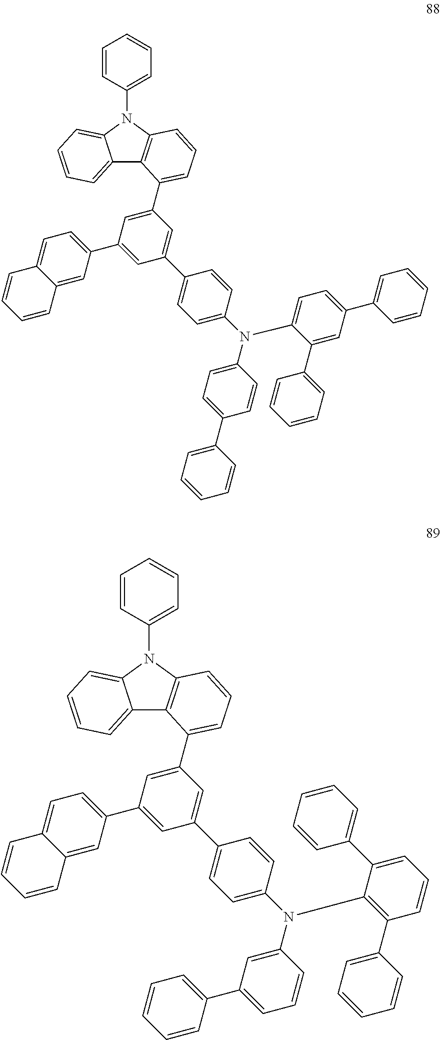 [SMILES: c1ccc(-c2ccc(N(c3ccc(-c4cc(-c5ccc6ccccc6c5)cc(-c5cccc6c5c5ccccc5n6-c5ccccc5)c4)cc3)c3ccc(-c4ccccc4)cc3-c3ccccc3)cc2)cc1 ; c1ccc(-c2cccc(N(c3ccc(-c4cc(-c5ccc6ccccc6c5)cc(-c5cccc6c5c5ccccc5n6-c5ccccc5)c4)cc3)c3c(-c4ccccc4)cccc3-c3ccccc3)c2)cc1]

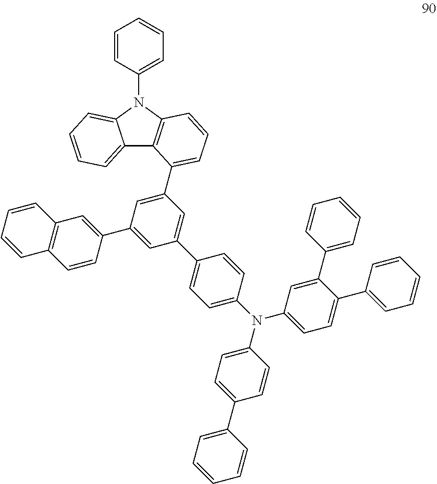 [SMILES: c1ccc(-c2ccc(N(c3ccc(-c4cc(-c5ccc6ccccc6c5)cc(-c5cccc6c5c5ccccc5n6-c5ccccc5)c4)cc3)c3ccc(-c4ccccc4)c(-c4ccccc4)c3)cc2)cc1]

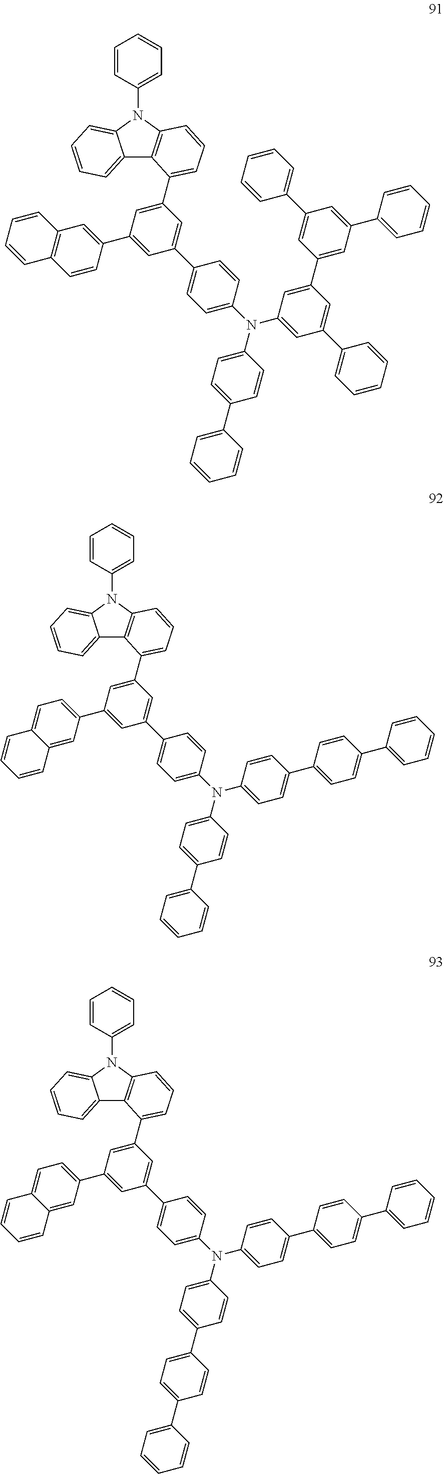 [SMILES: c1ccc(-c2ccc(N(c3ccc(-c4cc(-c5ccc6ccccc6c5)cc(-c5cccc6c5c5ccccc5n6-c5ccccc5)c4)cc3)c3cc(-c4ccccc4)cc(-c4cc(-c5ccccc5)cc(-c5ccccc5)c4)c3)cc2)cc1 ; c1ccc(-c2ccc(-c3ccc(N(c4ccc(-c5ccccc5)cc4)c4ccc(-c5cc(-c6ccc7ccccc7c6)cc(-c6cccc7c6c6ccccc6n7-c6ccccc6)c5)cc4)cc3)cc2)cc1 ; c1ccc(-c2ccc(-c3ccc(N(c4ccc(-c5ccc(-c6ccccc6)cc5)cc4)c4ccc(-c5cc(-c6ccc7ccccc7c6)cc(-c6cccc7c6c6ccccc6n7-c6ccccc6)c5)cc4)cc3)cc2)cc1]

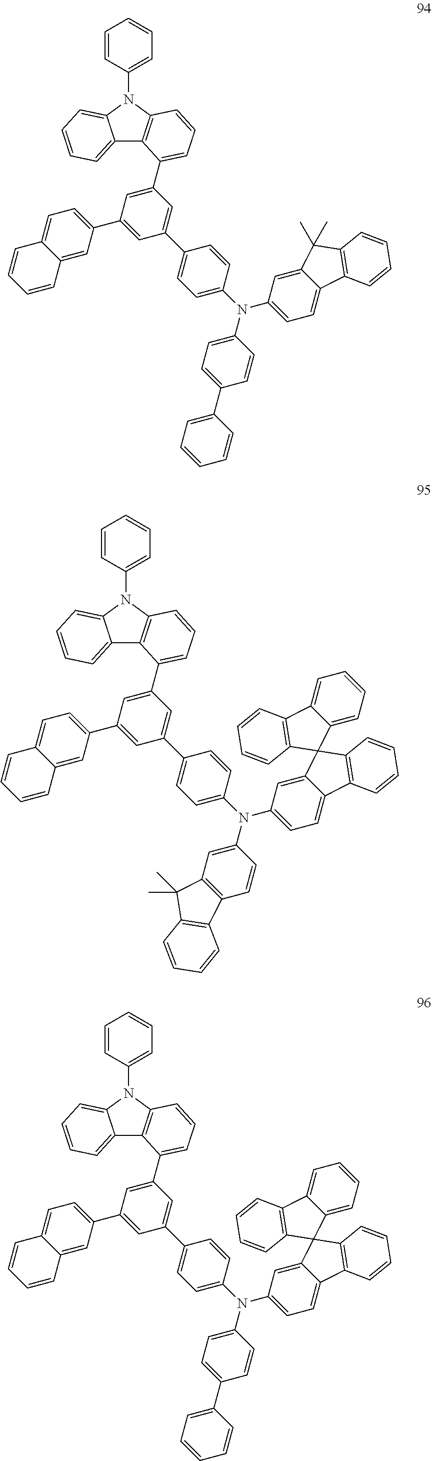 [SMILES: CC1(C)c2ccccc2-c2ccc(N(c3ccc(-c4ccccc4)cc3)c3ccc(-c4cc(-c5ccc6ccccc6c5)cc(-c5cccc6c5c5ccccc5n6-c5ccccc5)c4)cc3)cc21 ; CC1(C)c2ccccc2-c2ccc(N(c3ccc(-c4cc(-c5ccc6ccccc6c5)cc(-c5cccc6c5c5ccccc5n6-c5ccccc5)c4)cc3)c3ccc4c(c3)C3(c5ccccc5-c5ccccc53)c3ccccc3-4)cc21 ; c1ccc(-c2ccc(N(c3ccc(-c4cc(-c5ccc6ccccc6c5)cc(-c5cccc6c5c5ccccc5n6-c5ccccc5)c4)cc3)c3ccc4c(c3)C3(c5ccccc5-c5ccccc53)c3ccccc3-4)cc2)cc1]

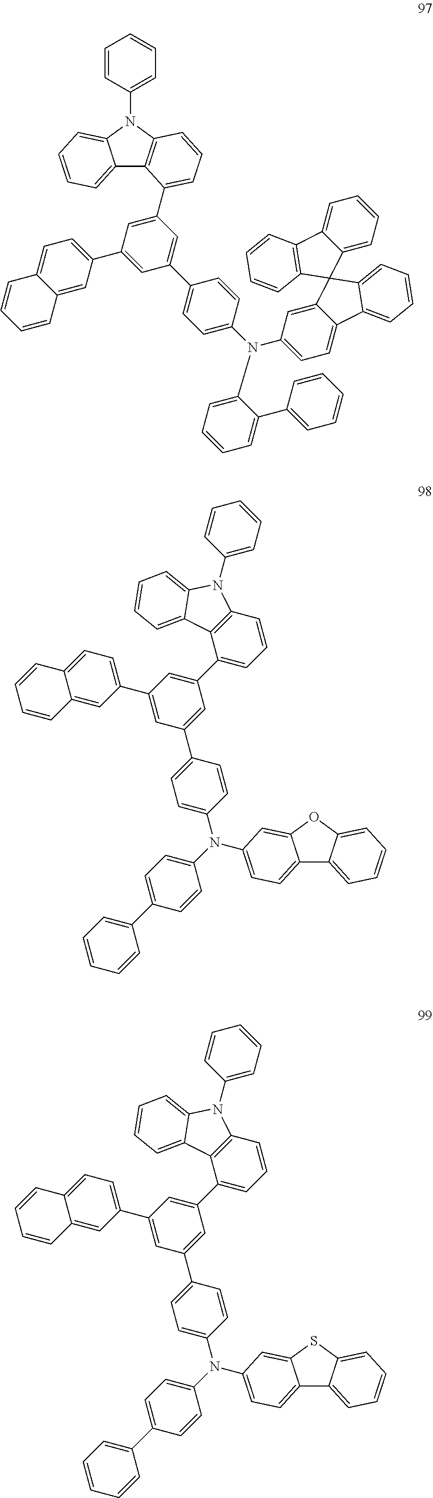 [SMILES: c1ccc(-c2ccccc2N(c2ccc(-c3cc(-c4ccc5ccccc5c4)cc(-c4cccc5c4c4ccccc4n5-c4ccccc4)c3)cc2)c2ccc3c(c2)C2(c4ccccc4-c4ccccc42)c2ccccc2-3)cc1 ; c1ccc(-c2ccc(N(c3ccc(-c4cc(-c5ccc6ccccc6c5)cc(-c5cccc6c5c5ccccc5n6-c5ccccc5)c4)cc3)c3ccc4c(c3)oc3ccccc34)cc2)cc1 ; c1ccc(-c2ccc(N(c3ccc(-c4cc(-c5ccc6ccccc6c5)cc(-c5cccc6c5c5ccccc5n6-c5ccccc5)c4)cc3)c3ccc4c(c3)sc3ccccc34)cc2)cc1]

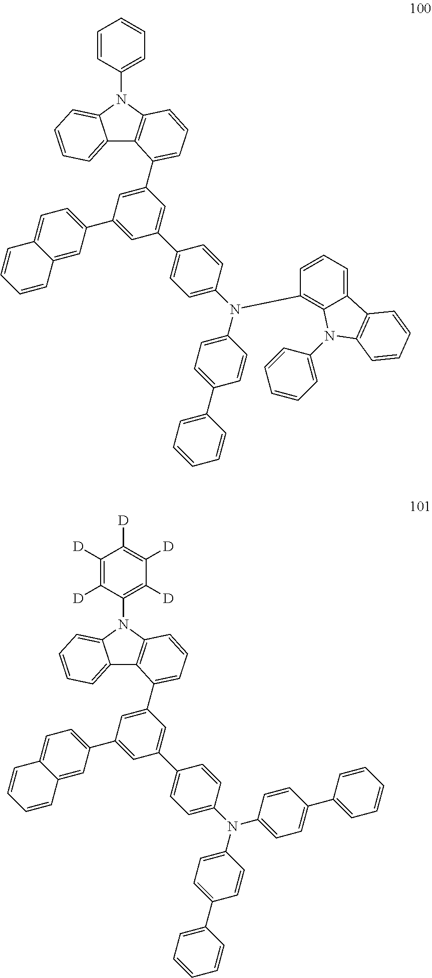 [SMILES: c1ccc(-c2ccc(N(c3ccc(-c4cc(-c5ccc6ccccc6c5)cc(-c5cccc6c5c5ccccc5n6-c5ccccc5)c4)cc3)c3cccc4c5ccccc5n(-c5ccccc5)c34)cc2)cc1 ; [2H]c1c([2H])c([2H])c(-n2c3ccccc3c3c(-c4cc(-c5ccc(N(c6ccc(-c7ccccc7)cc6)c6ccc(-c7ccccc7)cc6)cc5)cc(-c5ccc6ccccc6c5)c4)cccc32)c([2H])c1[2H]]

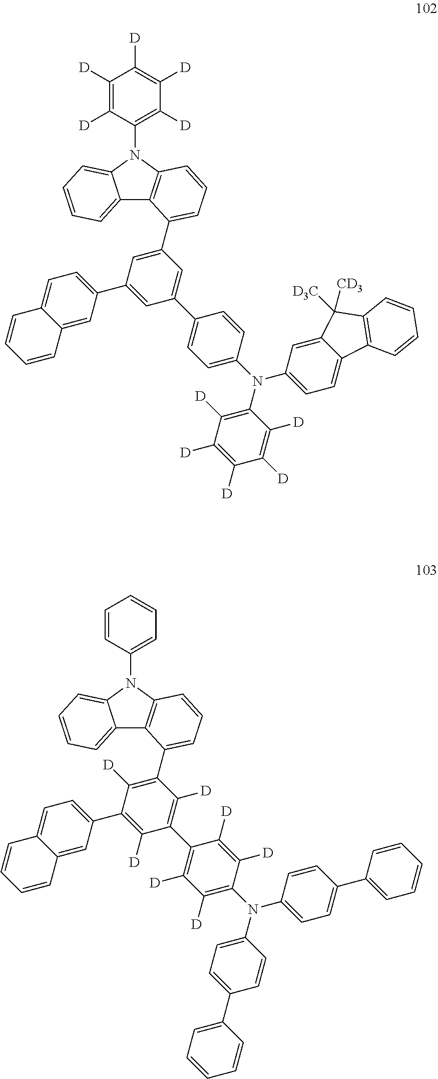 [SMILES: [2H]c1c([2H])c([2H])c(N(c2ccc(-c3cc(-c4ccc5ccccc5c4)cc(-c4cccc5c4c4ccccc4n5-c4c([2H])c([2H])c([2H])c([2H])c4[2H])c3)cc2)c2ccc3c(c2)C(C([2H])([2H])[2H])(C([2H])([2H])[2H])c2ccccc2-3)c([2H])c1[2H] ; [2H]c1c([2H])c(N(c2ccc(-c3ccccc3)cc2)c2ccc(-c3ccccc3)cc2)c([2H])c([2H])c1-c1c([2H])c(-c2ccc3ccccc3c2)c([2H])c(-c2cccc3c2c2ccccc2n3-c2ccccc2)c1[2H]]

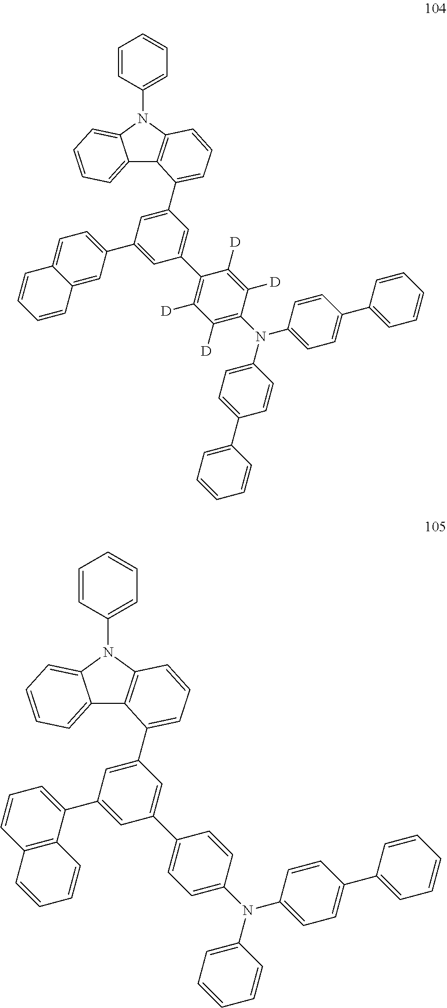 [SMILES: [2H]c1c([2H])c(N(c2ccc(-c3ccccc3)cc2)c2ccc(-c3ccccc3)cc2)c([2H])c([2H])c1-c1cc(-c2ccc3ccccc3c2)cc(-c2cccc3c2c2ccccc2n3-c2ccccc2)c1 ; c1ccc(-c2ccc(N(c3ccccc3)c3ccc(-c4cc(-c5cccc6ccccc56)cc(-c5cccc6c5c5ccccc5n6-c5ccccc5)c4)cc3)cc2)cc1]

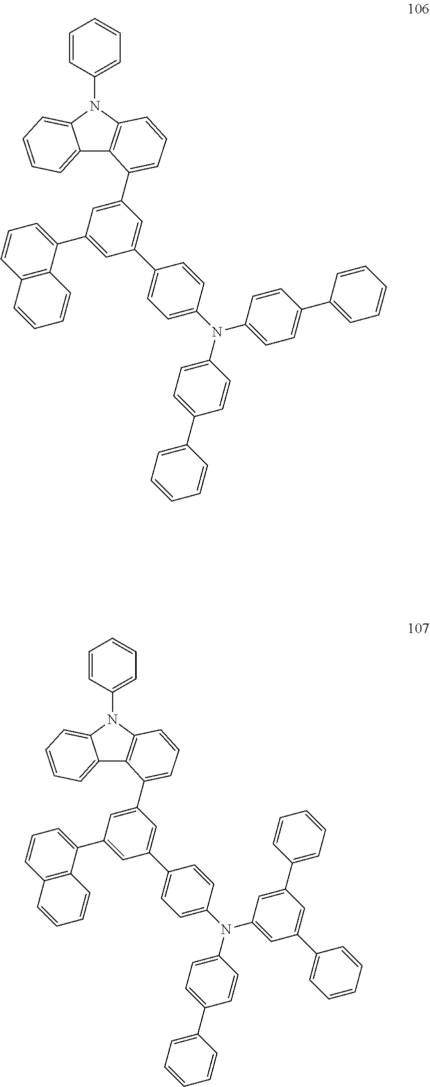 [SMILES: c1ccc(-c2ccc(N(c3ccc(-c4ccccc4)cc3)c3ccc(-c4cc(-c5cccc6ccccc56)cc(-c5cccc6c5c5ccccc5n6-c5ccccc5)c4)cc3)cc2)cc1 ; c1ccc(-c2ccc(N(c3ccc(-c4cc(-c5cccc6ccccc56)cc(-c5cccc6c5c5ccccc5n6-c5ccccc5)c4)cc3)c3cc(-c4ccccc4)cc(-c4ccccc4)c3)cc2)cc1]

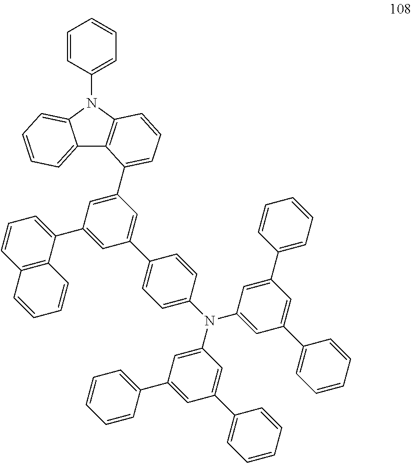 [SMILES: c1ccc(-c2cc(-c3ccccc3)cc(N(c3ccc(-c4cc(-c5cccc6ccccc56)cc(-c5cccc6c5c5ccccc5n6-c5ccccc5)c4)cc3)c3cc(-c4ccccc4)cc(-c4ccccc4)c3)c2)cc1]

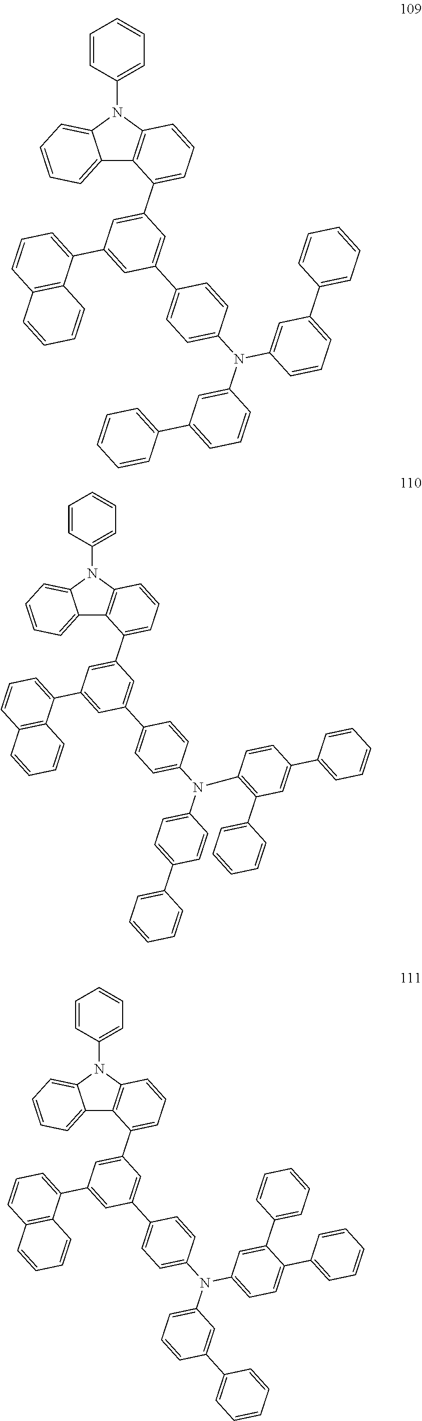

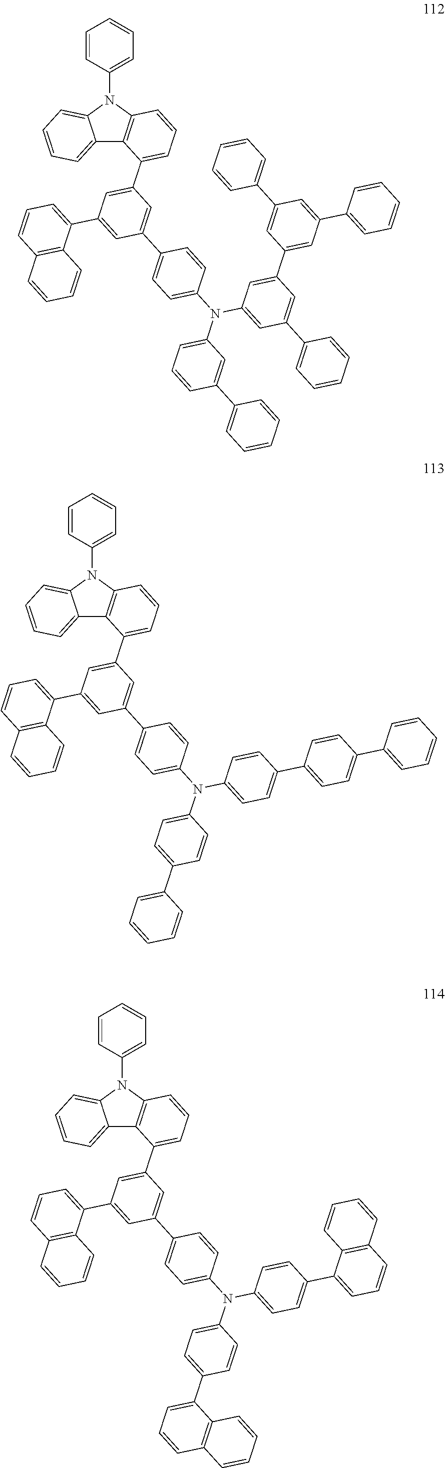 [SMILES: c1ccc(-c2cc(-c3ccccc3)cc(-c3cc(-c4ccccc4)cc(N(c4ccc(-c5cc(-c6cccc7ccccc67)cc(-c6cccc7c6c6ccccc6n7-c6ccccc6)c5)cc4)c4cccc(-c5ccccc5)c4)c3)c2)cc1 ; c1ccc(-c2ccc(-c3ccc(N(c4ccc(-c5ccccc5)cc4)c4ccc(-c5cc(-c6cccc7ccccc67)cc(-c6cccc7c6c6ccccc6n7-c6ccccc6)c5)cc4)cc3)cc2)cc1 ; c1ccc(-n2c3ccccc3c3c(-c4cc(-c5ccc(N(c6ccc(-c7cccc8ccccc78)cc6)c6ccc(-c7cccc8ccccc78)cc6)cc5)cc(-c5cccc6ccccc56)c4)cccc32)cc1]

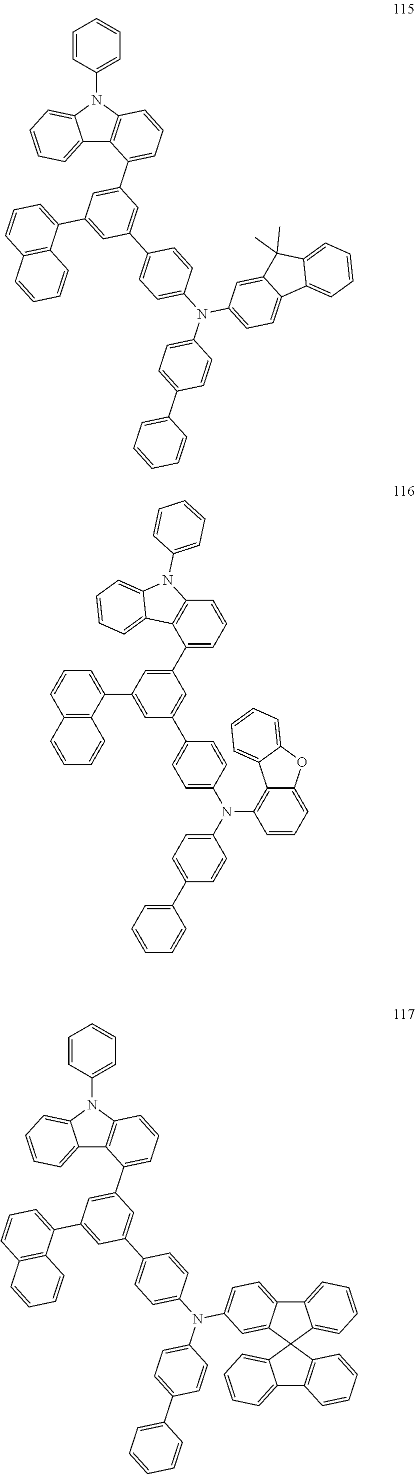 [SMILES: CC1(C)c2ccccc2-c2ccc(N(c3ccc(-c4ccccc4)cc3)c3ccc(-c4cc(-c5cccc6ccccc56)cc(-c5cccc6c5c5ccccc5n6-c5ccccc5)c4)cc3)cc21 ; c1ccc(-c2ccc(N(c3ccc(-c4cc(-c5cccc6ccccc56)cc(-c5cccc6c5c5ccccc5n6-c5ccccc5)c4)cc3)c3cccc4oc5ccccc5c34)cc2)cc1 ; c1ccc(-c2ccc(N(c3ccc(-c4cc(-c5cccc6ccccc56)cc(-c5cccc6c5c5ccccc5n6-c5ccccc5)c4)cc3)c3ccc4c(c3)C3(c5ccccc5-c5ccccc53)c3ccccc3-4)cc2)cc1]

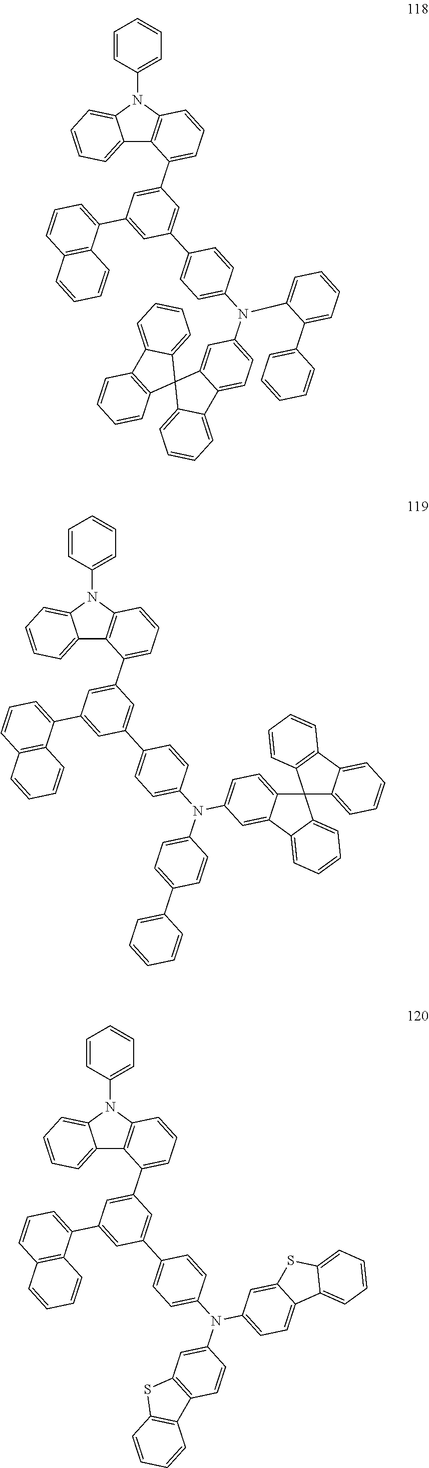 [SMILES: c1ccc(-c2ccccc2N(c2ccc(-c3cc(-c4cccc5ccccc45)cc(-c4cccc5c4c4ccccc4n5-c4ccccc4)c3)cc2)c2ccc3c(c2)C2(c4ccccc4-c4ccccc42)c2ccccc2-3)cc1 ; c1ccc(-c2ccc(N(c3ccc(-c4cc(-c5cccc6ccccc56)cc(-c5cccc6c5c5ccccc5n6-c5ccccc5)c4)cc3)c3ccc4c(c3)-c3ccccc3C43c4ccccc4-c4ccccc43)cc2)cc1 ; c1ccc(-n2c3ccccc3c3c(-c4cc(-c5ccc(N(c6ccc7c(c6)sc6ccccc67)c6ccc7c(c6)sc6ccccc67)cc5)cc(-c5cccc6ccccc56)c4)cccc32)cc1]

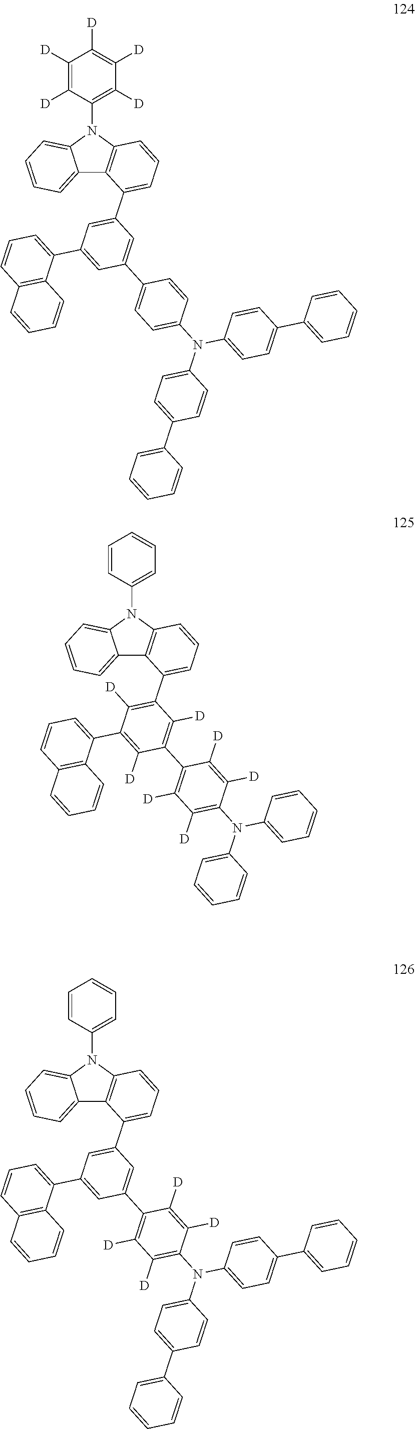

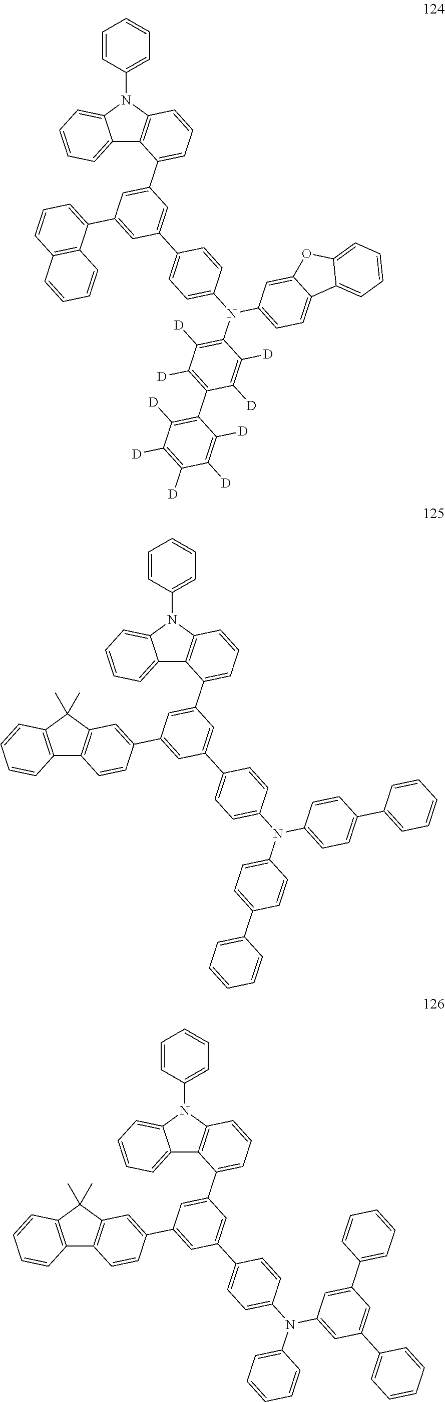 [SMILES: [2H]c1c([2H])c([2H])c(-c2c([2H])c([2H])c(N(c3ccc(-c4cc(-c5cccc6ccccc56)cc(-c5cccc6c5c5ccccc5n6-c5ccccc5)c4)cc3)c3ccc4c(c3)oc3ccccc34)c([2H])c2[2H])c([2H])c1[2H] ; CC1(C)c2ccccc2-c2ccc(-c3cc(-c4ccc(N(c5ccc(-c6ccccc6)cc5)c5ccc(-c6ccccc6)cc5)cc4)cc(-c4cccc5c4c4ccccc4n5-c4ccccc4)c3)cc21 ; CC1(C)c2ccccc2-c2ccc(-c3cc(-c4ccc(N(c5ccccc5)c5cc(-c6ccccc6)cc(-c6ccccc6)c5)cc4)cc(-c4cccc5c4c4ccccc4n5-c4ccccc4)c3)cc21]

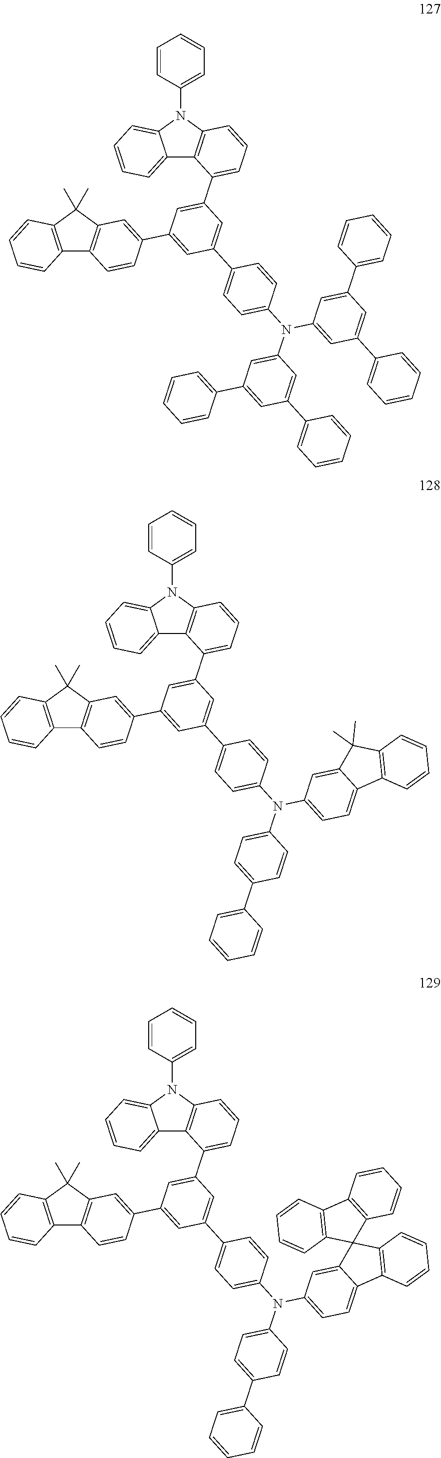 [SMILES: CC1(C)c2ccccc2-c2ccc(-c3cc(-c4ccc(N(c5cc(-c6ccccc6)cc(-c6ccccc6)c5)c5cc(-c6ccccc6)cc(-c6ccccc6)c5)cc4)cc(-c4cccc5c4c4ccccc4n5-c4ccccc4)c3)cc21 ; CC1(C)c2ccccc2-c2ccc(-c3cc(-c4ccc(N(c5ccc(-c6ccccc6)cc5)c5ccc6c(c5)C(C)(C)c5ccccc5-6)cc4)cc(-c4cccc5c4c4ccccc4n5-c4ccccc4)c3)cc21 ; CC1(C)c2ccccc2-c2ccc(-c3cc(-c4ccc(N(c5ccc(-c6ccccc6)cc5)c5ccc6c(c5)C5(c7ccccc7-c7ccccc75)c5ccccc5-6)cc4)cc(-c4cccc5c4c4ccccc4n5-c4ccccc4)c3)cc21]

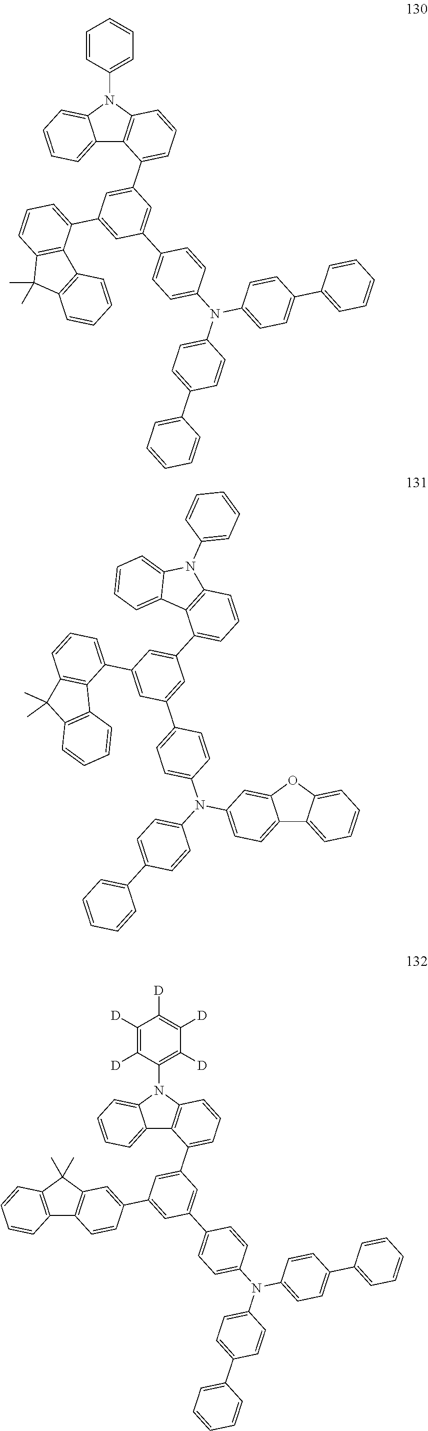

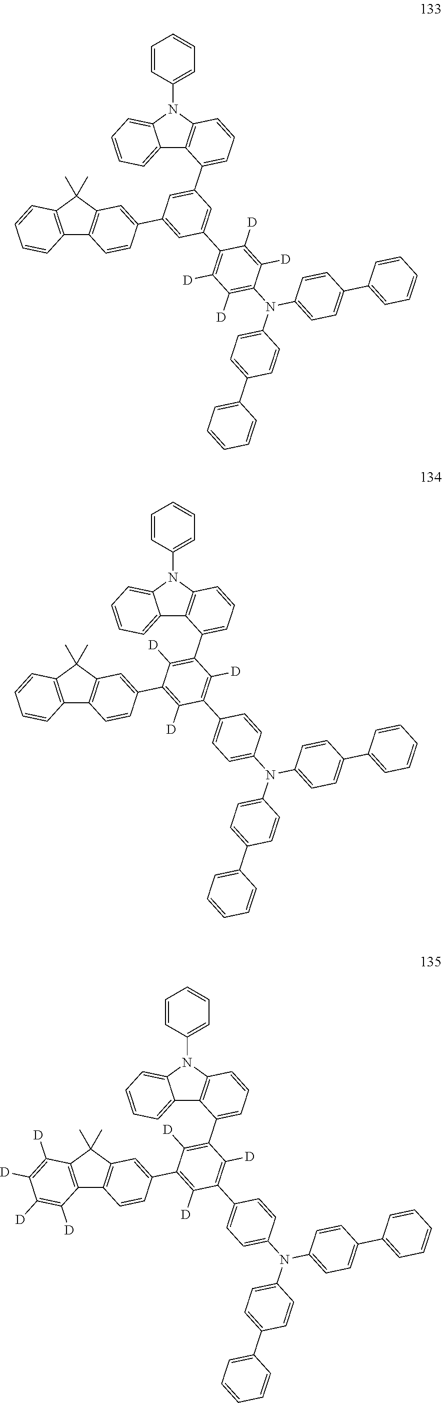 [SMILES: [2H]c1c([2H])c(N(c2ccc(-c3ccccc3)cc2)c2ccc(-c3ccccc3)cc2)c([2H])c([2H])c1-c1cc(-c2ccc3c(c2)C(C)(C)c2ccccc2-3)cc(-c2cccc3c2c2ccccc2n3-c2ccccc2)c1 ; [2H]c1c(-c2ccc(N(c3ccc(-c4ccccc4)cc3)c3ccc(-c4ccccc4)cc3)cc2)c([2H])c(-c2cccc3c2c2ccccc2n3-c2ccccc2)c([2H])c1-c1ccc2c(c1)C(C)(C)c1ccccc1-2 ; [2H]c1c([2H])c([2H])c2c(c1[2H])-c1ccc(-c3c([2H])c(-c4ccc(N(c5ccc(-c6ccccc6)cc5)c5ccc(-c6ccccc6)cc5)cc4)c([2H])c(-c4cccc5c4c4ccccc4n5-c4ccccc4)c3[2H])cc1C2(C)C]

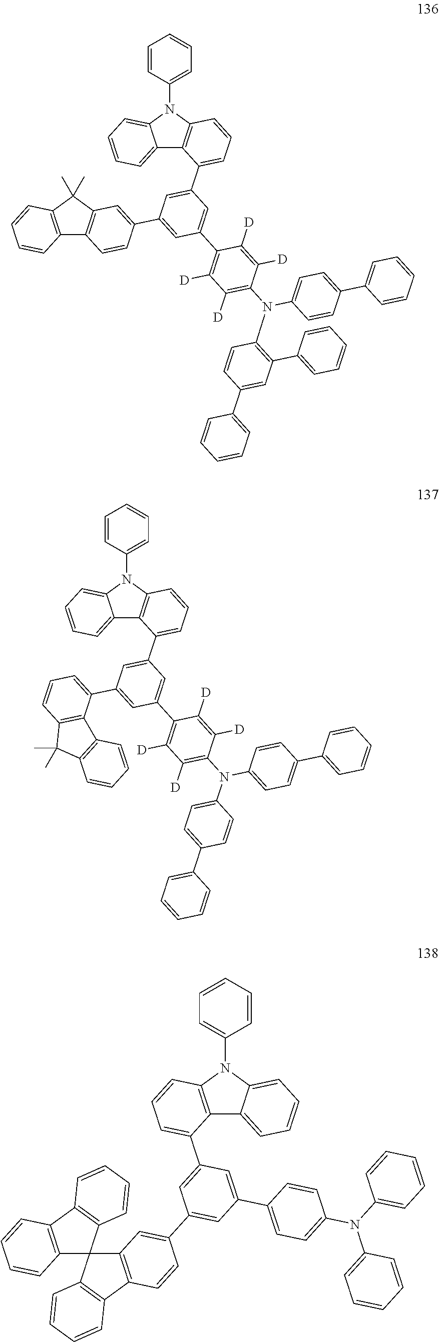 [SMILES: [2H]c1c([2H])c(N(c2ccc(-c3ccccc3)cc2)c2ccc(-c3ccccc3)cc2-c2ccccc2)c([2H])c([2H])c1-c1cc(-c2ccc3c(c2)C(C)(C)c2ccccc2-3)cc(-c2cccc3c2c2ccccc2n3-c2ccccc2)c1 ; [2H]c1c([2H])c(N(c2ccc(-c3ccccc3)cc2)c2ccc(-c3ccccc3)cc2)c([2H])c([2H])c1-c1cc(-c2cccc3c2-c2ccccc2C3(C)C)cc(-c2cccc3c2c2ccccc2n3-c2ccccc2)c1 ; c1ccc(N(c2ccccc2)c2ccc(-c3cc(-c4ccc5c(c4)C4(c6ccccc6-c6ccccc64)c4ccccc4-5)cc(-c4cccc5c4c4ccccc4n5-c4ccccc4)c3)cc2)cc1]

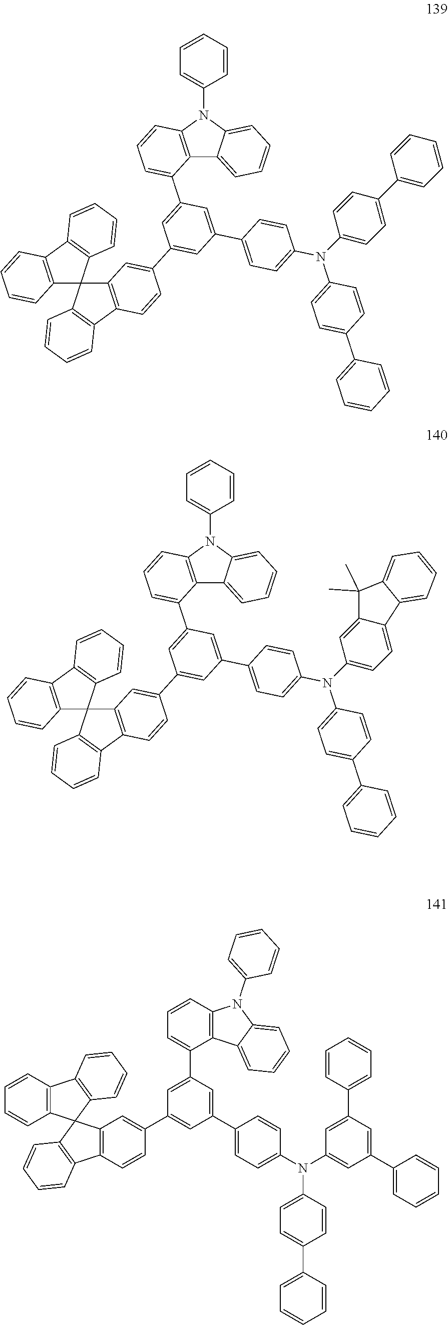 [SMILES: c1ccc(-c2ccc(N(c3ccc(-c4ccccc4)cc3)c3ccc(-c4cc(-c5ccc6c(c5)C5(c7ccccc7-c7ccccc75)c5ccccc5-6)cc(-c5cccc6c5c5ccccc5n6-c5ccccc5)c4)cc3)cc2)cc1 ; CC1(C)c2ccccc2-c2ccc(N(c3ccc(-c4ccccc4)cc3)c3ccc(-c4cc(-c5ccc6c(c5)C5(c7ccccc7-c7ccccc75)c5ccccc5-6)cc(-c5cccc6c5c5ccccc5n6-c5ccccc5)c4)cc3)cc21 ; c1ccc(-c2ccc(N(c3ccc(-c4cc(-c5ccc6c(c5)C5(c7ccccc7-c7ccccc75)c5ccccc5-6)cc(-c5cccc6c5c5ccccc5n6-c5ccccc5)c4)cc3)c3cc(-c4ccccc4)cc(-c4ccccc4)c3)cc2)cc1]

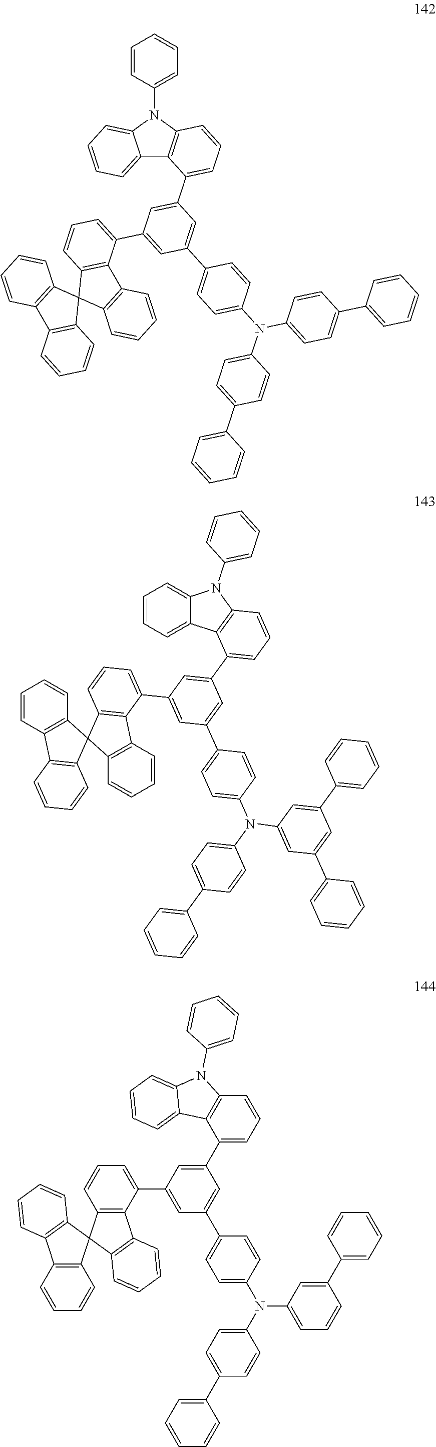

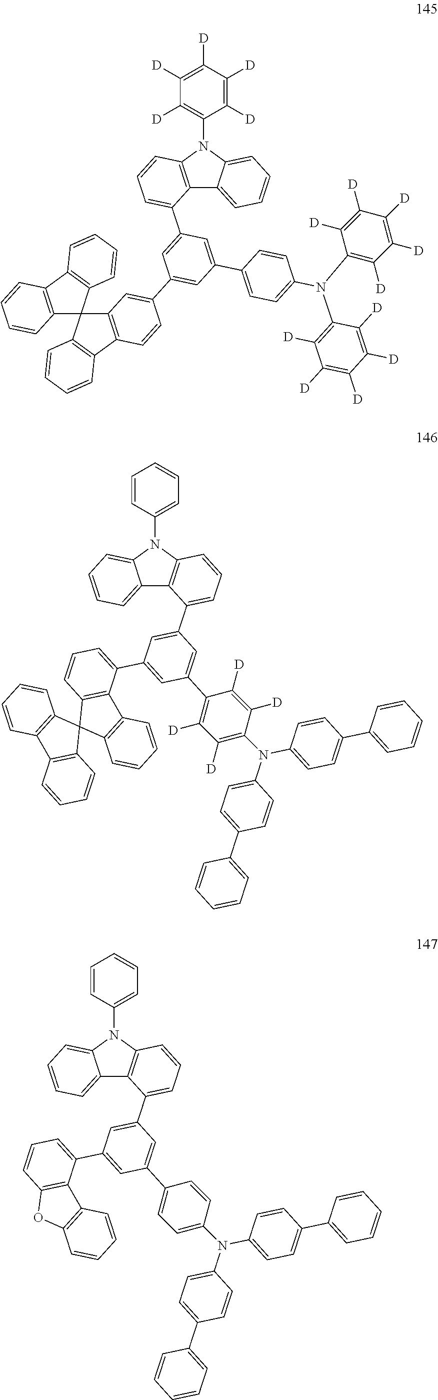 [SMILES: [2H]c1c([2H])c([2H])c(N(c2ccc(-c3cc(-c4ccc5c(c4)C4(c6ccccc6-c6ccccc64)c4ccccc4-5)cc(-c4cccc5c4c4ccccc4n5-c4c([2H])c([2H])c([2H])c([2H])c4[2H])c3)cc2)c2c([2H])c([2H])c([2H])c([2H])c2[2H])c([2H])c1[2H] ; [2H]c1c([2H])c(N(c2ccc(-c3ccccc3)cc2)c2ccc(-c3ccccc3)cc2)c([2H])c([2H])c1-c1cc(-c2cccc3c2-c2ccccc2C32c3ccccc3-c3ccccc32)cc(-c2cccc3c2c2ccccc2n3-c2ccccc2)c1 ; c1ccc(-c2ccc(N(c3ccc(-c4ccccc4)cc3)c3ccc(-c4cc(-c5cccc6oc7ccccc7c56)cc(-c5cccc6c5c5ccccc5n6-c5ccccc5)c4)cc3)cc2)cc1]

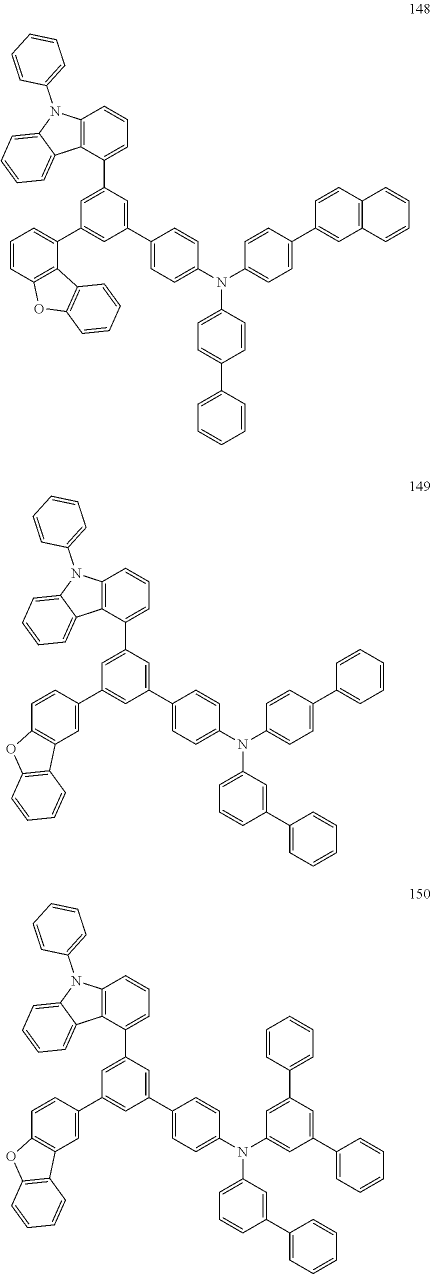 [SMILES: c1ccc(-c2ccc(N(c3ccc(-c4cc(-c5cccc6oc7ccccc7c56)cc(-c5cccc6c5c5ccccc5n6-c5ccccc5)c4)cc3)c3ccc(-c4ccc5ccccc5c4)cc3)cc2)cc1 ; c1ccc(-c2ccc(N(c3ccc(-c4cc(-c5ccc6oc7ccccc7c6c5)cc(-c5cccc6c5c5ccccc5n6-c5ccccc5)c4)cc3)c3cccc(-c4ccccc4)c3)cc2)cc1 ; c1ccc(-c2cccc(N(c3ccc(-c4cc(-c5ccc6oc7ccccc7c6c5)cc(-c5cccc6c5c5ccccc5n6-c5ccccc5)c4)cc3)c3cc(-c4ccccc4)cc(-c4ccccc4)c3)c2)cc1]

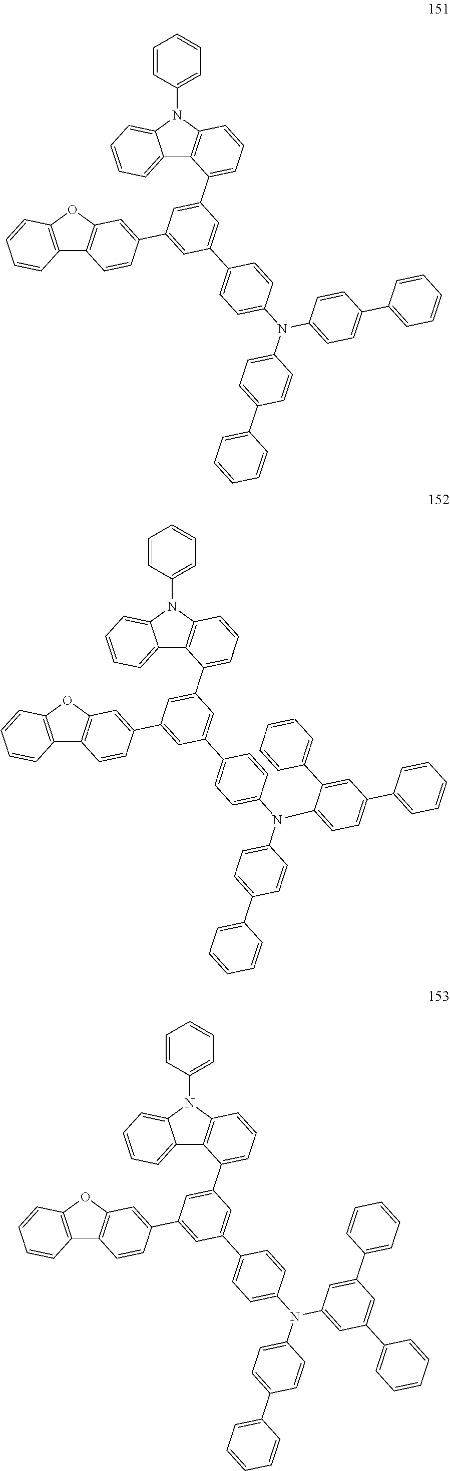 [SMILES: c1ccc(-c2ccc(N(c3ccc(-c4ccccc4)cc3)c3ccc(-c4cc(-c5ccc6c(c5)oc5ccccc56)cc(-c5cccc6c5c5ccccc5n6-c5ccccc5)c4)cc3)cc2)cc1 ; c1ccc(-c2ccc(N(c3ccc(-c4cc(-c5ccc6c(c5)oc5ccccc56)cc(-c5cccc6c5c5ccccc5n6-c5ccccc5)c4)cc3)c3ccc(-c4ccccc4)cc3-c3ccccc3)cc2)cc1 ; c1ccc(-c2ccc(N(c3ccc(-c4cc(-c5ccc6c(c5)oc5ccccc56)cc(-c5cccc6c5c5ccccc5n6-c5ccccc5)c4)cc3)c3cc(-c4ccccc4)cc(-c4ccccc4)c3)cc2)cc1]

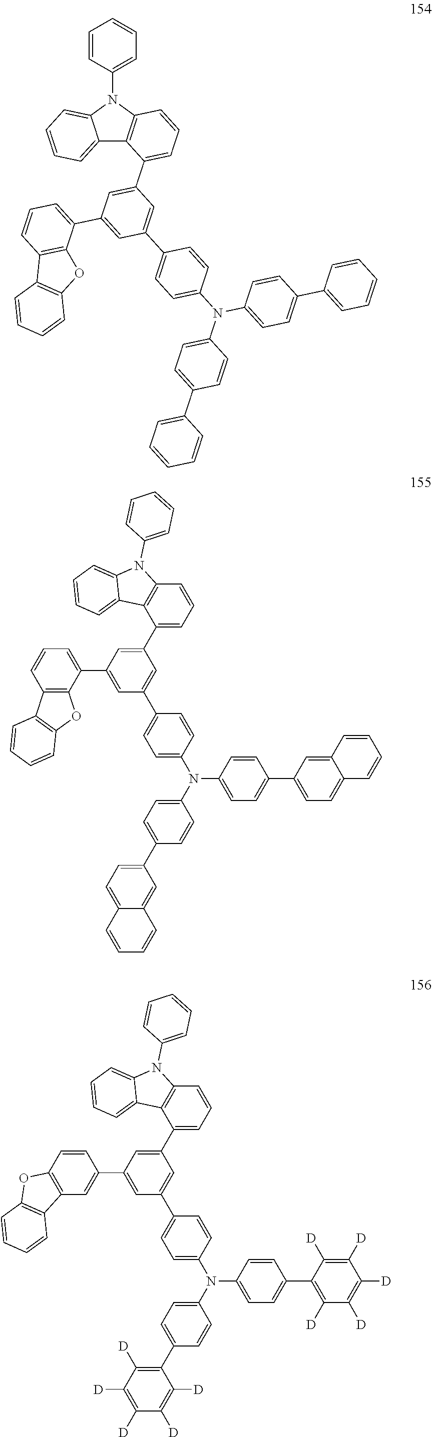

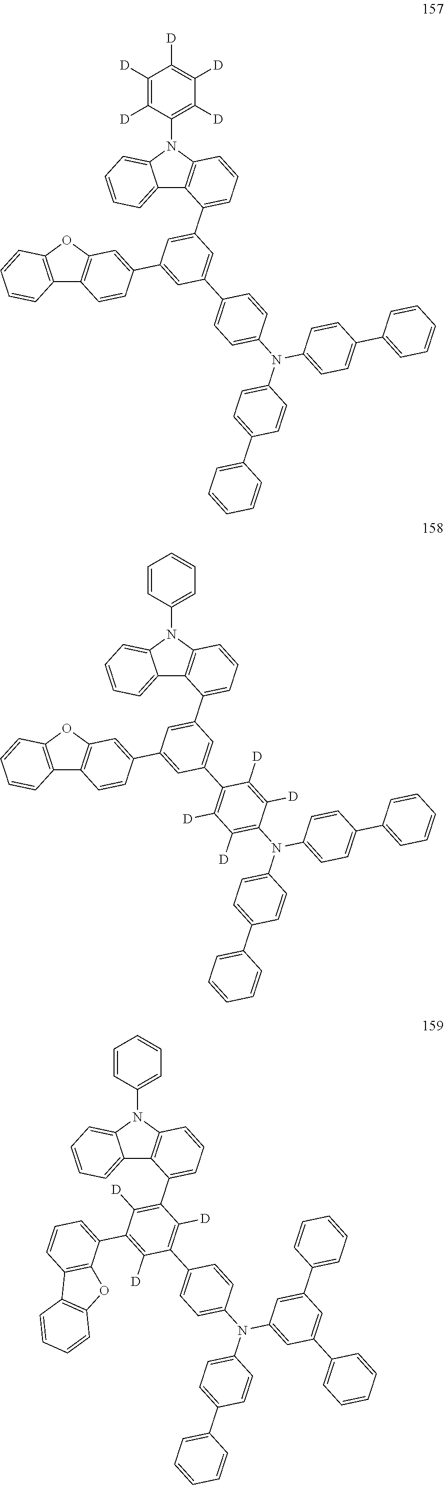 [SMILES: [2H]c1c([2H])c([2H])c(-n2c3ccccc3c3c(-c4cc(-c5ccc(N(c6ccc(-c7ccccc7)cc6)c6ccc(-c7ccccc7)cc6)cc5)cc(-c5ccc6c(c5)oc5ccccc56)c4)cccc32)c([2H])c1[2H] ; [2H]c1c([2H])c(N(c2ccc(-c3ccccc3)cc2)c2ccc(-c3ccccc3)cc2)c([2H])c([2H])c1-c1cc(-c2ccc3c(c2)oc2ccccc23)cc(-c2cccc3c2c2ccccc2n3-c2ccccc2)c1 ; [2H]c1c(-c2ccc(N(c3ccc(-c4ccccc4)cc3)c3cc(-c4ccccc4)cc(-c4ccccc4)c3)cc2)c([2H])c(-c2cccc3c2c2ccccc2n3-c2ccccc2)c([2H])c1-c1cccc2c1oc1ccccc12]

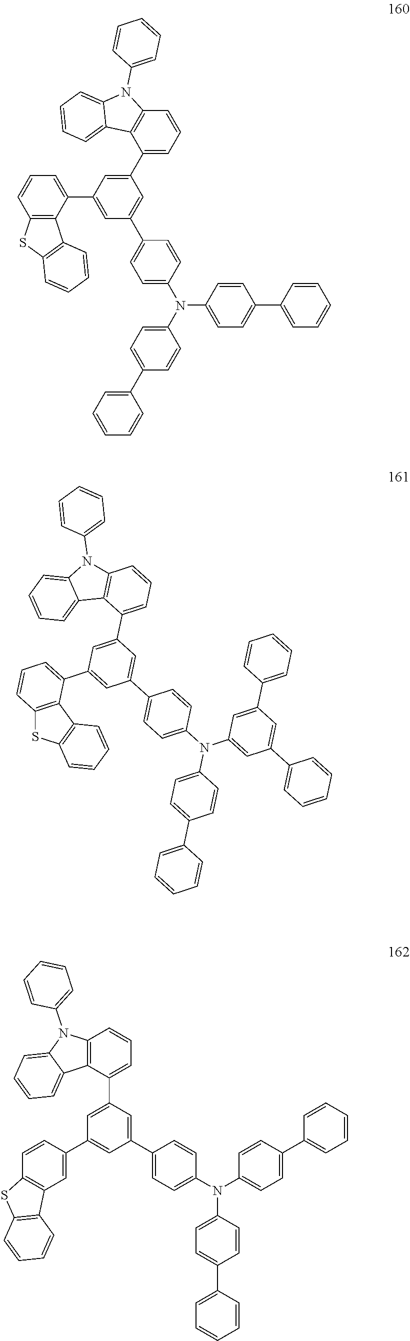 [SMILES: c1ccc(-c2ccc(N(c3ccc(-c4ccccc4)cc3)c3ccc(-c4cc(-c5cccc6sc7ccccc7c56)cc(-c5cccc6c5c5ccccc5n6-c5ccccc5)c4)cc3)cc2)cc1 ; c1ccc(-c2ccc(N(c3ccc(-c4cc(-c5cccc6sc7ccccc7c56)cc(-c5cccc6c5c5ccccc5n6-c5ccccc5)c4)cc3)c3cc(-c4ccccc4)cc(-c4ccccc4)c3)cc2)cc1 ; c1ccc(-c2ccc(N(c3ccc(-c4ccccc4)cc3)c3ccc(-c4cc(-c5ccc6sc7ccccc7c6c5)cc(-c5cccc6c5c5ccccc5n6-c5ccccc5)c4)cc3)cc2)cc1]

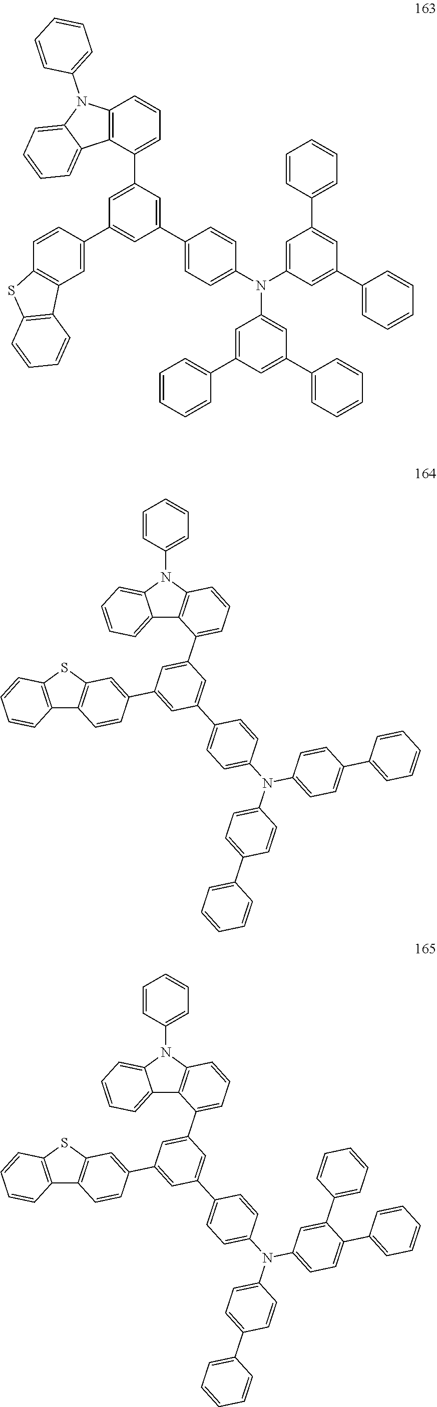 [SMILES: c1ccc(-c2cc(-c3ccccc3)cc(N(c3ccc(-c4cc(-c5ccc6sc7ccccc7c6c5)cc(-c5cccc6c5c5ccccc5n6-c5ccccc5)c4)cc3)c3cc(-c4ccccc4)cc(-c4ccccc4)c3)c2)cc1 ; c1ccc(-c2ccc(N(c3ccc(-c4ccccc4)cc3)c3ccc(-c4cc(-c5ccc6c(c5)sc5ccccc56)cc(-c5cccc6c5c5ccccc5n6-c5ccccc5)c4)cc3)cc2)cc1 ; c1ccc(-c2ccc(N(c3ccc(-c4cc(-c5ccc6c(c5)sc5ccccc56)cc(-c5cccc6c5c5ccccc5n6-c5ccccc5)c4)cc3)c3ccc(-c4ccccc4)c(-c4ccccc4)c3)cc2)cc1]

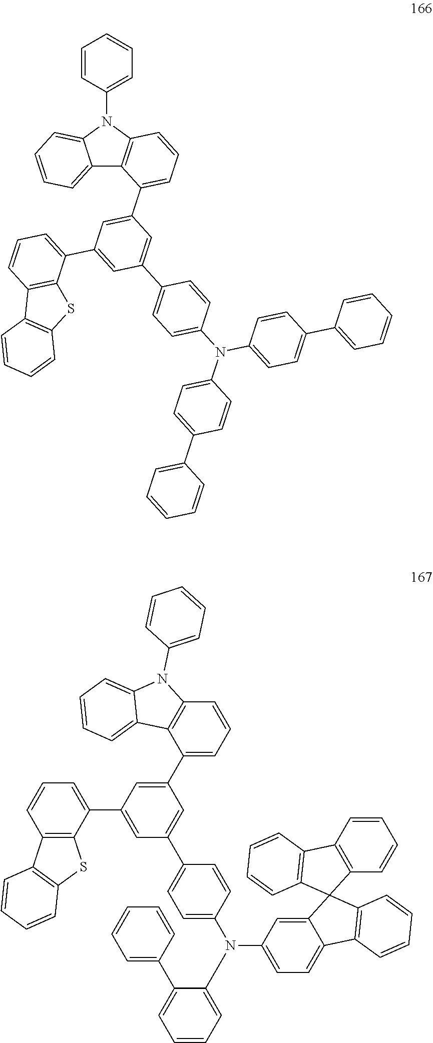 [SMILES: c1ccc(-c2ccc(N(c3ccc(-c4ccccc4)cc3)c3ccc(-c4cc(-c5cccc6c5sc5ccccc56)cc(-c5cccc6c5c5ccccc5n6-c5ccccc5)c4)cc3)cc2)cc1 ; c1ccc(-c2ccccc2N(c2ccc(-c3cc(-c4cccc5c4sc4ccccc45)cc(-c4cccc5c4c4ccccc4n5-c4ccccc4)c3)cc2)c2ccc3c(c2)C2(c4ccccc4-c4ccccc42)c2ccccc2-3)cc1]

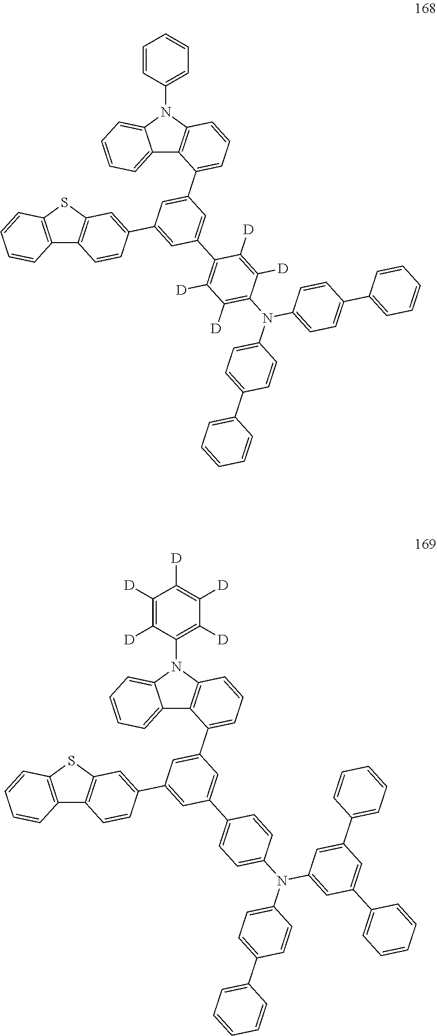 [SMILES: [2H]c1c([2H])c(N(c2ccc(-c3ccccc3)cc2)c2ccc(-c3ccccc3)cc2)c([2H])c([2H])c1-c1cc(-c2ccc3c(c2)sc2ccccc23)cc(-c2cccc3c2c2ccccc2n3-c2ccccc2)c1 ; [2H]c1c([2H])c([2H])c(-n2c3ccccc3c3c(-c4cc(-c5ccc(N(c6ccc(-c7ccccc7)cc6)c6cc(-c7ccccc7)cc(-c7ccccc7)c6)cc5)cc(-c5ccc6c(c5)sc5ccccc56)c4)cccc32)c([2H])c1[2H]]

Through the characteristic backbone structure and substituents as described above, an organic compound having the inherent characteristics of the backbone structure and the substituents may be synthesized, and for example, when an organic light-emitting device is manufactured, it is possible to manufacture an organic light-emitting compound material that satisfies the conditions required by each organic layer such as a hole transport layer and an electron blocking layer, and in particular, when the compound of [Formula I] according to the present invention is used in an electron blocking layer, a hole transport layer, and the like, the device characteristics such as the luminous efficiency of the device may be further improved.

The organic light-emitting compound according to the present invention may be used and applied to an organic light-emitting device by a typical manufacturing method.

The organic light emitting device according to an exemplary embodiment of the present invention may be composed of a structure including a first electrode, a second electrode and an organic layer disposed therebetween, and may be manufactured using typical device manufacturing methods and materials, except that the organic compound according to the present invention is used in an organic layer of the device.

The organic layer of the organic light-emitting device according to the present invention may be composed of a single-layered structure, but may also be composed of a multi-layered structure in which two or more organic layers are stacked. For example, the organic layer may have a structure including a hole injecting layer, a hole transport layer, a light emitting layer, an electron transport layer, an electron injecting layer, an electron blocking layer, and the like. However, the structure of the organic layer is not limited thereto, and may include a fewer or greater number of the organic layers.

Therefore, in the organic light emitting device according to the present invention, the organic layer may include a hole transport layer or an electron blocking layer, and at least one layer of the layers may include the organic compound represented by [Formula I].

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the method as described above, an organic light emitting device may be made by sequentially depositing a negative electrode material, an organic layer, and a positive electrode material on a substrate. The organic layer may have a multi-layer structure including a hole injecting layer, a hole transport layer, a hole blocking layer, a light-emitting layer, an electron blocking layer, an electron transport layer, an electron blocking layer, and the like, but is not limited thereto and may also have a single-layer structure. In addition, the organic layer may be manufactured to include a fewer number of layers by a method such as a solvent process, for example, spin coating, dip coating, doctor blading, screen printing, inkjet printing, or a thermal transfer method instead of a deposition method, using various polymer materials.

As the anode, a materials having a high work function is usually preferred so as to facilitate the injection of holes into an organic layer. Specific examples of the positive electrode material which may be used in the present invention include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDT), polypyrrole, and polyaniline; and the like, but are not limited thereto.

As the cathode, a material having a low work function is usually preferred so as to facilitate the injection of electrons into an organic layer. Specific examples of the negative electrode material include: a metal such as magnesium, calcium, sodium, potassium, titanium, indium, yttrium, lithium, gadolinium, aluminum, silver, tin, and lead, or an alloy thereof, a multi-layer structured material, such as LiF/Al or LiO₂/Al, and the like, but are not limited thereto.

The hole injecting layer is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport layer is a material that may receive holes transported from the anode or the hole injecting layer and may transfer the holes to the light emitting layer. A material with high hole mobility is suitable. Specific examples thereof include, but are not limited to, arylamine-based organic materials, conductive polymers, and block copolymers consisting of conjugated and non-conjugated segments.

The electron blocking layer is a layer that blocks the movement of electrons and may be formed on the hole transport layer, and may be used to block the movement of electrons without affecting the transport of holes. In addition, on the electron blocking layer, the light-emitting layer may be formed, and the hole blocking layer, the electron transport layer, and the electron injecting layer may be formed.

The hole blocking layer may be used to block the movement of holes without affecting the transport of electrons, and examples of the hole blocking layer are 1,3,5-tri(1-phenyl-1H-benzo[d]imidazol-2-yl)phenyl (TPBi), 2,9-dimethyl4,7-diphenyl-1,10-phenanthroline (BCP), 4,4-bis(N-carbazolyl)-1,1′-biphenyl (CBP), 2-(4-biphenyl)-5-(4-t-butylphenyl)-1,3,4-oxadiazole (PBD), bisbenzimidazo[2,1-a:1′,2-b′]anthra[2,1,9-def:6,5,10-d′e′f′]diisoguinoline-10,21-dione (PTCBI), 4,7-diphenyl-1,10-phenanthroline (BPhen), or the like, but are not limited thereto.

The light emitting layer is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron injecting layer may be used to have high injection efficiency of electrons transferred from the cathode. Examples of such an electron injecting layer include lithium quinolate (Liq), etc., but are not limited thereto.

An electron transport material is suitably a material having high electron mobility which may proficiently accept electrons from a negative electrode and transfer the electrons to a light emitting layer. Specific examples thereof include Al complexes of 8-hydroxyquinoline, complexes including Alq₃, organic radical compounds, hydroxyflavone-metal complexes, and the like, but are not limited thereto.

The organic light emitting device according to the present disclosure may be a top-emission type, a bottom-emission type or a dual-emission type depending on the materials used.

In addition, the organic compound according to an embodiment of the present invention may perform its function even in organic electronic devices, including organic solar cells, organic photoconductors, and organic transistors, based on a similar principle to that applied to the organic light emitting device.

### MODE FOR CARRYING OUT INVENTION

Hereinafter, the present invention will be exemplified in more detail through preferred examples. However, these examples are for more specifically describing the present invention, the scope of the present invention is not limited thereto, and it will be obvious to a person with ordinary skill in the art that various changes and modifications can be made within the scope of the present invention and the scope of the technical spirit.

### Synthesis Example 1: Synthesis of Compound 4

### (1) Preparation Example 1: Synthesis of Intermediate 4-1

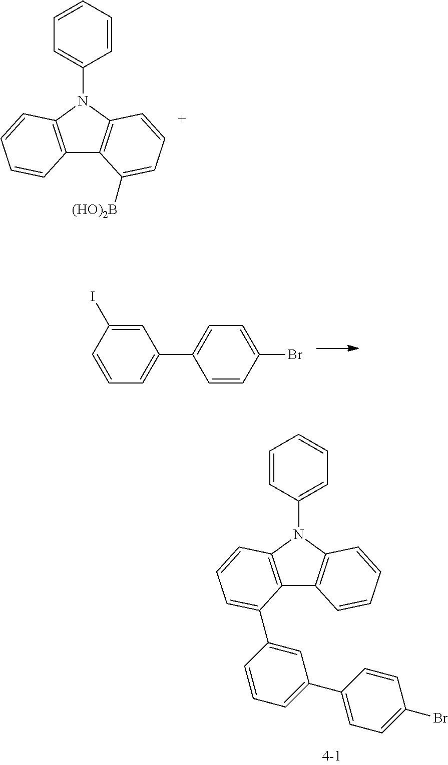 [SMILES: OB(O)c1cccc2c1c1ccccc1n2-c1ccccc1 ; Brc1ccc(-c2cccc(I)c2)cc1 ; Brc1ccc(-c2cccc(-c3cccc4c3c3ccccc3n4-c3ccccc3)c2)cc1]

200 mL of toluene, 50 mL of ethanol, and 50 mL of H₂O were added to 9-phenyl-9H-carbazol-4-yl-4-boronic acid (10.0 g, 0.035 mol), 4′-bromo-3-iodo-1,1′-biphenyl (15.0 g, 0.042 mol), K₂CO₃ (14.4 g, 0.105 mol), and Pd(PPh₃)₄ (0.8 g, 0.0007 mol), and the resulting mixture was stirred at 80° C. for 6 hours and reacted. After completion of the reaction, the resulting product was extracted and concentrated, and then subjected to column chromatography and recrystallization to obtain 11.2 g (yield 67.8%) of <Intermediate 4-1>.

### (2) Preparation Example 2: Synthesis of Compound 4

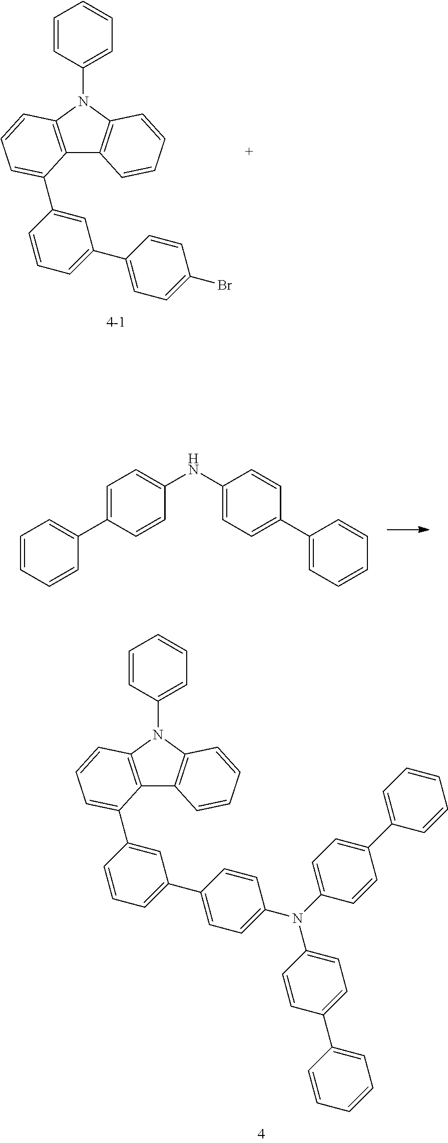 [SMILES: Brc1ccc(-c2cccc(-c3cccc4c3c3ccccc3n4-c3ccccc3)c2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cccc(-c5cccc6c5c5ccccc5n6-c5ccccc5)c4)cc3)cc2)cc1]

150 mL of xylene was added to Intermediate 4-1 (10.0 g, 0.021 mol), bis(4-biphenylyl)amine (10.2 g, 0.032 mol), NaOtBu (6.1 g, 0.063 mol), Pd(dba)₂ (0.5 g, 0.8 mmol), and t-BU₃P (0.3 g, 1.7 mmol), and the resulting mixture was stirred at 70° C. for 4 hours and reacted. After completion of the reaction, the resulting product was extracted and concentrated, and then subjected to column chromatography and recrystallization to obtain 9.6 g (yield 63.7%) of <Compound 4>.

LC/MS: m/z=714[(M)⁺]

### Synthesis Example 2: Synthesis of Compound 7

### (1) Preparation Example 1: Synthesis of Intermediate 7-1

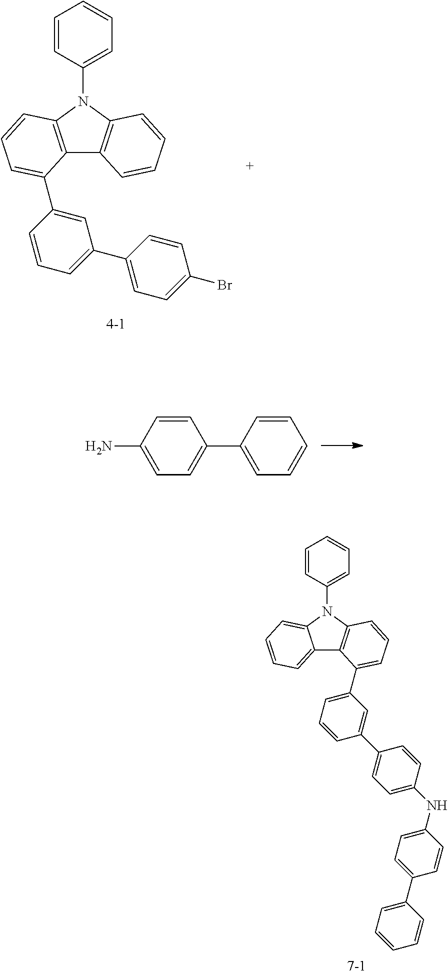 [SMILES: Brc1ccc(-c2cccc(-c3cccc4c3c3ccccc3n4-c3ccccc3)c2)cc1 ; Nc1ccc(-c2ccccc2)cc1 ; c1ccc(-c2ccc(Nc3ccc(-c4cccc(-c5cccc6c5c5ccccc5n6-c5ccccc5)c4)cc3)cc2)cc1]

150 mL of xylene was added to Intermediate 4-1 (10.0 g, 0.021 mol), 4-aminobiphenyl (5.4 g, 0.032 mol), NaOtBu (6.1 g, 0.063 mol), Pd(dba)₂ (0.5 g, 0.8 mmol), and t-BU₃P (0.3 g, 1.7 mmol), and the resulting mixture was stirred at 70° C. for 4 hours and reacted. After completion of the reaction, the resulting product was extracted and concentrated, and then subjected to column chromatography and recrystallization to obtain 8.2 g (yield 69.1%) of <Intermediate 7-1>.

### (2) Preparation Example 2. Synthesis of Compound 7

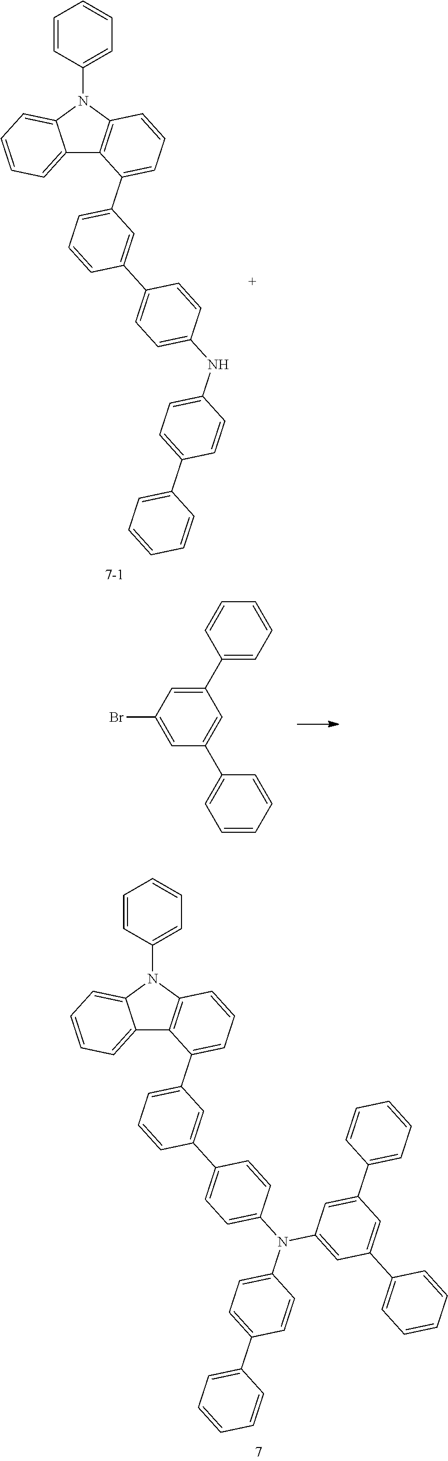 [SMILES: c1ccc(-c2ccc(Nc3ccc(-c4cccc(-c5cccc6c5c5ccccc5n6-c5ccccc5)c4)cc3)cc2)cc1 ; Brc1cc(-c2ccccc2)cc(-c2ccccc2)c1 ; c1ccc(-c2ccc(N(c3ccc(-c4cccc(-c5cccc6c5c5ccccc5n6-c5ccccc5)c4)cc3)c3cc(-c4ccccc4)cc(-c4ccccc4)c3)cc2)cc1]

150 mL of xylene was added to Intermediate 7-1 (10.0 g, 0.018 mol), 5′-bromo-1,1′:3′,1″-terphenyl (8.2 g, 0.027 mol), NaOtBu (5.1 g, 0.053 mol), Pd(dba)₂ (0.4 g, 0.7 mmol), and t-Bu₃P (0.3 g, 1.4 mmol), and the resulting mixture was stirred at 70° C. for 4 hours and reacted. After completion of the reaction, the resulting product was extracted and concentrated, and then subjected to column chromatography and recrystallization to obtain 10.7 g (yield 76.1%) of <Compound 7>.

LC/MS: m/z=791[(M)⁺]

### Synthesis Example 3: Synthesis of Compound 20

### (1) Preparation Example 1: Synthesis of Compound 20

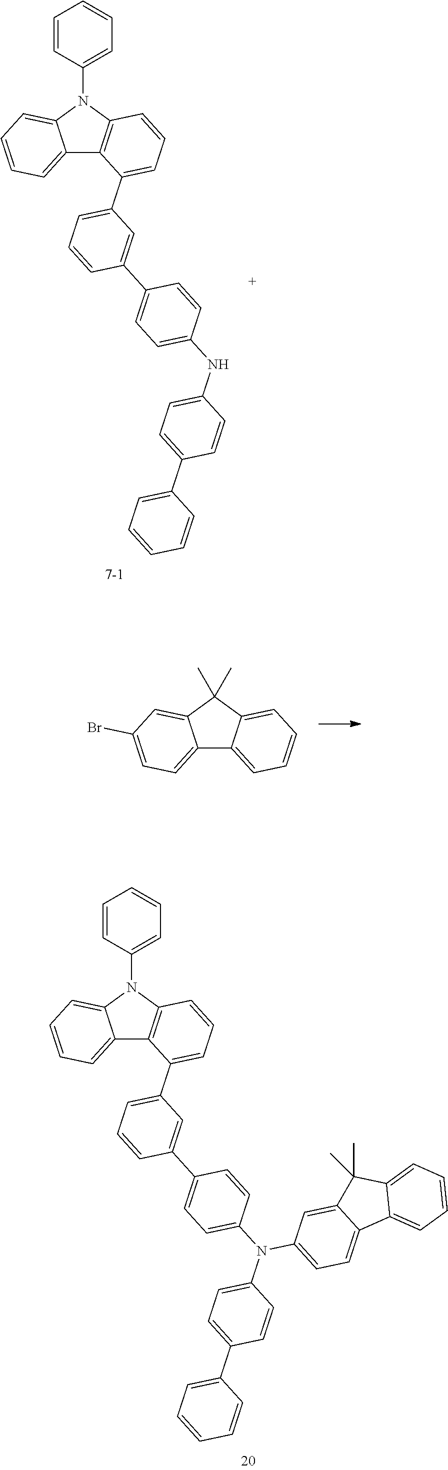 [SMILES: c1ccc(-c2ccc(Nc3ccc(-c4cccc(-c5cccc6c5c5ccccc5n6-c5ccccc5)c4)cc3)cc2)cc1 ; CC1(C)c2ccccc2-c2ccc(Br)cc21 ; CC1(C)c2ccccc2-c2ccc(N(c3ccc(-c4ccccc4)cc3)c3ccc(-c4cccc(-c5cccc6c5c5ccccc5n6-c5ccccc5)c4)cc3)cc21]

150 mL of xylene was added to Intermediate 7-1 (10.0 g, 0.018 mol), 2′-bromo-9,9-dimethylfluorene (7.3 g, 0.027 mol), NaOtBu (5.1 g, 0.053 mol), Pd(dba)₂ (0.4 g, 0.7 mmol), and t-Bu₃P (0.3 g, 1.4 mmol), and the resulting mixture was stirred at 70° C. for 4 hours and reacted. After completion of the reaction, the resulting product was extracted and concentrated, and then subjected to column chromatography and recrystallization to obtain 10.3 g (yield 76.8%) of <Compound 20>.

LC/MS: m/z=754[(M)⁺]

### Synthesis Example 4: Synthesis of Compound 32

### (1) Preparation Example 1: Synthesis of Compound 32

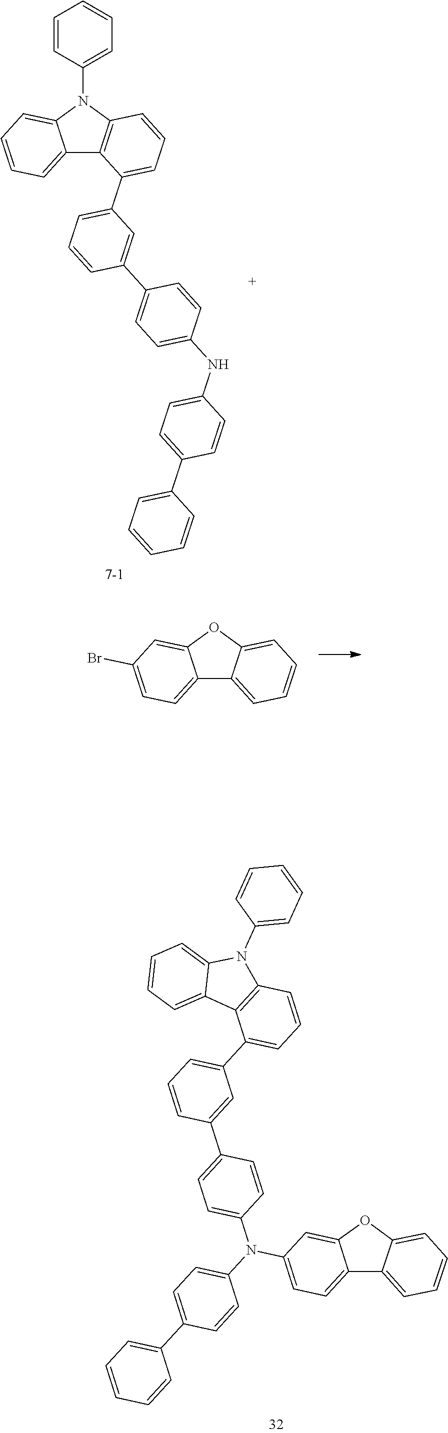 [SMILES: c1ccc(-c2ccc(Nc3ccc(-c4cccc(-c5cccc6c5c5ccccc5n6-c5ccccc5)c4)cc3)cc2)cc1 ; Brc1ccc2c(c1)oc1ccccc12 ; c1ccc(-c2ccc(N(c3ccc(-c4cccc(-c5cccc6c5c5ccccc5n6-c5ccccc5)c4)cc3)c3ccc4c(c3)oc3ccccc34)cc2)cc1]

150 mL of xylene was added to Intermediate 7-1 (10.0 g, 0.018 mol), 3-bromodibenzo[b,d]furan (6.6 g, 0.027 mol), NaOtBu (5.1 g, 0.053 mol), Pd(dba)₂ (0.4 g, 0.7 mmol), and t-Bu₃P (0.3 g, 1.4 mmol), and the resulting mixture was stirred at 70° C. for 4 hours and reacted. After completion of the reaction, the resulting product was extracted and concentrated, and then subjected to column chromatography and recrystallization to obtain 9.1 g (yield 70.3%) of <Compound 32>.

LC/MS: m/z=728[(M)⁺]

### Synthesis Example 5: Synthesis of Compound 53

### (1) Preparation Example 1: Synthesis of Intermediate 53-1

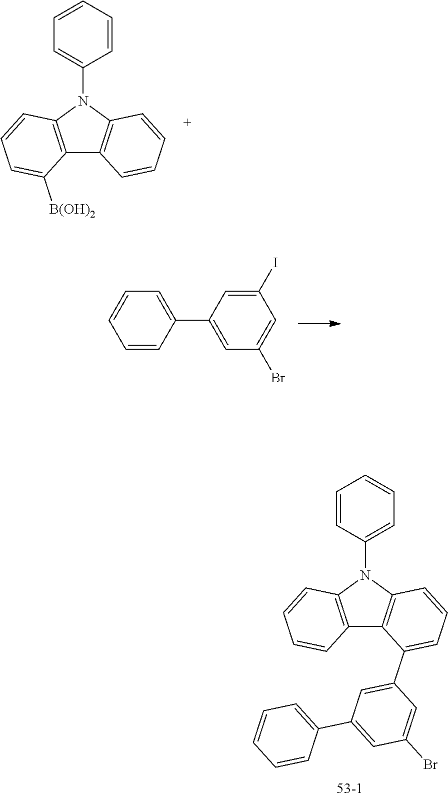 [SMILES: OB(O)c1cccc2c1c1ccccc1n2-c1ccccc1 ; Brc1cc(I)cc(-c2ccccc2)c1 ; Brc1cc(-c2ccccc2)cc(-c2cccc3c2c2ccccc2n3-c2ccccc2)c1]

200 mL of toluene, 50 mL of ethanol, and 50 mL of H₂O were added to 9-phenyl-9H-carbazol-4-yl-4-boronic acid (10.0 g, 0.035 mol), 3-bromo-5-iodo-1,1′-biphenyl (15.0 g, 0.042 mol), K₂CO₃ (14.4 g, 0.105 mol), and Pd(PPh₃)₄ (0.8 g, 0.0007 mol), and the resulting mixture was stirred at 80° C. for 6 hours and reacted. After completion of the reaction, the resulting product was extracted and concentrated, and then subjected to column chromatography to obtain 10.2 g (yield 61.7%) of <Intermediate 53-1>.

### (2) Preparation Example 2: Synthesis of Intermediate 53-2

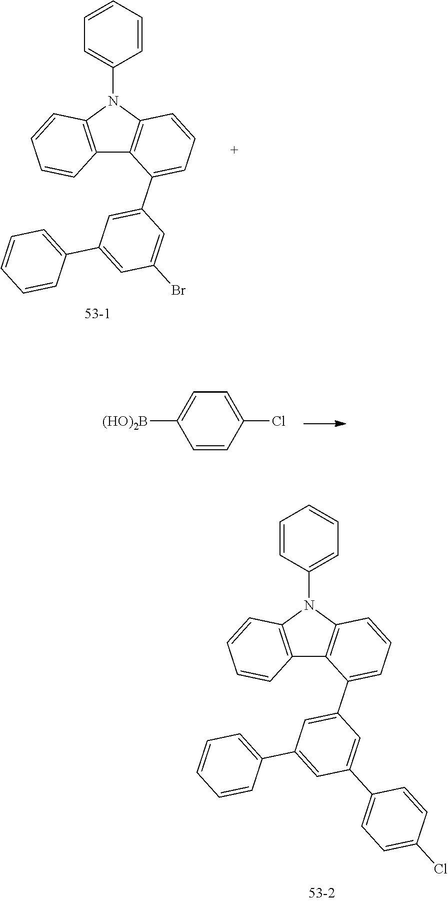 [SMILES: Brc1cc(-c2ccccc2)cc(-c2cccc3c2c2ccccc2n3-c2ccccc2)c1 ; OB(O)c1ccc(Cl)cc1 ; Clc1ccc(-c2cc(-c3ccccc3)cc(-c3cccc4c3c3ccccc3n4-c3ccccc3)c2)cc1]

200 mL of toluene, 50 mL of ethanol, and 50 mL of H₂O were added to Intermediate 53-1 (10.0 g, 0.021 mol), 4-chlorophenylboronic acid (4.0 g, 0.025 mol), K₂CO₃ (8.7 g, 0.063 mol), and Pd(PPh₃)₄ (0.5 g, 0.0004 mol), and the resulting mixture was stirred at 80° C. for 6 hours and reacted. After completion of the reaction, the resulting product was extracted and concentrated, and then subjected to column chromatography and recrystallization to obtain 8.6 g (yield 80.6%) of <Intermediate 53-2>.

### (3) Preparation Example 3. Synthesis of Compound 53

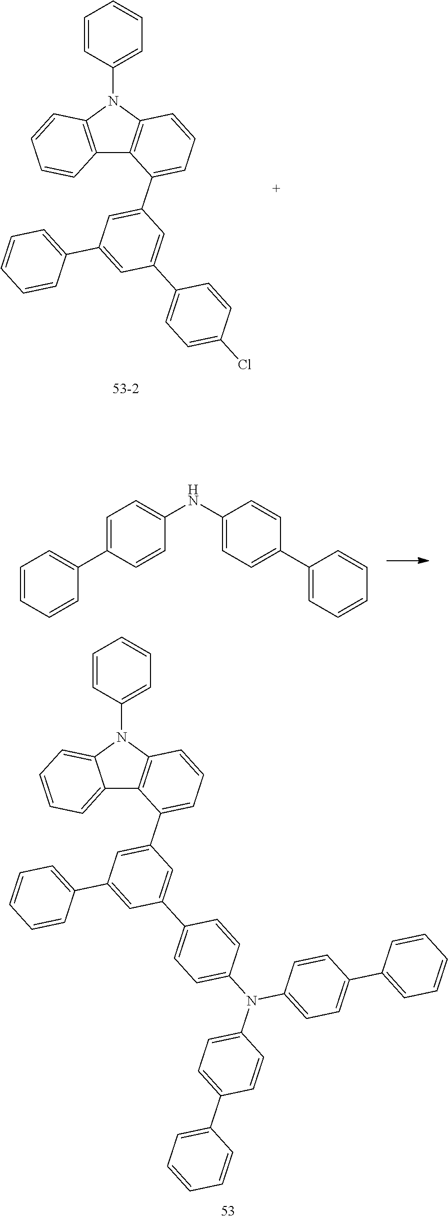 [SMILES: Clc1ccc(-c2cc(-c3ccccc3)cc(-c3cccc4c3c3ccccc3n4-c3ccccc3)c2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cc(-c5ccccc5)cc(-c5cccc6c5c5ccccc5n6-c5ccccc5)c4)cc3)cc2)cc1]

150 mL of xylene was added to Intermediate 53-2 (10.0 g, 0.020 mol), bis(4-biphenylyl)amine (9.5 g, 0.030 mol), NaOtBu (5.7 g, 0.059 mol), Pd(dba)₂ (0.5 g, 0.8 mmol), and t-BU₃P (0.3 g, 1.6 mmol), and the resulting mixture was stirred at 120° C. for 4 hours and reacted. After completion of the reaction, the resulting product was extracted and concentrated, and then subjected to column chromatography and recrystallization to obtain 9.8 g (yield 62.7%) of <Compound 53>.

LC/MS: m/z=790[(M)⁺]

### Synthesis Example 6: Synthesis of Compound 69

### (1) Preparation Example 1: Synthesis of Compound 69

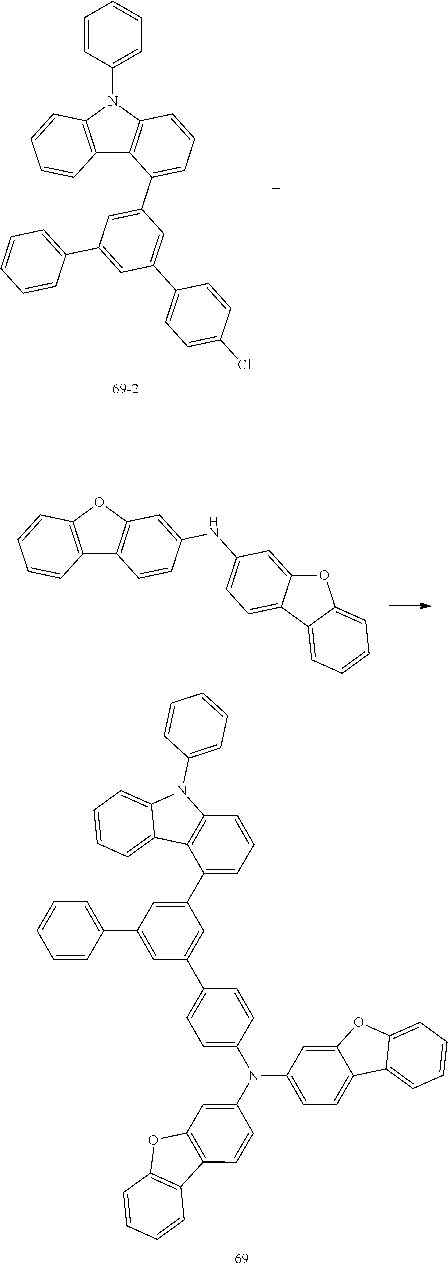 [SMILES: Clc1ccc(-c2cc(-c3ccccc3)cc(-c3cccc4c3c3ccccc3n4-c3ccccc3)c2)cc1 ; c1ccc2c(c1)oc1cc(Nc3ccc4c(c3)oc3ccccc34)ccc12 ; c1ccc(-c2cc(-c3ccc(N(c4ccc5c(c4)oc4ccccc45)c4ccc5c(c4)oc4ccccc45)cc3)cc(-c3cccc4c3c3ccccc3n4-c3ccccc3)c2)cc1]

150 mL of xylene was added to Intermediate 69-2 (10.0 g, 0.020 mol), bis(dibenzo[b,d]furan-3-yl)amine (10.4 g, 0.030 mol), NaOtBu (5.7 g, 0.059 mol), Pd(dba)₂ (0.5 g, 0.8 mmol), and t-BU₃P (0.3 g, 1.6 mmol), and the resulting mixture was stirred at 120° C. for 4 hours and reacted. After completion of the reaction, the resulting product was extracted and concentrated, and then subjected to column chromatography and recrystallization to obtain 10.5 g (yield 64.9%) of <Compound 69>.

LC/MS: m/z=818[(M)⁺]

### Synthesis Example 7: Synthesis of Compound 109

### (1) Preparation Example 1: Synthesis of Intermediate 109-1

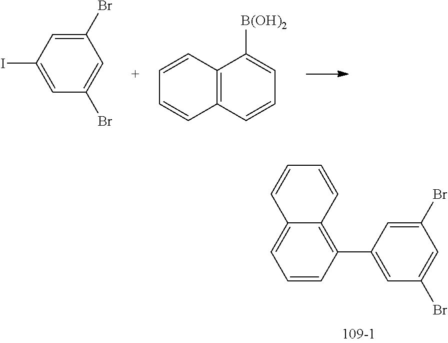 [SMILES: Brc1cc(Br)cc(I)c1 ; OB(O)c1cccc2ccccc12 ; Brc1cc(Br)cc(-c2cccc3ccccc23)c1]

200 mL of toluene, 50 mL of ethanol, and 50 mL of H₂O were added to 1,3-dibromo-5-chlorobenzene (10.0 g, 0.028 mol), naphthalene-1-boronic acid (5.7 g, 0.033 mol), K₂CO₃ (11.5 g, 0.083 mol), and Pd(PPh₃)₄ (0.6 g, 0.0006 mol), and the resulting mixture was stirred at 80° C. for 6 hours and reacted. After completion of the reaction, the resulting product was extracted and concentrated, and then subjected to column chromatography to obtain 5.3 g (yield 53.0%) of <Intermediate 109-1>.

### (2) Preparation Example 2: Synthesis of Intermediate 109-2

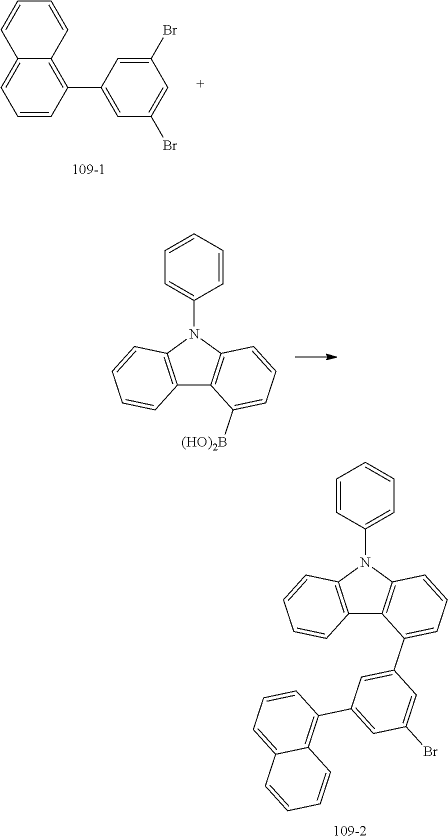 [SMILES: Brc1cc(Br)cc(-c2cccc3ccccc23)c1 ; OB(O)c1cccc2c1c1ccccc1n2-c1ccccc1 ; Brc1cc(-c2cccc3ccccc23)cc(-c2cccc3c2c2ccccc2n3-c2ccccc2)c1]

200 mL of toluene, 50 mL of ethanol, and 50 mL of H₂O were added to Intermediate 109-1 (10.0 g, 0.028 mol), 9-phenyl-9H-carbazol-4-yl-4-boronic acid (9.5 g, 0.033 mol), K₂CO₃ (11.5 g, 0.083 mol), and Pd(PPh₃)₄ (0.6 g, 0.0006 mol), and the resulting mixture was stirred at 80° C. for 6 hours and reacted. After completion of the reaction, the resulting product was extracted and concentrated, and then subjected to column chromatography to obtain 8.5 g (yield 58.7%) of <Intermediate 109-2>.

### (3) Preparation Example 3: Synthesis of Intermediate 109-3

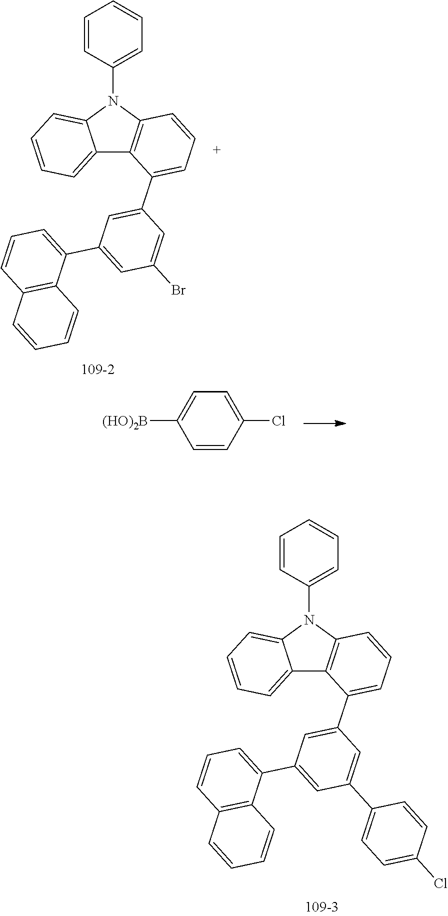 [SMILES: Brc1cc(-c2cccc3ccccc23)cc(-c2cccc3c2c2ccccc2n3-c2ccccc2)c1 ; OB(O)c1ccc(Cl)cc1 ; Clc1ccc(-c2cc(-c3cccc4ccccc34)cc(-c3cccc4c3c3ccccc3n4-c3ccccc3)c2)cc1]

200 mL of toluene, 50 mL of ethanol, and 50 mL of H₂O were added to Intermediate 109-2 (10.0 g, 0.019 mol), 4-chlorophenylboronic acid (3.6 g, 0.023 mol), K₂CO₃ (7.9 g, 0.057 mol), and Pd(PPh₃)₄ (0.4 g, 0.0004 mol), and the resulting mixture was stirred at 80° C. for 6 hours and reacted. After completion of the reaction, the resulting product was extracted and concentrated, and then subjected to column chromatography and recrystallization to obtain 7.78 g (yield 72.6%) of <Intermediate 109-3>.

### (4) Preparation Example 4: Synthesis of Compound 109

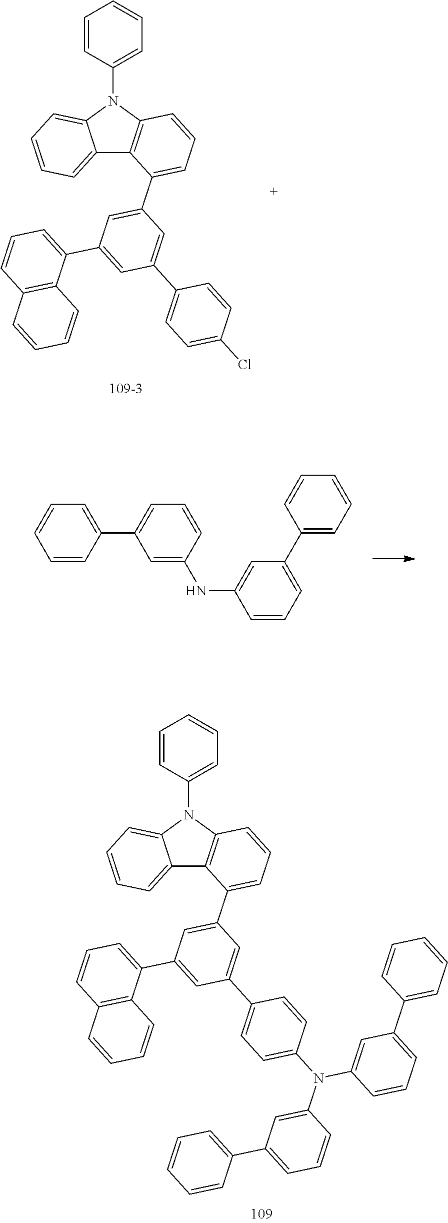 [SMILES: Clc1ccc(-c2cc(-c3cccc4ccccc34)cc(-c3cccc4c3c3ccccc3n4-c3ccccc3)c2)cc1 ; c1ccc(-c2cccc(Nc3cccc(-c4ccccc4)c3)c2)cc1 ; c1ccc(-c2cccc(N(c3ccc(-c4cc(-c5cccc6ccccc56)cc(-c5cccc6c5c5ccccc5n6-c5ccccc5)c4)cc3)c3cccc(-c4ccccc4)c3)c2)cc1]

150 mL of xylene was added to Intermediate 109-3 (10.0 g, 0.018 mol), bis(3-biphenylyl)amine (8.7 g, 0.027 mol), NaOtBu (5.2 g, 0.054 mol), Pd(dba)₂ (0.4 g, 0.7 mmol), and t-Bu₃P (0.3 g, 1.4 mmol), and the resulting mixture was stirred at 120° C. for 4 hours and reacted. After completion of the reaction, the resulting product was extracted and concentrated, and then subjected to column chromatography and recrystallization to obtain 9.8 g (yield 64.8%) of <Compound 109>.

LC/MS: m/z=840[(M)⁺]

### Synthesis Example 8: Synthesis of Compound 154

### (1) Preparation Example 1: Synthesis of Intermediate 154-1

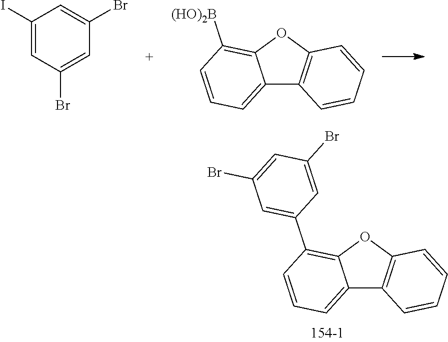

200 mL of toluene, 50 mL of ethanol, and 50 mL of H₂O were added to 1,3-dibromo-5-chlorobenzene (10.0 g, 0.028 mol), dibenzo[b,d]furan-4-ylboronic acid (7.03 g, 0.033 mol), K₂CO₃ (11.5 g, 0.083 mol), and Pd(PPh₃)₄ (0.6 g, 0.0006 mol), and the resulting mixture was stirred at 80° C. for 6 hours and reacted. After completion of the reaction, the resulting product was extracted and concentrated, and then subjected to column chromatography to obtain 10.5 g (yield 55.4%) of <Intermediate 154-1>.

### (2) Preparation Example 2: Synthesis of Intermediate 154-2

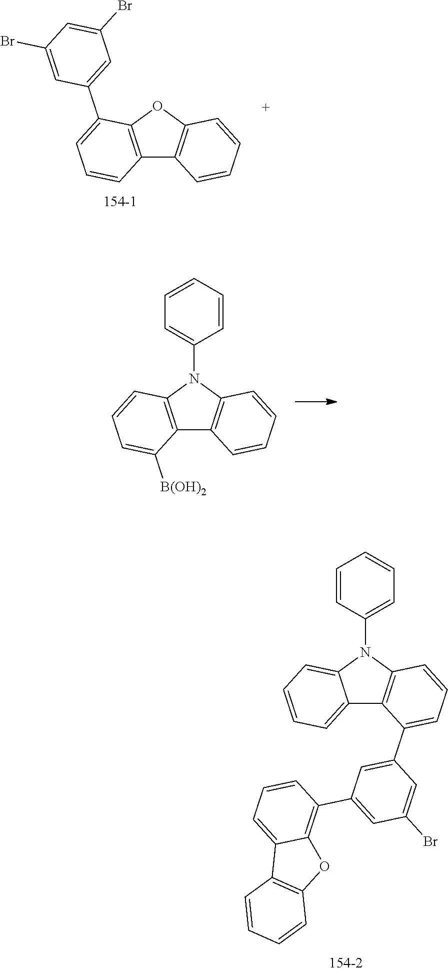 [SMILES: Brc1cc(Br)cc(-c2cccc3c2oc2ccccc23)c1 ; OB(O)c1cccc2c1c1ccccc1n2-c1ccccc1 ; Brc1cc(-c2cccc3c2oc2ccccc23)cc(-c2cccc3c2c2ccccc2n3-c2ccccc2)c1]

200 mL of toluene, 50 mL of ethanol, and 50 mL of H₂O were added to Intermediate 154-1 (10.0 g, 0.025 mol), 9-phenyl-9H-carbazol-4-yl-4-boronic acid (8.6 g, 0.030 mol), K₂CO₃ (10.3 g, 0.075 mol), and Pd(PPh₃)₄ (0.6 g, 0.0005 mol), and the resulting mixture was stirred at 80° C. for 6 hours and reacted. After completion of the reaction, the resulting product was extracted and concentrated, and then subjected to column chromatography to obtain 8.90 g (yield 63.4%) of <Intermediate 154-2>.

### (3) Preparation Example 3: Synthesis of Intermediate 154-3

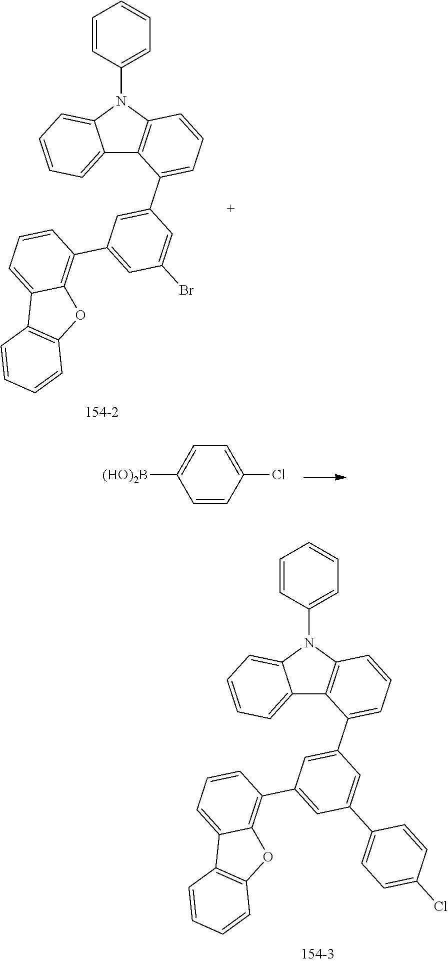 [SMILES: Brc1cc(-c2cccc3c2oc2ccccc23)cc(-c2cccc3c2c2ccccc2n3-c2ccccc2)c1 ; OB(O)c1ccc(Cl)cc1 ; Clc1ccc(-c2cc(-c3cccc4c3oc3ccccc34)cc(-c3cccc4c3c3ccccc3n4-c3ccccc3)c2)cc1]

200 mL of toluene, 50 mL of ethanol, and 50 mL of H₂O were added to Intermediate 154-2 (10.0 g, 0.018 mol), 4-chlorophenylboronic acid (3.3 g, 0.021 mol), K₂CO₃ (7.4 g, 0.053 mol), and Pd(PPh₃)₄ (0.4 g, 0.0004 mol), and the resulting mixture was stirred at 80° C. for 6 hours and reacted. After completion of the reaction, the resulting product was extracted and concentrated, and then subjected to column chromatography and recrystallization to obtain 8.7 g (yield 82.4%) of <Intermediate 154-3>.

### (4) Preparation Example 4: Synthesis of Compound 154

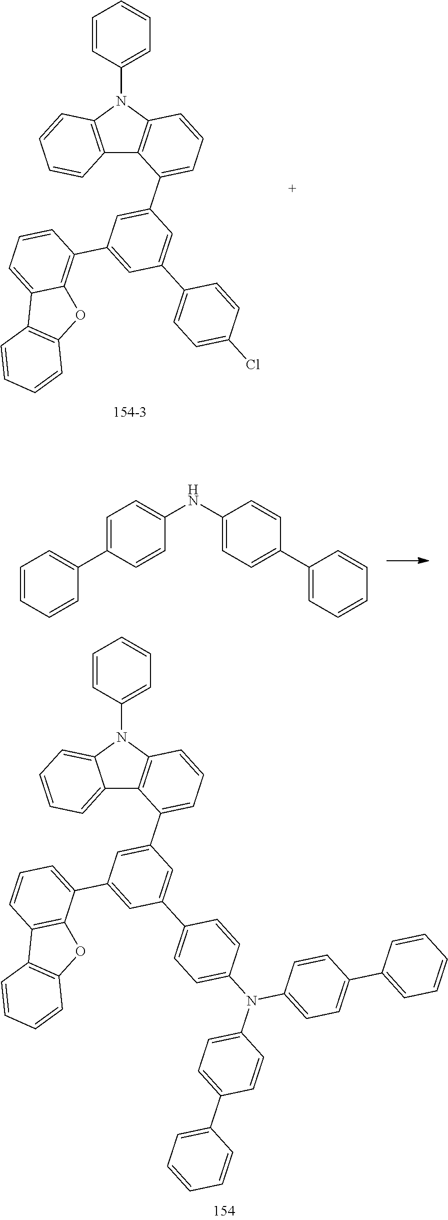 [SMILES: Clc1ccc(-c2cc(-c3cccc4c3oc3ccccc34)cc(-c3cccc4c3c3ccccc3n4-c3ccccc3)c2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cc(-c5cccc6c5oc5ccccc56)cc(-c5cccc6c5c5ccccc5n6-c5ccccc5)c4)cc3)cc2)cc1]

150 mL of xylene was added to Intermediate 154-3 (10.0 g, 0.017 mol), bis(4-biphenylyl)amine (8.1 g, 0.025 mol), NaOtBu (4.8 g, 0.050 mol), Pd(dba)₂ (0.4 g, 0.7 mmol), and t-BU₃P (0.3 g, 1.3 mmol), and the resulting mixture was stirred at 120° C. for 4 hours and reacted. After completion of the reaction, the resulting product was extracted and concentrated, and then subjected to column chromatography and recrystallization to obtain 10.7 g (yield 72.4%) of <Compound 154>.

LC/MS: m/z=880[(M)⁺]

### DEVICE EXAMPLES (EBL)

In exemplary embodiments according to the present invention, an ITO transparent electrode was patterned using an ITO glass substrate to which the ITO transparent electrode was attached on a glass substrate of 25 mm×25 mm×0.7 mm such that a light emitting area had a size of 2 mm×2 mm, and then washed. After the substrate was mounted in a vacuum chamber, a base pressure was set to 1×10⁻⁶ torr, and organic substances and a metal were deposited to have the following structure on the ITO.

### Device Examples 1 to 43

After an organic light emitting device having the following device structure was manufactured by employing a compound implemented by the present invention for an electron blocking layer, light emitting and driving characteristics of the compound implemented according to the present invention were measured.

ITO/hole injecting layer (HAT-CN, 5 nm)/hole transport layer (HT1, 100 nm)/electron blocking layer (10 nm)/light emitting layer (20 nm)/electron transport layer (ET1:Liq, 30 nm)/LiF (1 nm)/Al (100 nm)

After [HAT-CN]was film-formed to a thickness of 5 nm on top of an ITO transparent electrode to form a hole injecting layer, [HT1] was film-formed to a thickness of 100 nm to form a hole transport layer, the compound according to the present invention shown in the following Table 1 was film-formed to a thickness of 10 nm to form an electron blocking layer, and a light-emitting layer was formed by co-depositing [BH1] as a host compound and [BD1] as a dopant compound to a thickness of 20 nm. Thereafter, an electron transport layer (50% doping of the following [ET1] compound Liq) was deposited to a thickness of 30 nm, and then LiF was film-formed to a thickness of 1 nm to form an electron injecting layer, and Al was film-formed to a thickness of 100 nm, thereby manufacturing an organic light-emitting device.

### Device Comparative Example 1

An organic light emitting device for Device Comparative Example 1 was manufactured in the same manner as in the device structure in Examples 1 to 43, except that in the electron blocking layer, the following [EB1] was used instead of the compound according to the present invention.

### Device Comparative Example 2

An organic light emitting device for Device Comparative Example 2 was manufactured in the same manner as in the device structure in Examples 1 to 43, except that in the electron blocking layer, the following [EB2] was used instead of the compound according to the present invention.

### Device Comparative Example 3

An organic light emitting device for Device Comparative Example 3 was manufactured in the same manner as in the device structure in Examples 1 to 43, except that in the electron blocking layer, the following [EB3] was used instead of the compound according to the present invention.

### Device Comparative Example 4

An organic light emitting device for Device Comparative Example 4 was manufactured in the same manner as in the device structure in Examples 1 to 43, except that in the electron blocking layer, the following [EB4] was used instead of the compound according to the present invention.

### Experimental Example 1: Light-Emitting Characteristics of Device Examples 1 to 43

For the organic light-emitting devices manufactured by the Examples and the Comparative Examples, driving voltage, current efficiency and color coordinate were measured using a source meter (Model 237, Keithley) and a luminance meter (PR-650, Photo Research), and the result values based on 1,000 nits are shown in the following [Table 1].

TABLE 1

| | Electron | | | | |
|---|---|---|---|---|---|
| Example | blocking layer | V | cd/A | CIEx | CIEy |
| 1 | Formula 2 | 4.33 | 7.12 | 0.1344 | 0.1349 |
| 2 | Formula 4 | 4.46 | 7.09 | 0.1340 | 0.1362 |
| 3 | Formula 7 | 4.27 | 6.93 | 0.1324 | 0.1376 |
| 4 | Formula 11 | 4.05 | 7.28 | 0.1348 | 0.1351 |
| 5 | Formula 20 | 4.16 | 7.07 | 0.1328 | 0.1375 |
| 6 | Formula 24 | 4.44 | 6.83 | 0.1337 | 0.1362 |
| 7 | Formula 25 | 4.48 | 6.89 | 0.1354 | 0.1346 |
| 8 | Formula 26 | 4.29 | 7.30 | 0.1342 | 0.1358 |
| 9 | Formula 32 | 4.31 | 7.31 | 0.1338 | 0.1362 |
| 10 | Formula 34 | 4.42 | 6.96 | 0.1352 | 0.1349 |
| 11 | Formula 41 | 4.46 | 7.09 | 0.1340 | 0.1362 |
| 12 | Formula 43 | 4.46 | 7.09 | 0.1340 | 0.1362 |
| 13 | Formula 48 | 4.05 | 7.28 | 0.1348 | 0.1351 |
| 14 | Formula 53 | 4.42 | 7.19 | 0.1314 | 0.1384 |
| 15 | Formula 55 | 4.05 | 7.31 | 0.1343 | 0.1357 |
| 16 | Formula 60 | 4.37 | 6.85 | 0.1352 | 0.1348 |
| 17 | Formula 64 | 4.44 | 6.79 | 0.1326 | 0.1371 |
| 18 | Formula 69 | 4.31 | 7.07 | 0.1351 | 0.1349 |
| 19 | Formula 73 | 4.42 | 7.19 | 0.1314 | 0.1384 |
| 20 | Formula 76 | 4.42 | 7.19 | 0.1314 | 0.1384 |
| 21 | Formula 80 | 4.29 | 6.85 | 0.1328 | 0.1375 |
| 22 | Formula 83 | 4.37 | 6.96 | 0.1321 | 0.1361 |
| 23 | Formula 85 | 4.23 | 7.11 | 0.1359 | 0.1342 |
| 24 | Formula 91 | 4.20 | 6.88 | 0.1334 | 0.1366 |
| 25 | Formula 97 | 4.35 | 7.03 | 0.1346 | 0.1354 |
| 26 | Formula 99 | 4.13 | 7.28 | 0.1333 | 0.1367 |
| 27 | Formula 104 | 4.23 | 7.11 | 0.1359 | 0.1342 |
| 28 | Formula 109 | 4.39 | 7.12 | 0.1344 | 0.1356 |
| 29 | Formula 116 | 4.54 | 7.26 | 0.1359 | 0.1341 |
| 30 | Formula 121 | 4.42 | 7.55 | 0.1354 | 0.1348 |
| 31 | Formula 122 | 4.30 | 6.92 | 0.1325 | 0.1375 |
| 32 | Formula 125 | 4.19 | 7.07 | 0.1354 | 0.1346 |
| 33 | Formula 128 | 4.42 | 6.89 | 0.1338 | 0.1364 |
| 34 | Formula 131 | 4.33 | 7.03 | 0.1350 | 0.1352 |
| 35 | Formula 132 | 4.19 | 7.07 | 0.1354 | 0.1346 |
| 36 | Formula 144 | 4.38 | 6.94 | 0.1319 | 0.1381 |
| 37 | Formula 148 | 4.29 | 7.01 | 0.1315 | 0.1385 |
| 38 | Formula 154 | 4.40 | 6.82 | 0.1324 | 0.1372 |
| 39 | Formula 155 | 4.23 | 7.06 | 0.1322 | 0.1378 |
| 40 | Formula 161 | 4.05 | 7.22 | 0.1311 | 0.1386 |
| 41 | Formula 165 | 4.23 | 6.84 | 0.1304 | 0.1392 |
| 42 | Formula 167 | 4.35 | 6.99 | 0.1331 | 0.1369 |
| 43 | Formula 169 | 4.17 | 6.83 | 0.1329 | 0.1371 |
| Comparative | EB1 | 4.67 | 6.65 | 0.1353 | 0.1517 |
| Example 1 | | | | | |
| Comparative | EB2 | 4.57 | 6.62 | 0.1310 | 0.1390 |
| Example 2 | | | | | |
| Comparative | EB3 | 4.72 | 6.44 | 0.1324 | 0.1378 |
| Example 3 | | | | | |
| Comparative | EB4 | 4.65 | 6.48 | 0.1342 | 0.1358 |
| Example 4 | | | | | |

Referring to the results shown in [Table 1] above, it can be confirmed that when the compound according to the present invention is employed to an electron blocking layer in the organic light-emitting device, low-voltage driving characteristics and luminescent characteristics such as luminous efficiency and quantum efficiency are significantly excellent compared to the devices (Comparative Examples 1 to 4) in which compounds used as conventional materials for an electron blocking layer, which are contrasted with the characteristic structures of the compounds according to the present invention, were employed.

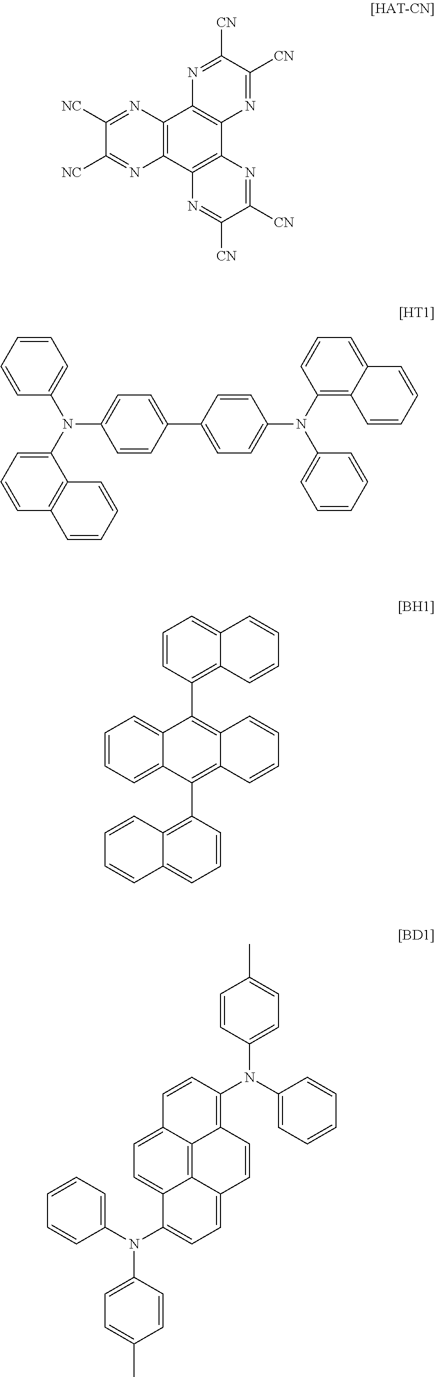

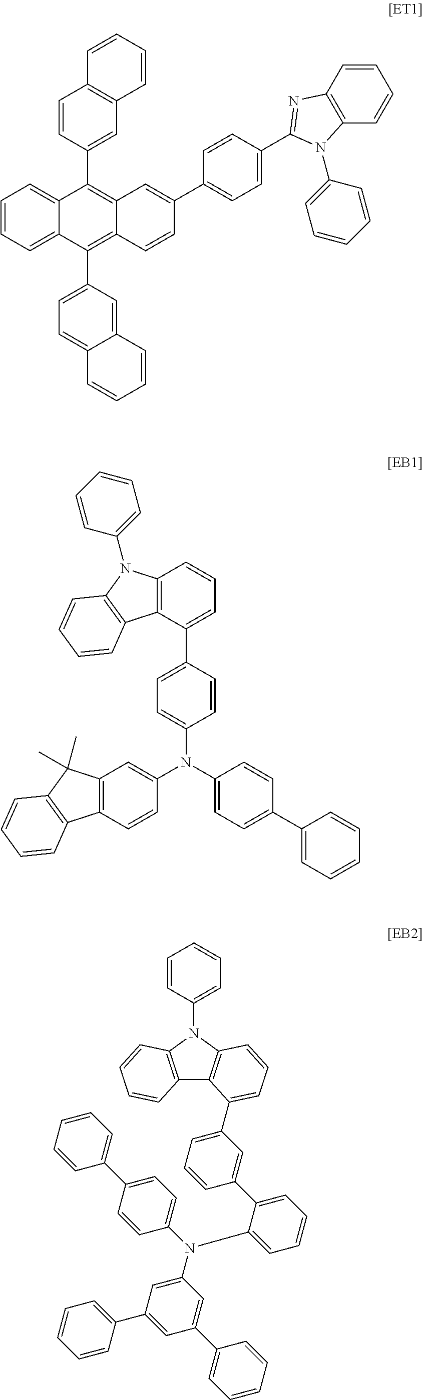 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC1(C)c2ccccc2-c2ccc(N(c3ccc(-c4ccccc4)cc3)c3ccc(-c4cccc5c4c4ccccc4n5-c4ccccc4)cc3)cc21 ; c1ccc(-c2ccc(N(c3cc(-c4ccccc4)cc(-c4ccccc4)c3)c3ccccc3-c3cccc(-c4cccc5c4c4ccccc4n5-c4ccccc4)c3)cc2)cc1]

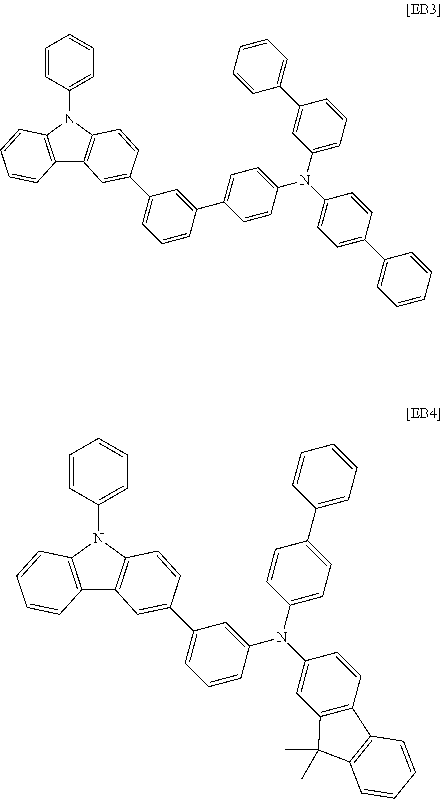 [SMILES: c1ccc(-c2ccc(N(c3ccc(-c4cccc(-c5ccc6c(c5)c5ccccc5n6-c5ccccc5)c4)cc3)c3cccc(-c4ccccc4)c3)cc2)cc1 ; CC1(C)c2ccccc2-c2ccc(N(c3ccc(-c4ccccc4)cc3)c3cccc(-c4ccc5c(c4)c4ccccc4n5-c4ccccc4)c3)cc21]

### INDUSTRIAL APPLICABILITY

Since the organic compound according to the present invention has excellent hole injection and transport ability, excellent triplet exciton confinement ability, excellent electron blocking ability, and excellent stability in a thin film state, an organic light-emitting device using the organic compound in an organic layer such as an electron blocking layer or a hole transport layer has significantly excellent device characteristics such as low-voltage driving, long service life characteristics, and luminous efficiency compared to conventional devices, and can be industrially usefully used in various lighting devices and display devices.

1. An organic compound represented by the following Formula I: 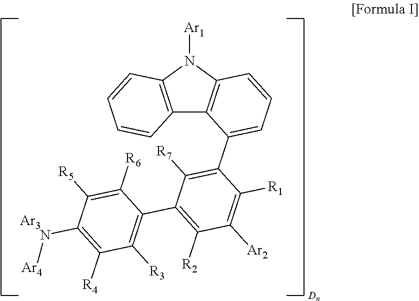
wherein, in [Formula I] above,
Ar₁ is any one selected from a substituted or unsubstituted aryl group having 6 to 30 carbon atoms and a substituted or unsubstituted heteroaryl group having 2 to 30 carbon atoms,
Ar₂ is any one selected from hydrogen, deuterium, a substituted or unsubstituted aryl group having 6 to 30 carbon atoms, or a substituted or unsubstituted heteroaryl group having 2 to 30 carbon atoms (provided that a carbazole group is excluded),
R₁ to R₇ are the same as or different from each other, and are each independently hydrogen or deuterium,
Ar₃ and Ar₄ are the same as or different from each other, and are each independently any one selected from a substituted or unsubstituted aryl group having 6 to 30 carbon atoms and a substituted or unsubstituted heteroaryl group having 2 to 30 carbon atoms, and
D is deuterium, n means the number of hydrogen atoms in [Formula I] above, which are replaced with deuterium atoms (D), and n is an integer from 0 to 60.

2. The organic compound of claim 1, wherein in the definitions of Ar₁ to Ar₄, substituted or unsubstituted means that each of Ar₁ to Ar₄ is substituted with one or two or more substituents selected from deuterium, a cyano group, a halogen group, a hydroxyl group, a nitro group, an alkyl group, a halogenated alkyl group, a deuterated alkyl group, an alkoxy group, a halogenated alkoxy group, a deuterated alkoxy group, a cycloalkyl group, a heterocycloalkyl group, an aryl group, a heteroaryl group, and a silyl group, is substituted with a substituent to which two or more substituents among the above substituents are linked, or has no substituent.

3. The organic compound of claim 1, wherein [Formula I] above is a compound in which hydrogen present in [Formula I] is partially substituted with deuterium (D), and the deuterium (D) substitution rate is 10 to 90%.

4. The organic compound of claim 3, wherein the deuterium (D) substitution rate is 20 to 80%.

5. The organic compound of claim 3, wherein the deuterium (D) substitution rate is 30 to 70%.

6. The organic compound of claim 1, wherein [Formula I] above is any one selected from among [Formula 1] to [Formula 169] below:

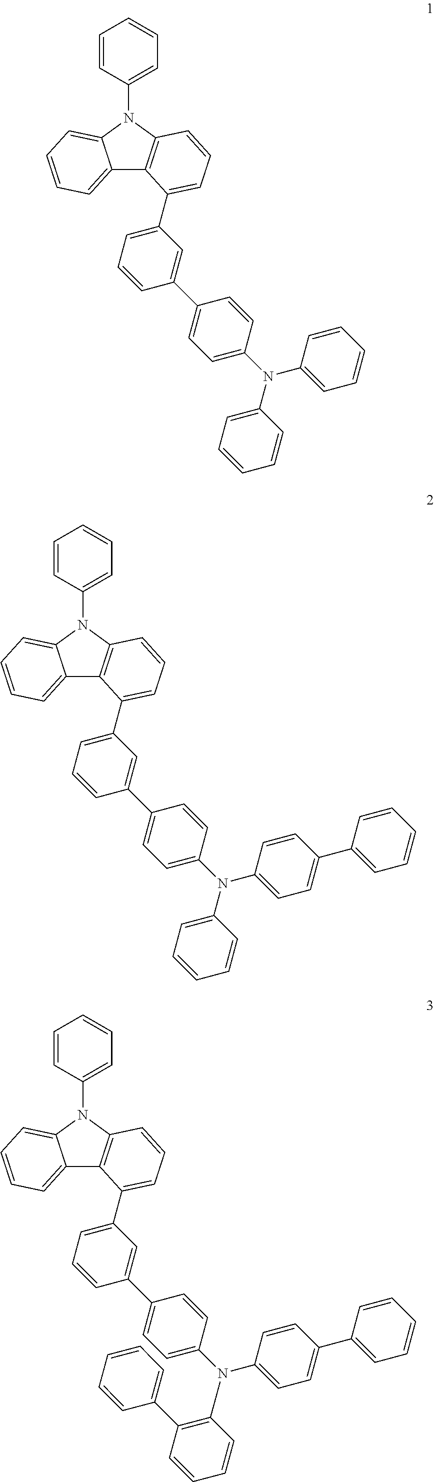 [SMILES: c1ccc(N(c2ccccc2)c2ccc(-c3cccc(-c4cccc5c4c4ccccc4n5-c4ccccc4)c3)cc2)cc1 ; c1ccc(-c2ccc(N(c3ccccc3)c3ccc(-c4cccc(-c5cccc6c5c5ccccc5n6-c5ccccc5)c4)cc3)cc2)cc1 ; c1ccc(-c2ccc(N(c3ccc(-c4cccc(-c5cccc6c5c5ccccc5n6-c5ccccc5)c4)cc3)c3ccccc3-c3ccccc3)cc2)cc1]

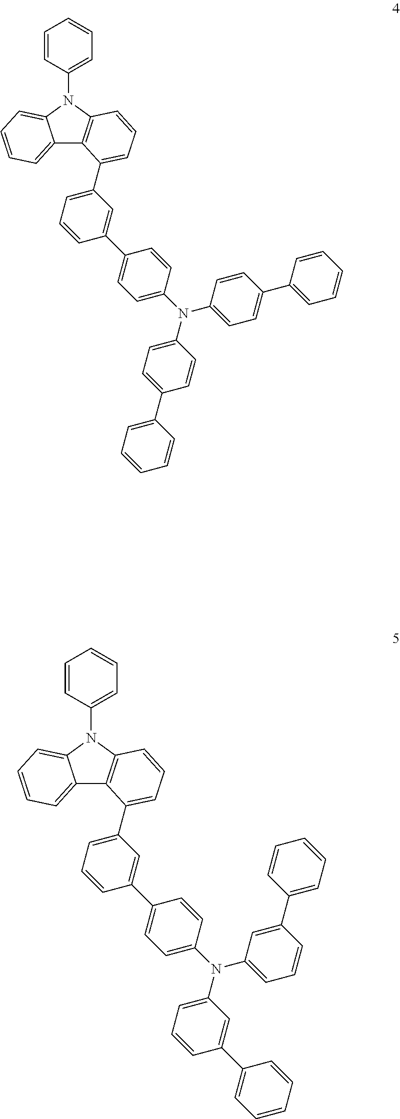 [SMILES: c1ccc(-c2ccc(N(c3ccc(-c4ccccc4)cc3)c3ccc(-c4cccc(-c5cccc6c5c5ccccc5n6-c5ccccc5)c4)cc3)cc2)cc1 ; c1ccc(-c2cccc(N(c3ccc(-c4cccc(-c5cccc6c5c5ccccc5n6-c5ccccc5)c4)cc3)c3cccc(-c4ccccc4)c3)c2)cc1]

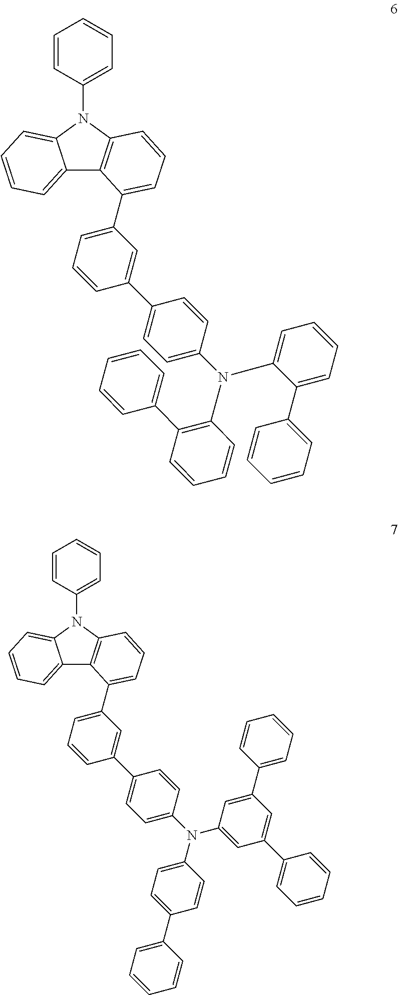 [SMILES: c1ccc(-c2ccccc2N(c2ccc(-c3cccc(-c4cccc5c4c4ccccc4n5-c4ccccc4)c3)cc2)c2ccccc2-c2ccccc2)cc1 ; c1ccc(-c2ccc(N(c3ccc(-c4cccc(-c5cccc6c5c5ccccc5n6-c5ccccc5)c4)cc3)c3cc(-c4ccccc4)cc(-c4ccccc4)c3)cc2)cc1]

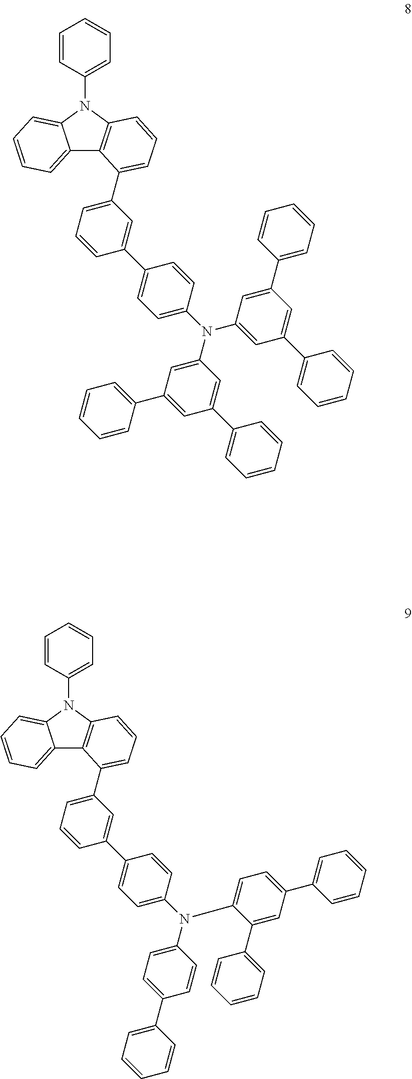 [SMILES: c1ccc(-c2cc(-c3ccccc3)cc(N(c3ccc(-c4cccc(-c5cccc6c5c5ccccc5n6-c5ccccc5)c4)cc3)c3cc(-c4ccccc4)cc(-c4ccccc4)c3)c2)cc1 ; c1ccc(-c2ccc(N(c3ccc(-c4cccc(-c5cccc6c5c5ccccc5n6-c5ccccc5)c4)cc3)c3ccc(-c4ccccc4)cc3-c3ccccc3)cc2)cc1]

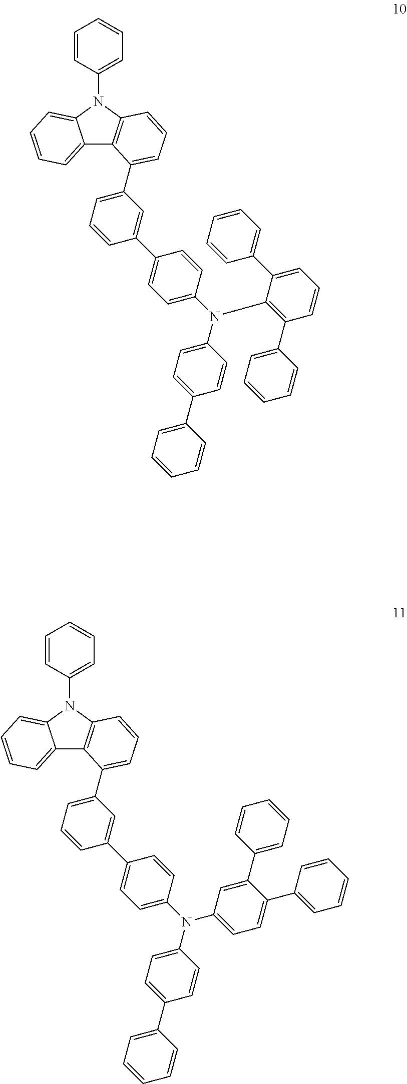 [SMILES: c1ccc(-c2ccc(N(c3ccc(-c4cccc(-c5cccc6c5c5ccccc5n6-c5ccccc5)c4)cc3)c3c(-c4ccccc4)cccc3-c3ccccc3)cc2)cc1 ; c1ccc(-c2ccc(N(c3ccc(-c4cccc(-c5cccc6c5c5ccccc5n6-c5ccccc5)c4)cc3)c3ccc(-c4ccccc4)c(-c4ccccc4)c3)cc2)cc1]

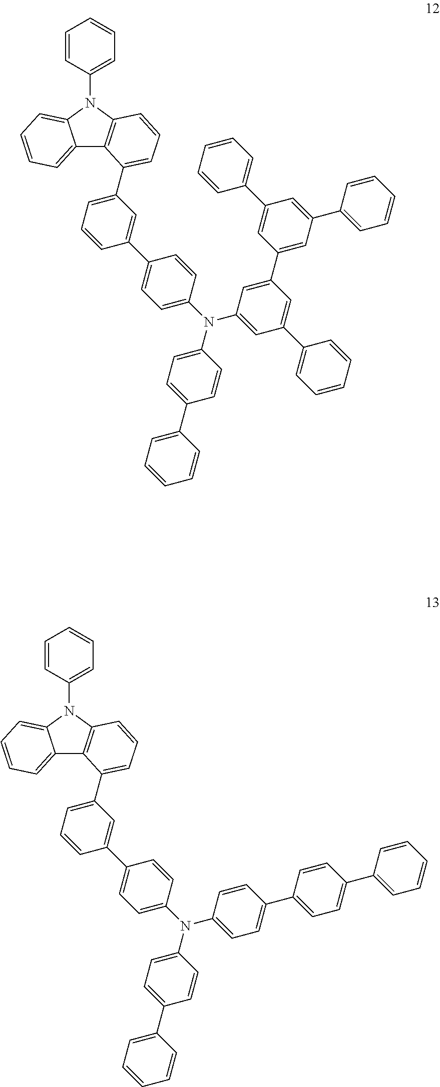 [SMILES: c1ccc(-c2ccc(N(c3ccc(-c4cccc(-c5cccc6c5c5ccccc5n6-c5ccccc5)c4)cc3)c3cc(-c4ccccc4)cc(-c4cc(-c5ccccc5)cc(-c5ccccc5)c4)c3)cc2)cc1 ; c1ccc(-c2ccc(-c3ccc(N(c4ccc(-c5ccccc5)cc4)c4ccc(-c5cccc(-c6cccc7c6c6ccccc6n7-c6ccccc6)c5)cc4)cc3)cc2)cc1]

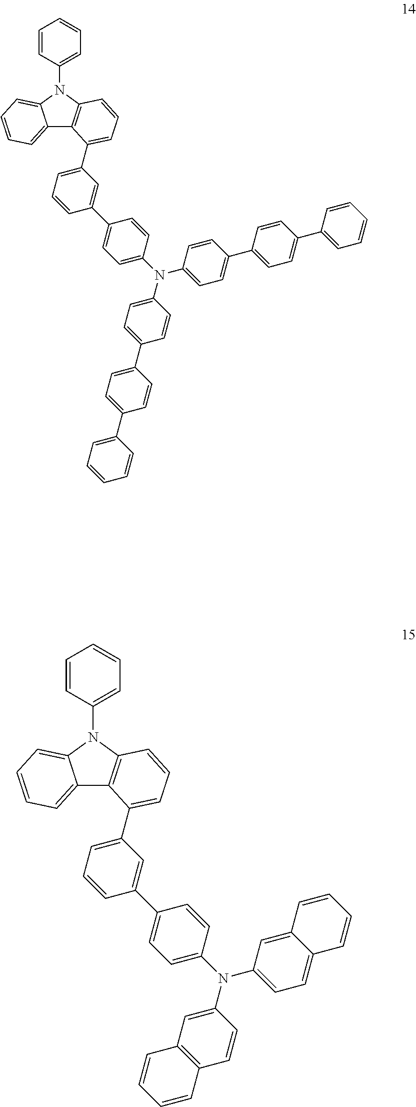 [SMILES: c1ccc(-c2ccc(-c3ccc(N(c4ccc(-c5ccc(-c6ccccc6)cc5)cc4)c4ccc(-c5cccc(-c6cccc7c6c6ccccc6n7-c6ccccc6)c5)cc4)cc3)cc2)cc1 ; c1ccc(-n2c3ccccc3c3c(-c4cccc(-c5ccc(N(c6ccc7ccccc7c6)c6ccc7ccccc7c6)cc5)c4)cccc32)cc1]

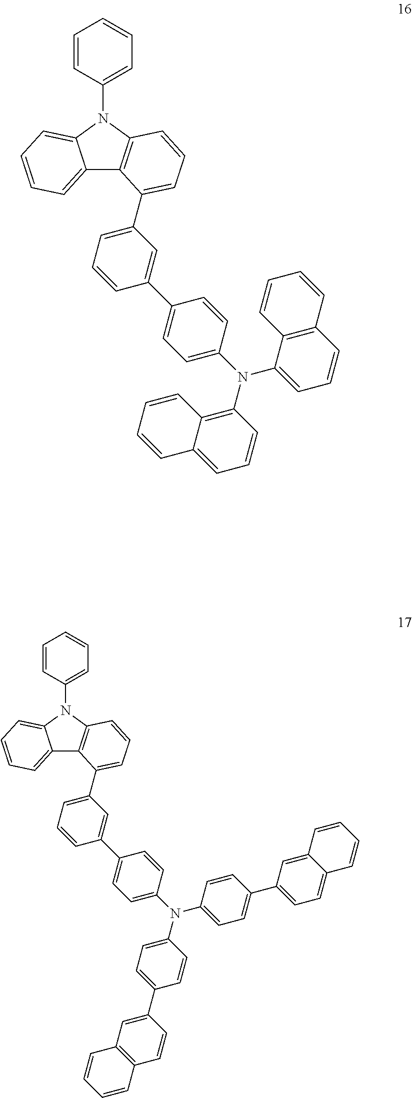 [SMILES: c1ccc(-n2c3ccccc3c3c(-c4cccc(-c5ccc(N(c6cccc7ccccc67)c6cccc7ccccc67)cc5)c4)cccc32)cc1 ; c1ccc(-n2c3ccccc3c3c(-c4cccc(-c5ccc(N(c6ccc(-c7ccc8ccccc8c7)cc6)c6ccc(-c7ccc8ccccc8c7)cc6)cc5)c4)cccc32)cc1]

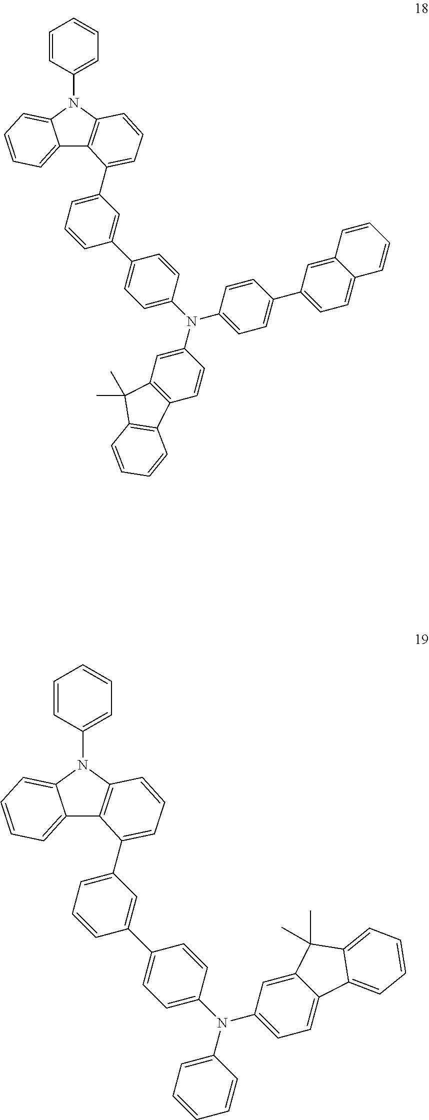 [SMILES: CC1(C)c2ccccc2-c2ccc(N(c3ccc(-c4cccc(-c5cccc6c5c5ccccc5n6-c5ccccc5)c4)cc3)c3ccc(-c4ccc5ccccc5c4)cc3)cc21 ; CC1(C)c2ccccc2-c2ccc(N(c3ccccc3)c3ccc(-c4cccc(-c5cccc6c5c5ccccc5n6-c5ccccc5)c4)cc3)cc21]

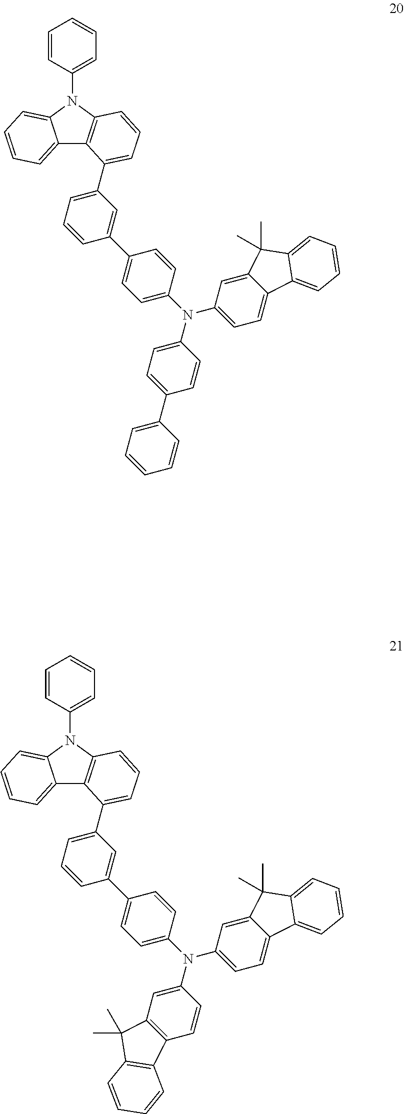 [SMILES: CC1(C)c2ccccc2-c2ccc(N(c3ccc(-c4ccccc4)cc3)c3ccc(-c4cccc(-c5cccc6c5c5ccccc5n6-c5ccccc5)c4)cc3)cc21 ; CC1(C)c2ccccc2-c2ccc(N(c3ccc(-c4cccc(-c5cccc6c5c5ccccc5n6-c5ccccc5)c4)cc3)c3ccc4c(c3)C(C)(C)c3ccccc3-4)cc21]

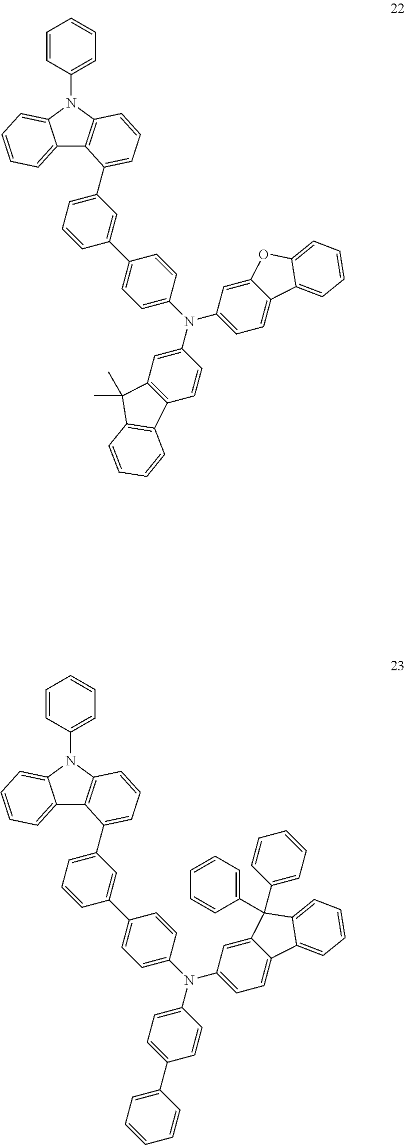 [SMILES: CC1(C)c2ccccc2-c2ccc(N(c3ccc(-c4cccc(-c5cccc6c5c5ccccc5n6-c5ccccc5)c4)cc3)c3ccc4c(c3)oc3ccccc34)cc21 ; c1ccc(-c2ccc(N(c3ccc(-c4cccc(-c5cccc6c5c5ccccc5n6-c5ccccc5)c4)cc3)c3ccc4c(c3)C(c3ccccc3)(c3ccccc3)c3ccccc3-4)cc2)cc1]

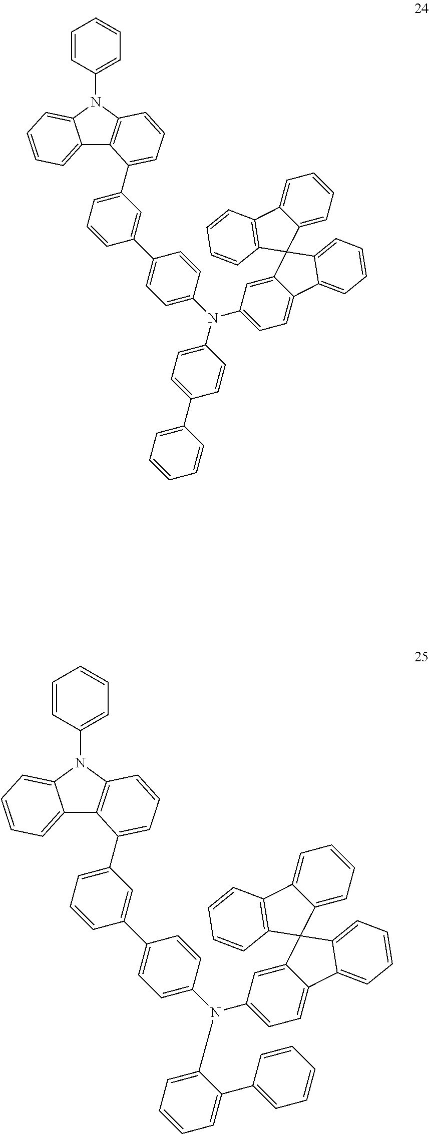 [SMILES: c1ccc(-c2ccc(N(c3ccc(-c4cccc(-c5cccc6c5c5ccccc5n6-c5ccccc5)c4)cc3)c3ccc4c(c3)C3(c5ccccc5-c5ccccc53)c3ccccc3-4)cc2)cc1 ; c1ccc(-c2ccccc2N(c2ccc(-c3cccc(-c4cccc5c4c4ccccc4n5-c4ccccc4)c3)cc2)c2ccc3c(c2)C2(c4ccccc4-c4ccccc42)c2ccccc2-3)cc1]

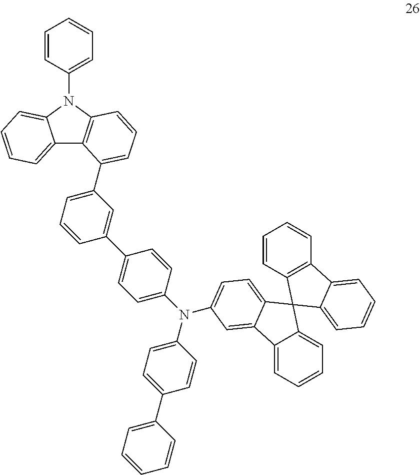 [SMILES: c1ccc(-c2ccc(N(c3ccc(-c4cccc(-c5cccc6c5c5ccccc5n6-c5ccccc5)c4)cc3)c3ccc4c(c3)-c3ccccc3C43c4ccccc4-c4ccccc43)cc2)cc1]

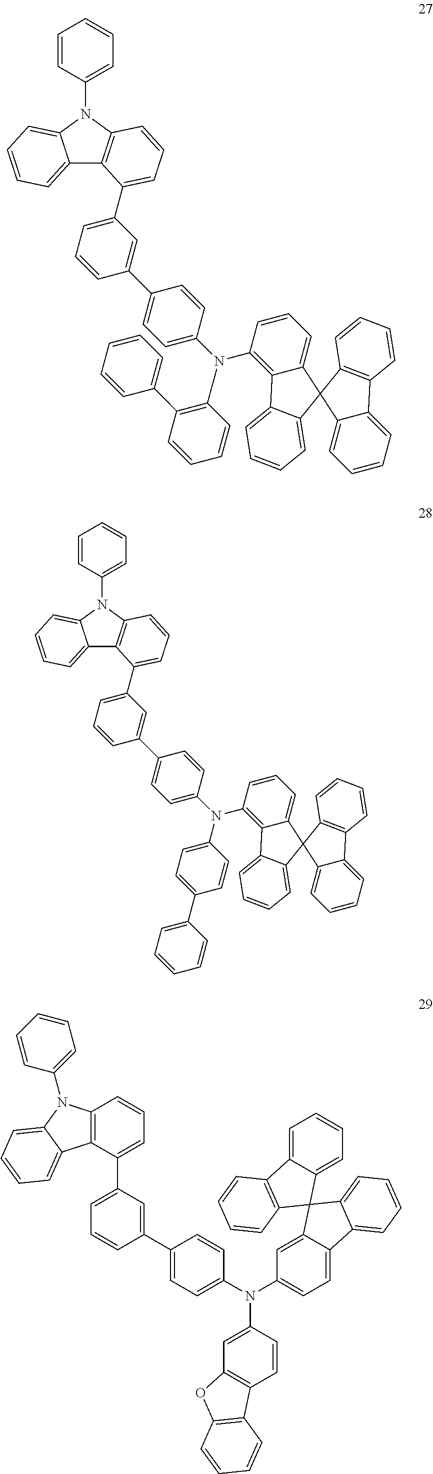 [SMILES: c1ccc(-c2ccccc2N(c2ccc(-c3cccc(-c4cccc5c4c4ccccc4n5-c4ccccc4)c3)cc2)c2cccc3c2-c2ccccc2C32c3ccccc3-c3ccccc32)cc1 ; c1ccc(-c2ccc(N(c3ccc(-c4cccc(-c5cccc6c5c5ccccc5n6-c5ccccc5)c4)cc3)c3cccc4c3-c3ccccc3C43c4ccccc4-c4ccccc43)cc2)cc1 ; c1ccc(-n2c3ccccc3c3c(-c4cccc(-c5ccc(N(c6ccc7c(c6)C6(c8ccccc8-c8ccccc86)c6ccccc6-7)c6ccc7c(c6)oc6ccccc67)cc5)c4)cccc32)cc1]

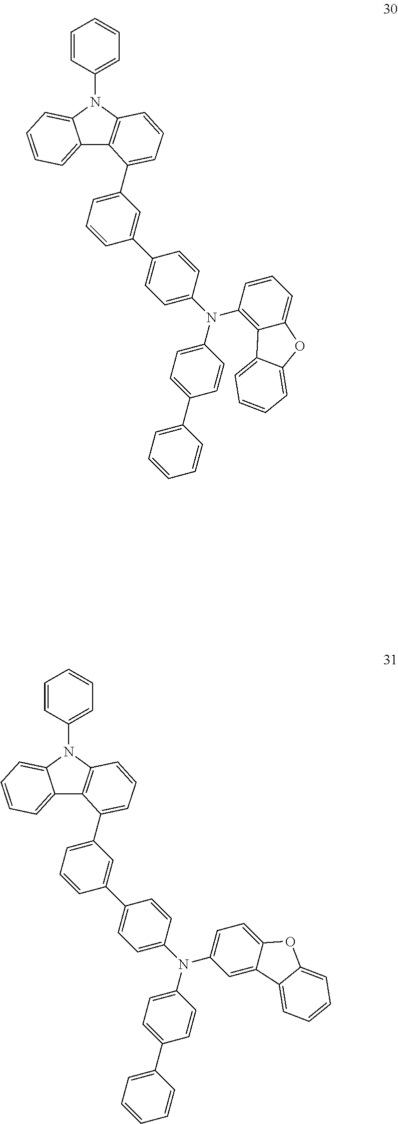 [SMILES: c1ccc(-c2ccc(N(c3ccc(-c4cccc(-c5cccc6c5c5ccccc5n6-c5ccccc5)c4)cc3)c3cccc4oc5ccccc5c34)cc2)cc1 ; c1ccc(-c2ccc(N(c3ccc(-c4cccc(-c5cccc6c5c5ccccc5n6-c5ccccc5)c4)cc3)c3ccc4oc5ccccc5c4c3)cc2)cc1]

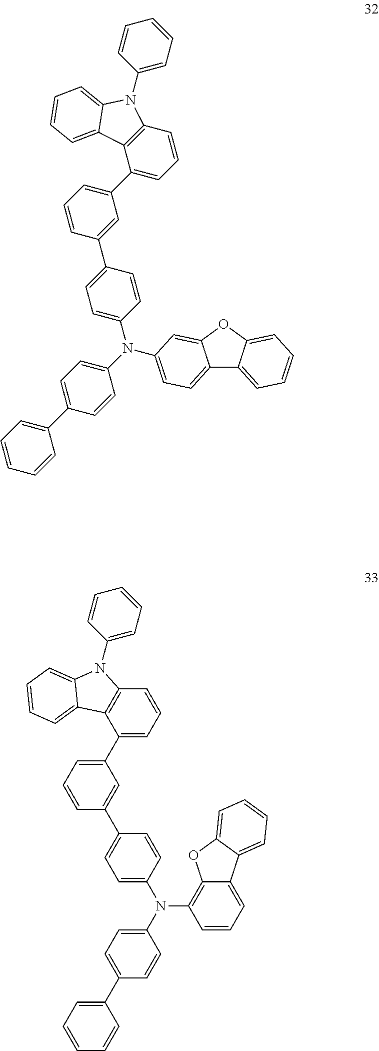 [SMILES: c1ccc(-c2ccc(N(c3ccc(-c4cccc(-c5cccc6c5c5ccccc5n6-c5ccccc5)c4)cc3)c3ccc4c(c3)oc3ccccc34)cc2)cc1 ; c1ccc(-c2ccc(N(c3ccc(-c4cccc(-c5cccc6c5c5ccccc5n6-c5ccccc5)c4)cc3)c3cccc4c3oc3ccccc34)cc2)cc1]

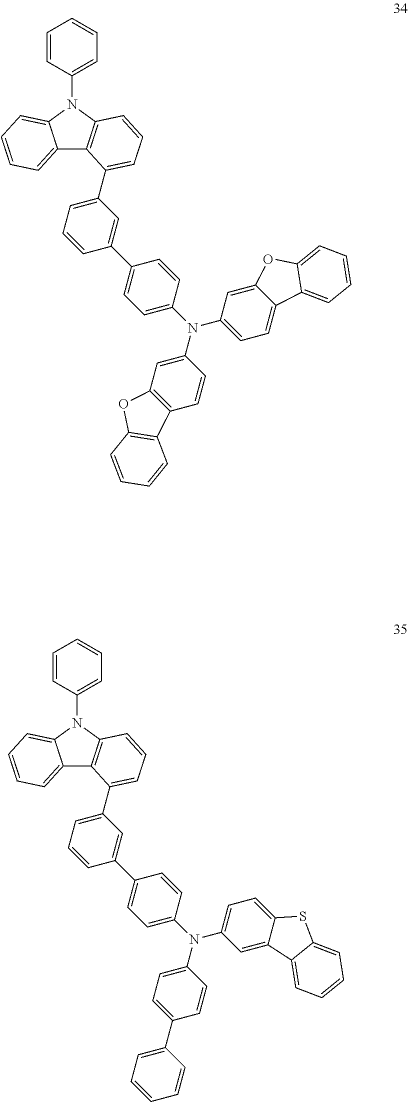 [SMILES: c1ccc(-n2c3ccccc3c3c(-c4cccc(-c5ccc(N(c6ccc7c(c6)oc6ccccc67)c6ccc7c(c6)oc6ccccc67)cc5)c4)cccc32)cc1 ; c1ccc(-c2ccc(N(c3ccc(-c4cccc(-c5cccc6c5c5ccccc5n6-c5ccccc5)c4)cc3)c3ccc4sc5ccccc5c4c3)cc2)cc1]

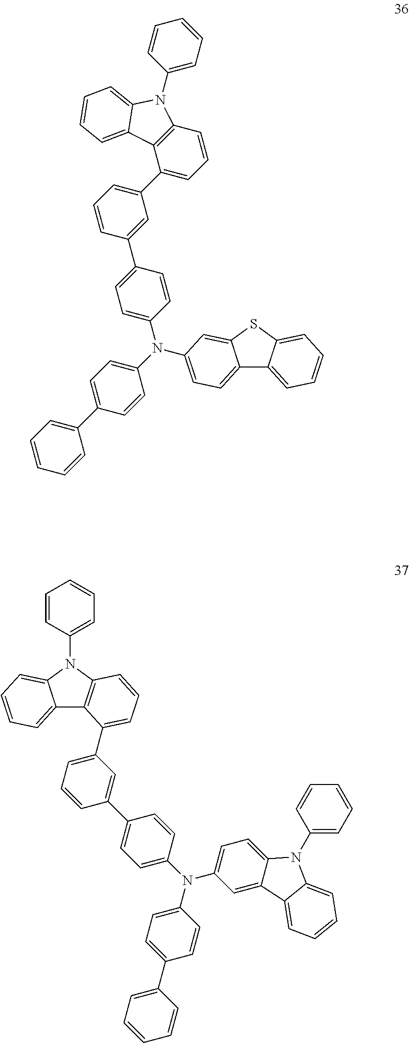 [SMILES: c1ccc(-c2ccc(N(c3ccc(-c4cccc(-c5cccc6c5c5ccccc5n6-c5ccccc5)c4)cc3)c3ccc4c(c3)sc3ccccc34)cc2)cc1 ; c1ccc(-c2ccc(N(c3ccc(-c4cccc(-c5cccc6c5c5ccccc5n6-c5ccccc5)c4)cc3)c3ccc4c(c3)c3ccccc3n4-c3ccccc3)cc2)cc1]

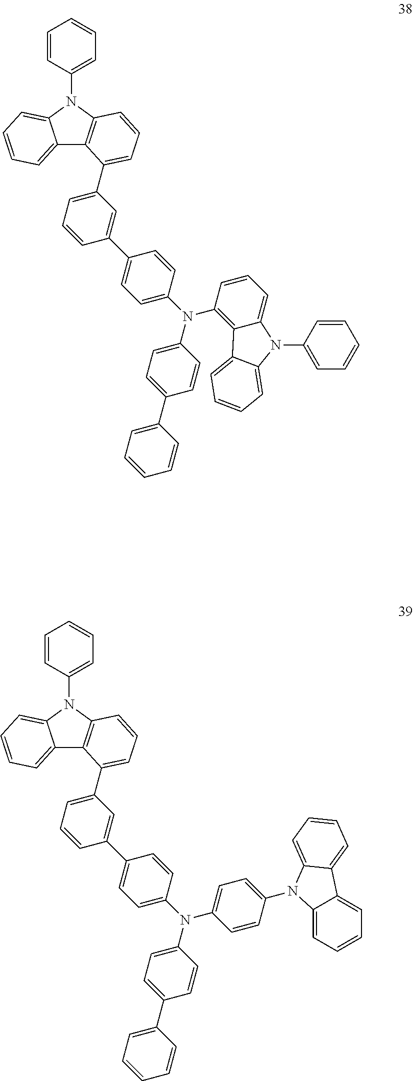 [SMILES: c1ccc(-c2ccc(N(c3ccc(-c4cccc(-c5cccc6c5c5ccccc5n6-c5ccccc5)c4)cc3)c3cccc4c3c3ccccc3n4-c3ccccc3)cc2)cc1 ; c1ccc(-c2ccc(N(c3ccc(-c4cccc(-c5cccc6c5c5ccccc5n6-c5ccccc5)c4)cc3)c3ccc(-n4c5ccccc5c5ccccc54)cc3)cc2)cc1]

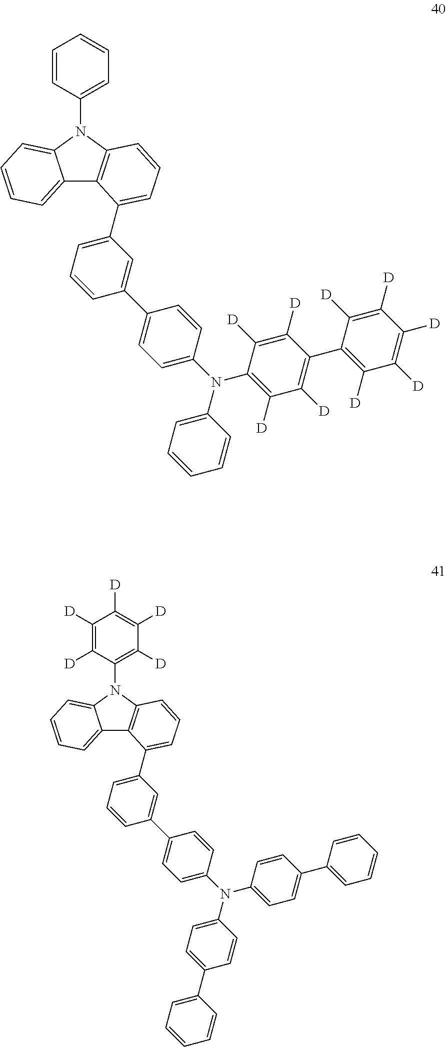 [SMILES: [2H]c1c([2H])c([2H])c(-c2c([2H])c([2H])c(N(c3ccccc3)c3ccc(-c4cccc(-c5cccc6c5c5ccccc5n6-c5ccccc5)c4)cc3)c([2H])c2[2H])c([2H])c1[2H] ; [2H]c1c([2H])c([2H])c(-n2c3ccccc3c3c(-c4cccc(-c5ccc(N(c6ccc(-c7ccccc7)cc6)c6ccc(-c7ccccc7)cc6)cc5)c4)cccc32)c([2H])c1[2H]]

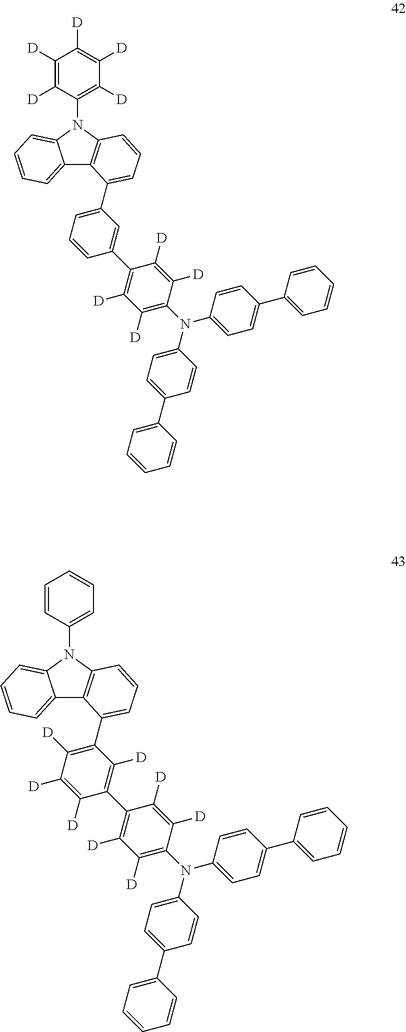 [SMILES: [2H]c1c([2H])c([2H])c(-n2c3ccccc3c3c(-c4cccc(-c5c([2H])c([2H])c(N(c6ccc(-c7ccccc7)cc6)c6ccc(-c7ccccc7)cc6)c([2H])c5[2H])c4)cccc32)c([2H])c1[2H] ; [2H]c1c([2H])c(-c2c([2H])c([2H])c(N(c3ccc(-c4ccccc4)cc3)c3ccc(-c4ccccc4)cc3)c([2H])c2[2H])c([2H])c(-c2cccc3c2c2ccccc2n3-c2ccccc2)c1[2H]]

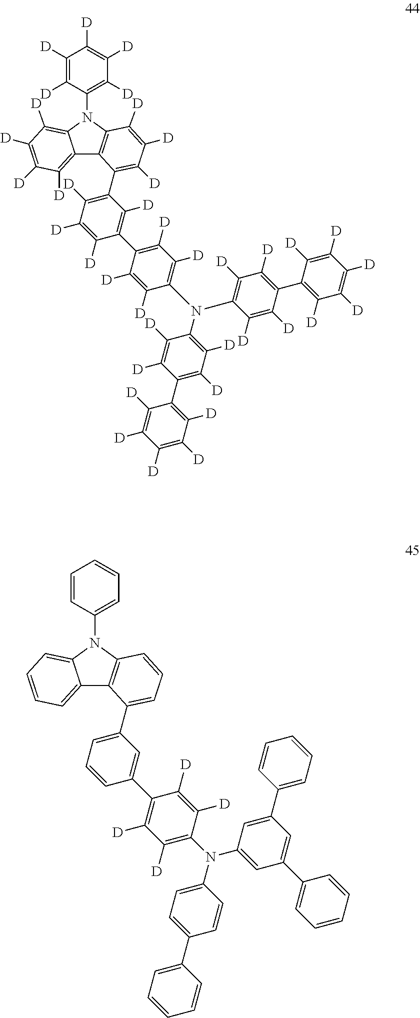 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c(-c5c([2H])c([2H])c([2H])c6c5c5c([2H])c([2H])c([2H])c([2H])c5n6-c5c([2H])c([2H])c([2H])c([2H])c5[2H])c4[2H])c([2H])c3[2H])c([2H])c2[2H])c([2H])c1[2H] ; [2H]c1c([2H])c(N(c2ccc(-c3ccccc3)cc2)c2cc(-c3ccccc3)cc(-c3ccccc3)c2)c([2H])c([2H])c1-c1cccc(-c2cccc3c2c2ccccc2n3-c2ccccc2)c1]

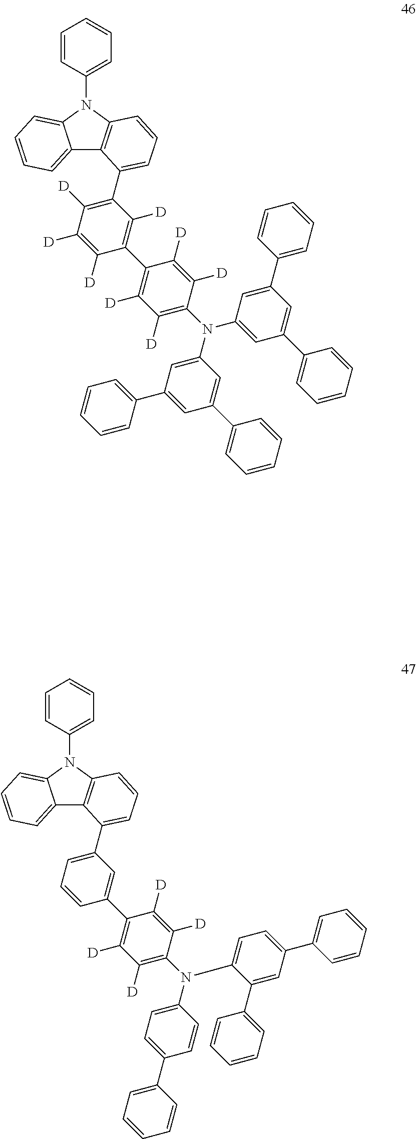 [SMILES: [2H]c1c([2H])c(-c2c([2H])c([2H])c(N(c3cc(-c4ccccc4)cc(-c4ccccc4)c3)c3cc(-c4ccccc4)cc(-c4ccccc4)c3)c([2H])c2[2H])c([2H])c(-c2cccc3c2c2ccccc2n3-c2ccccc2)c1[2H] ; [2H]c1c([2H])c(N(c2ccc(-c3ccccc3)cc2)c2ccc(-c3ccccc3)cc2-c2ccccc2)c([2H])c([2H])c1-c1cccc(-c2cccc3c2c2ccccc2n3-c2ccccc2)c1]

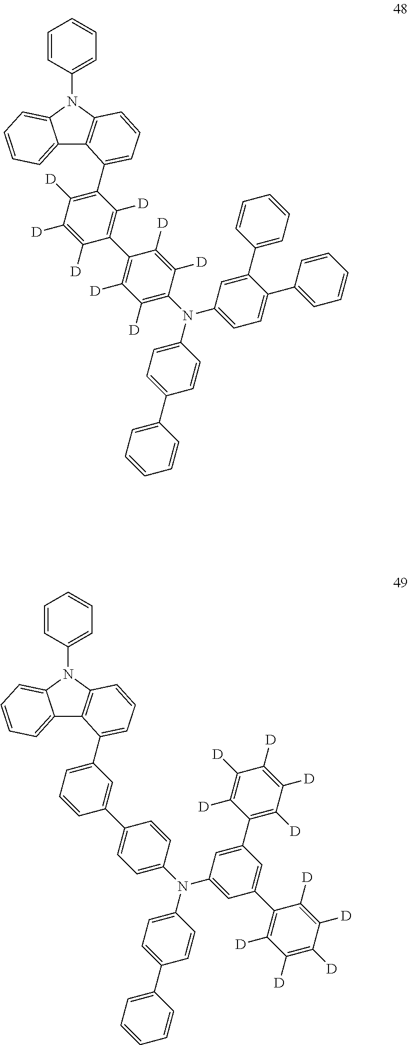 [SMILES: [2H]c1c([2H])c(-c2c([2H])c([2H])c(N(c3ccc(-c4ccccc4)cc3)c3ccc(-c4ccccc4)c(-c4ccccc4)c3)c([2H])c2[2H])c([2H])c(-c2cccc3c2c2ccccc2n3-c2ccccc2)c1[2H] ; [2H]c1c([2H])c([2H])c(-c2cc(-c3c([2H])c([2H])c([2H])c([2H])c3[2H])cc(N(c3ccc(-c4ccccc4)cc3)c3ccc(-c4cccc(-c5cccc6c5c5ccccc5n6-c5ccccc5)c4)cc3)c2)c([2H])c1[2H]]

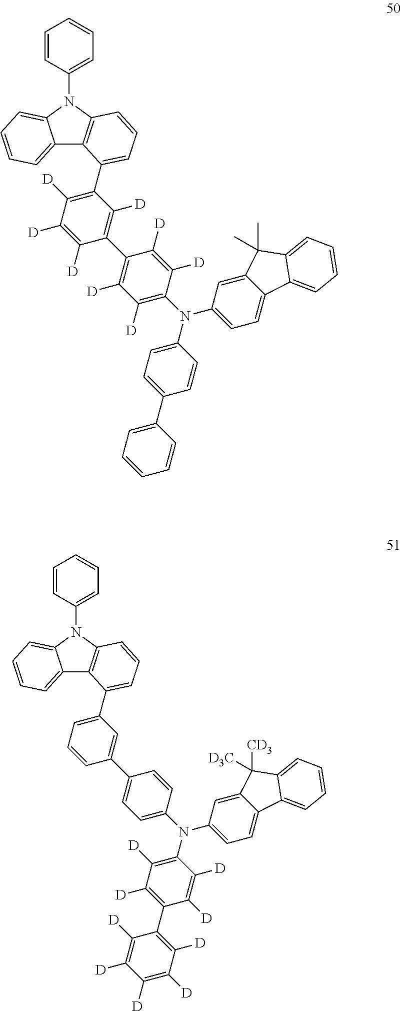 [SMILES: [2H]c1c([2H])c(-c2c([2H])c([2H])c(N(c3ccc(-c4ccccc4)cc3)c3ccc4c(c3)C(C)(C)c3ccccc3-4)c([2H])c2[2H])c([2H])c(-c2cccc3c2c2ccccc2n3-c2ccccc2)c1[2H] ; [2H]c1c([2H])c([2H])c(-c2c([2H])c([2H])c(N(c3ccc(-c4cccc(-c5cccc6c5c5ccccc5n6-c5ccccc5)c4)cc3)c3ccc4c(c3)C(C([2H])([2H])[2H])(C([2H])([2H])[2H])c3ccccc3-4)c([2H])c2[2H])c([2H])c1[2H]]

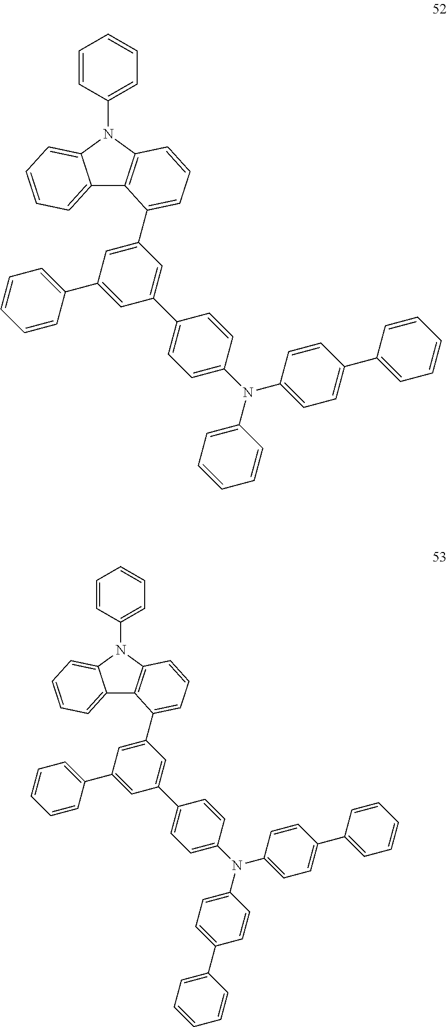 [SMILES: c1ccc(-c2ccc(N(c3ccccc3)c3ccc(-c4cc(-c5ccccc5)cc(-c5cccc6c5c5ccccc5n6-c5ccccc5)c4)cc3)cc2)cc1 ; c1ccc(-c2ccc(N(c3ccc(-c4ccccc4)cc3)c3ccc(-c4cc(-c5ccccc5)cc(-c5cccc6c5c5ccccc5n6-c5ccccc5)c4)cc3)cc2)cc1]

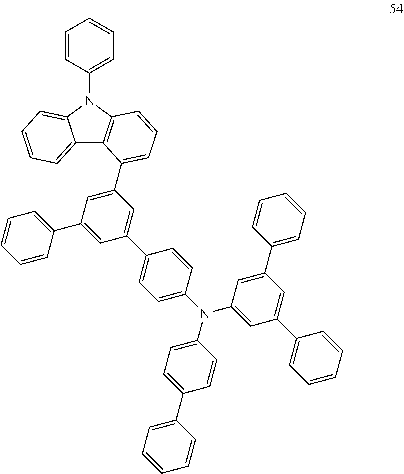 [SMILES: c1ccc(-c2ccc(N(c3ccc(-c4cc(-c5ccccc5)cc(-c5cccc6c5c5ccccc5n6-c5ccccc5)c4)cc3)c3cc(-c4ccccc4)cc(-c4ccccc4)c3)cc2)cc1]

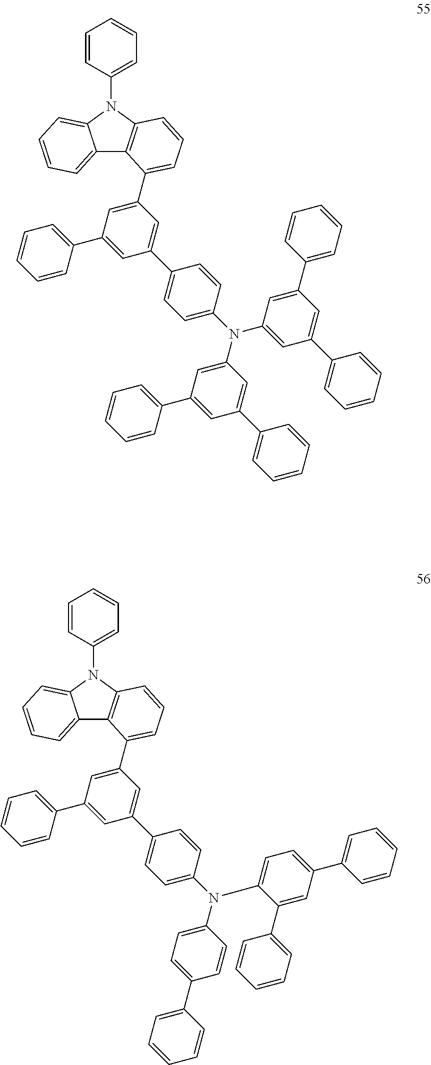 [SMILES: c1ccc(-c2cc(-c3ccc(N(c4cc(-c5ccccc5)cc(-c5ccccc5)c4)c4cc(-c5ccccc5)cc(-c5ccccc5)c4)cc3)cc(-c3cccc4c3c3ccccc3n4-c3ccccc3)c2)cc1 ; c1ccc(-c2ccc(N(c3ccc(-c4cc(-c5ccccc5)cc(-c5cccc6c5c5ccccc5n6-c5ccccc5)c4)cc3)c3ccc(-c4ccccc4)cc3-c3ccccc3)cc2)cc1]

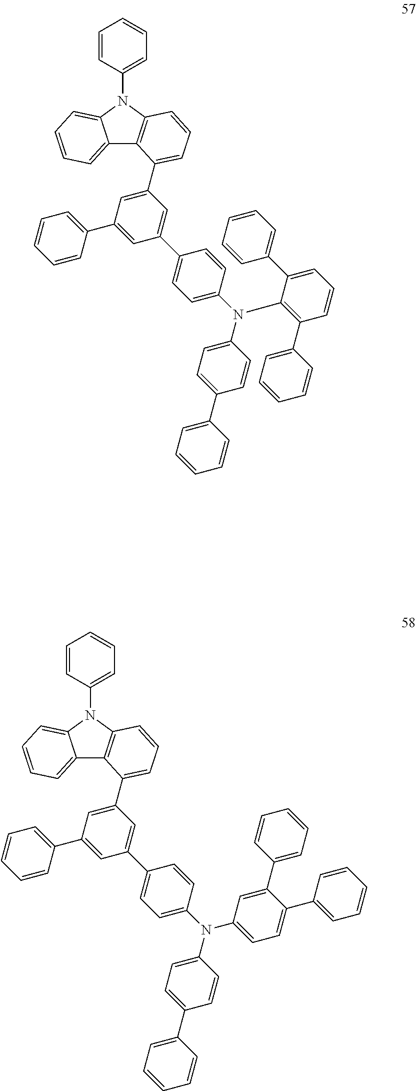 [SMILES: c1ccc(-c2ccc(N(c3ccc(-c4cc(-c5ccccc5)cc(-c5cccc6c5c5ccccc5n6-c5ccccc5)c4)cc3)c3c(-c4ccccc4)cccc3-c3ccccc3)cc2)cc1 ; c1ccc(-c2ccc(N(c3ccc(-c4cc(-c5ccccc5)cc(-c5cccc6c5c5ccccc5n6-c5ccccc5)c4)cc3)c3ccc(-c4ccccc4)c(-c4ccccc4)c3)cc2)cc1]

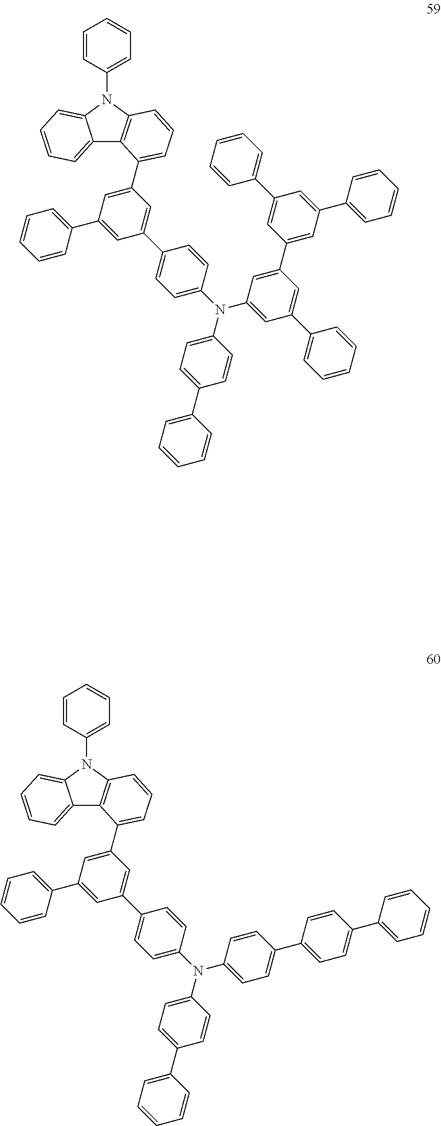 [SMILES: c1ccc(-c2ccc(N(c3ccc(-c4cc(-c5ccccc5)cc(-c5cccc6c5c5ccccc5n6-c5ccccc5)c4)cc3)c3cc(-c4ccccc4)cc(-c4cc(-c5ccccc5)cc(-c5ccccc5)c4)c3)cc2)cc1 ; c1ccc(-c2ccc(-c3ccc(N(c4ccc(-c5ccccc5)cc4)c4ccc(-c5cc(-c6ccccc6)cc(-c6cccc7c6c6ccccc6n7-c6ccccc6)c5)cc4)cc3)cc2)cc1]

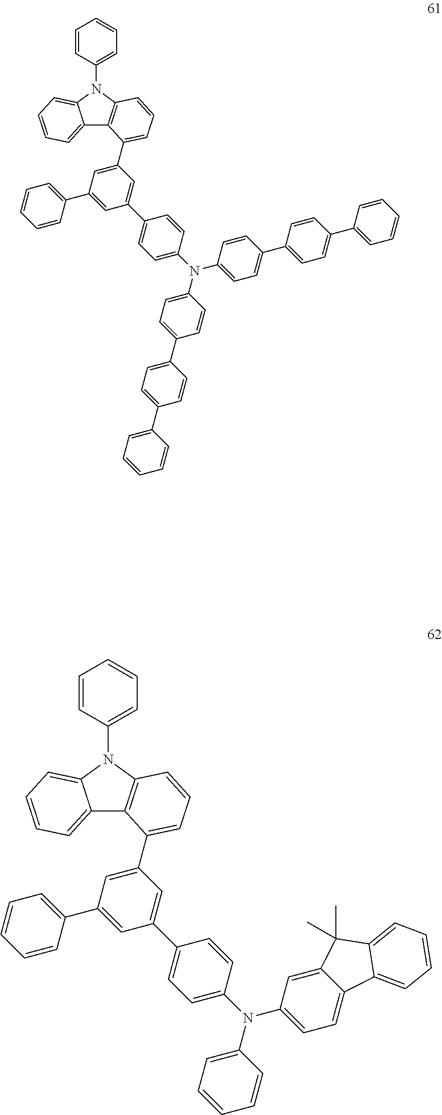 [SMILES: c1ccc(-c2ccc(-c3ccc(N(c4ccc(-c5ccc(-c6ccccc6)cc5)cc4)c4ccc(-c5cc(-c6ccccc6)cc(-c6cccc7c6c6ccccc6n7-c6ccccc6)c5)cc4)cc3)cc2)cc1 ; CC1(C)c2ccccc2-c2ccc(N(c3ccccc3)c3ccc(-c4cc(-c5ccccc5)cc(-c5cccc6c5c5ccccc5n6-c5ccccc5)c4)cc3)cc21]

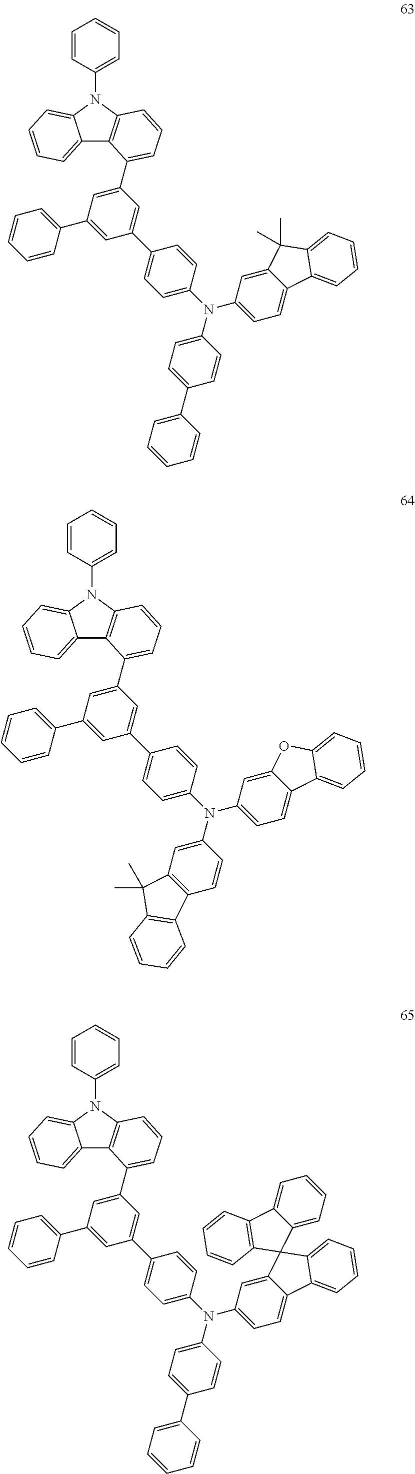 [SMILES: CC1(C)c2ccccc2-c2ccc(N(c3ccc(-c4ccccc4)cc3)c3ccc(-c4cc(-c5ccccc5)cc(-c5cccc6c5c5ccccc5n6-c5ccccc5)c4)cc3)cc21 ; CC1(C)c2ccccc2-c2ccc(N(c3ccc(-c4cc(-c5ccccc5)cc(-c5cccc6c5c5ccccc5n6-c5ccccc5)c4)cc3)c3ccc4c(c3)oc3ccccc34)cc21 ; c1ccc(-c2ccc(N(c3ccc(-c4cc(-c5ccccc5)cc(-c5cccc6c5c5ccccc5n6-c5ccccc5)c4)cc3)c3ccc4c(c3)C3(c5ccccc5-c5ccccc53)c3ccccc3-4)cc2)cc1]

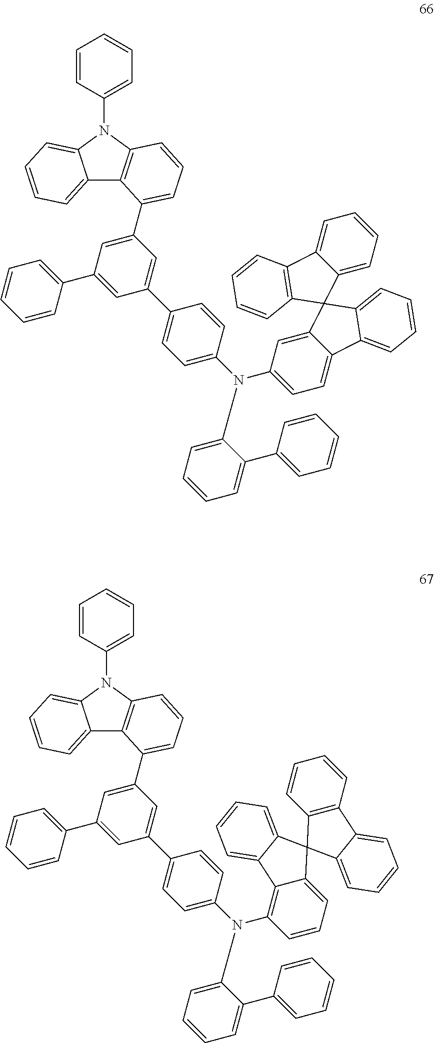 [SMILES: c1ccc(-c2cc(-c3ccc(N(c4ccc5c(c4)C4(c6ccccc6-c6ccccc64)c4ccccc4-5)c4ccccc4-c4ccccc4)cc3)cc(-c3cccc4c3c3ccccc3n4-c3ccccc3)c2)cc1 ; c1ccc(-c2cc(-c3ccc(N(c4ccccc4-c4ccccc4)c4cccc5c4-c4ccccc4C54c5ccccc5-c5ccccc54)cc3)cc(-c3cccc4c3c3ccccc3n4-c3ccccc3)c2)cc1]

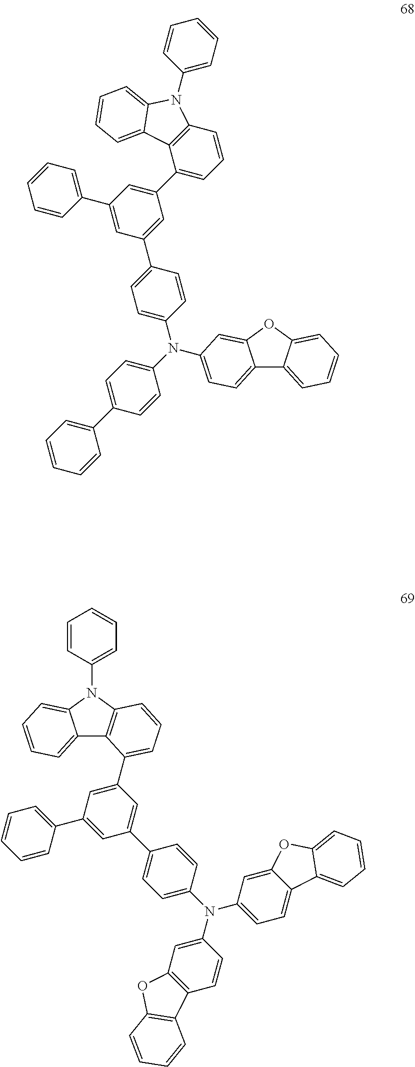 [SMILES: c1ccc(-c2ccc(N(c3ccc(-c4cc(-c5ccccc5)cc(-c5cccc6c5c5ccccc5n6-c5ccccc5)c4)cc3)c3ccc4c(c3)oc3ccccc34)cc2)cc1 ; c1ccc(-c2cc(-c3ccc(N(c4ccc5c(c4)oc4ccccc45)c4ccc5c(c4)oc4ccccc45)cc3)cc(-c3cccc4c3c3ccccc3n4-c3ccccc3)c2)cc1]

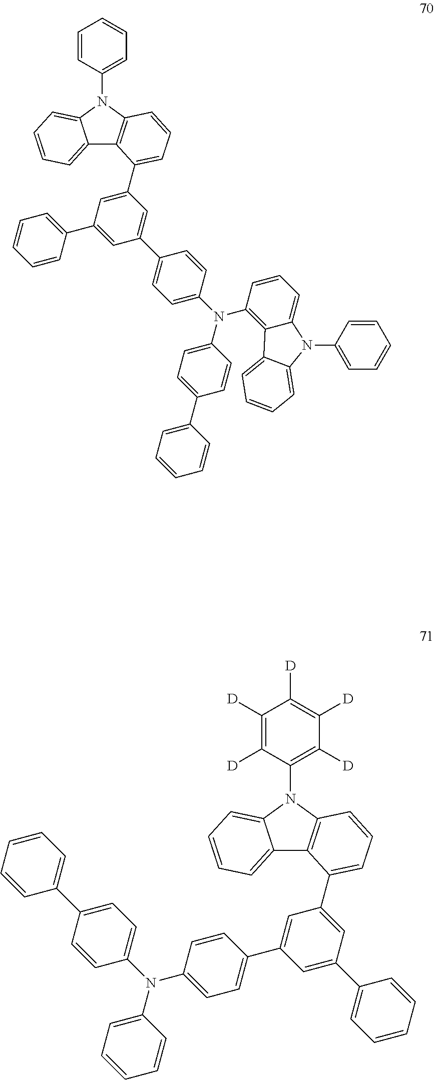 [SMILES: c1ccc(-c2ccc(N(c3ccc(-c4cc(-c5ccccc5)cc(-c5cccc6c5c5ccccc5n6-c5ccccc5)c4)cc3)c3cccc4c3c3ccccc3n4-c3ccccc3)cc2)cc1 ; [2H]c1c([2H])c([2H])c(-n2c3ccccc3c3c(-c4cc(-c5ccccc5)cc(-c5ccc(N(c6ccccc6)c6ccc(-c7ccccc7)cc6)cc5)c4)cccc32)c([2H])c1[2H]]

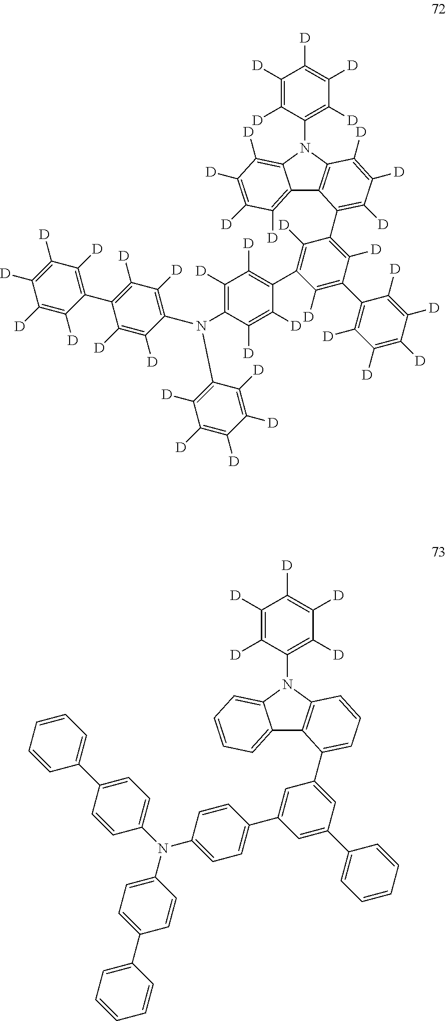 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c(-c5c([2H])c([2H])c([2H])c([2H])c5[2H])c([2H])c(-c5c([2H])c([2H])c([2H])c6c5c5c([2H])c([2H])c([2H])c([2H])c5n6-c5c([2H])c([2H])c([2H])c([2H])c5[2H])c4[2H])c([2H])c3[2H])c([2H])c2[2H])c([2H])c1[2H] ; [2H]c1c([2H])c([2H])c(-n2c3ccccc3c3c(-c4cc(-c5ccccc5)cc(-c5ccc(N(c6ccc(-c7ccccc7)cc6)c6ccc(-c7ccccc7)cc6)cc5)c4)cccc32)c([2H])c1[2H]]

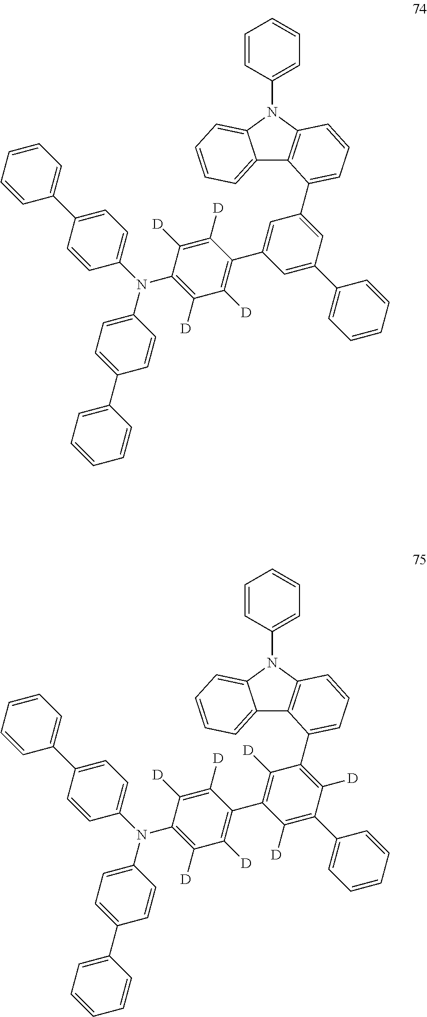 [SMILES: [2H]c1c([2H])c(N(c2ccc(-c3ccccc3)cc2)c2ccc(-c3ccccc3)cc2)c([2H])c([2H])c1-c1cc(-c2ccccc2)cc(-c2cccc3c2c2ccccc2n3-c2ccccc2)c1 ; [2H]c1c([2H])c(N(c2ccc(-c3ccccc3)cc2)c2ccc(-c3ccccc3)cc2)c([2H])c([2H])c1-c1c([2H])c(-c2ccccc2)c([2H])c(-c2cccc3c2c2ccccc2n3-c2ccccc2)c1[2H]]

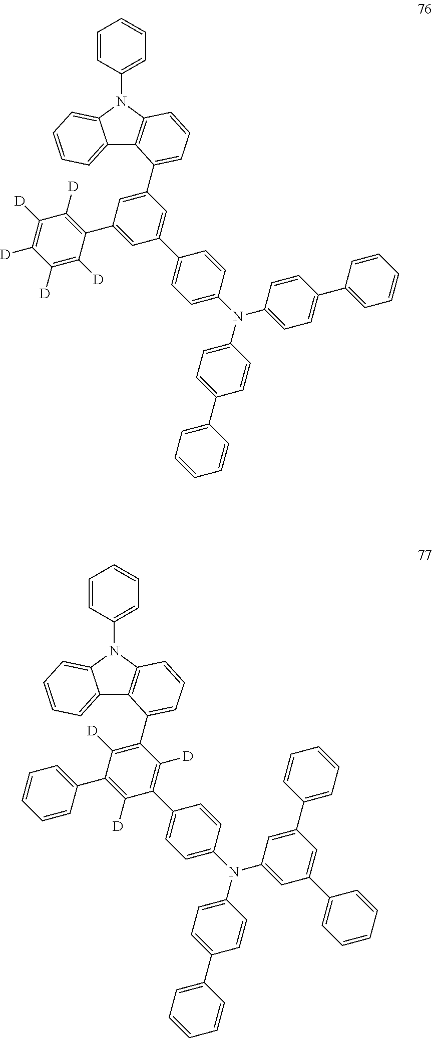 [SMILES: [2H]c1c([2H])c([2H])c(-c2cc(-c3ccc(N(c4ccc(-c5ccccc5)cc4)c4ccc(-c5ccccc5)cc4)cc3)cc(-c3cccc4c3c3ccccc3n4-c3ccccc3)c2)c([2H])c1[2H] ; [2H]c1c(-c2ccccc2)c([2H])c(-c2cccc3c2c2ccccc2n3-c2ccccc2)c([2H])c1-c1ccc(N(c2ccc(-c3ccccc3)cc2)c2cc(-c3ccccc3)cc(-c3ccccc3)c2)cc1]

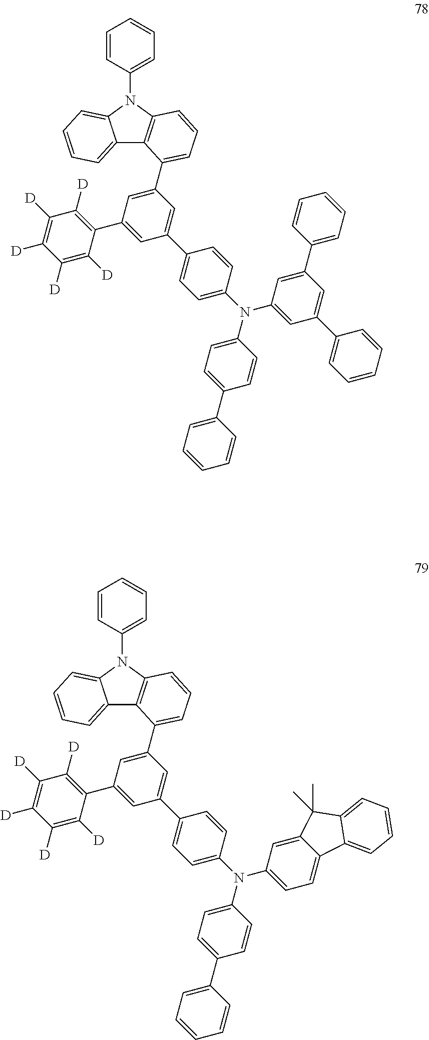 [SMILES: [2H]c1c([2H])c([2H])c(-c2cc(-c3ccc(N(c4ccc(-c5ccccc5)cc4)c4cc(-c5ccccc5)cc(-c5ccccc5)c4)cc3)cc(-c3cccc4c3c3ccccc3n4-c3ccccc3)c2)c([2H])c1[2H] ; [2H]c1c([2H])c([2H])c(-c2cc(-c3ccc(N(c4ccc(-c5ccccc5)cc4)c4ccc5c(c4)C(C)(C)c4ccccc4-5)cc3)cc(-c3cccc4c3c3ccccc3n4-c3ccccc3)c2)c([2H])c1[2H]]

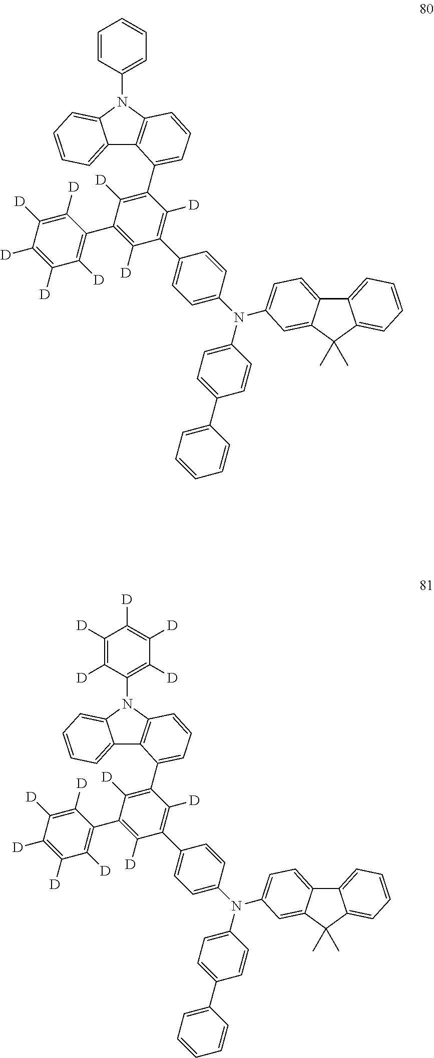 [SMILES: [2H]c1c([2H])c([2H])c(-c2c([2H])c(-c3ccc(N(c4ccc(-c5ccccc5)cc4)c4ccc5c(c4)C(C)(C)c4ccccc4-5)cc3)c([2H])c(-c3cccc4c3c3ccccc3n4-c3ccccc3)c2[2H])c([2H])c1[2H] ; [2H]c1c([2H])c([2H])c(-c2c([2H])c(-c3ccc(N(c4ccc(-c5ccccc5)cc4)c4ccc5c(c4)C(C)(C)c4ccccc4-5)cc3)c([2H])c(-c3cccc4c3c3ccccc3n4-c3c([2H])c([2H])c([2H])c([2H])c3[2H])c2[2H])c([2H])c1[2H]]

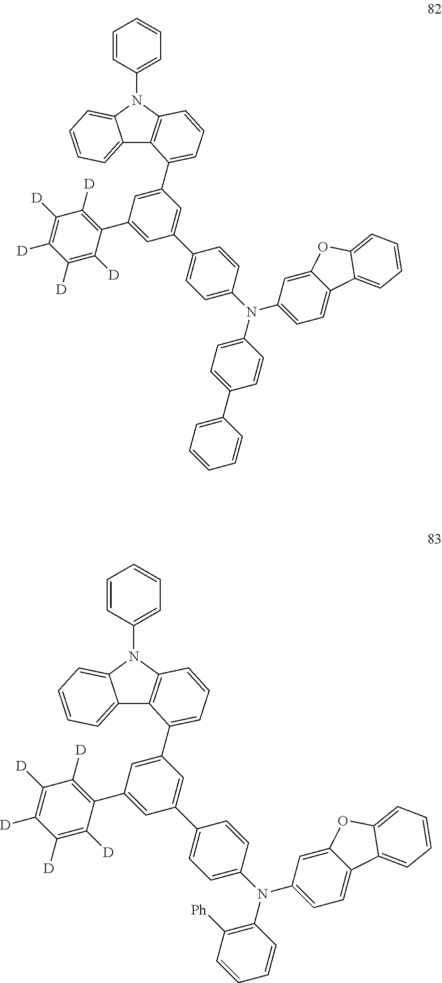 [SMILES: [2H]c1c([2H])c([2H])c(-c2cc(-c3ccc(N(c4ccc(-c5ccccc5)cc4)c4ccc5c(c4)oc4ccccc45)cc3)cc(-c3cccc4c3c3ccccc3n4-c3ccccc3)c2)c([2H])c1[2H] ; [2H]c1c([2H])c([2H])c(-c2cc(-c3ccc(N(c4ccc5c(c4)oc4ccccc45)c4ccccc4-c4ccccc4)cc3)cc(-c3cccc4c3c3ccccc3n4-c3ccccc3)c2)c([2H])c1[2H]]

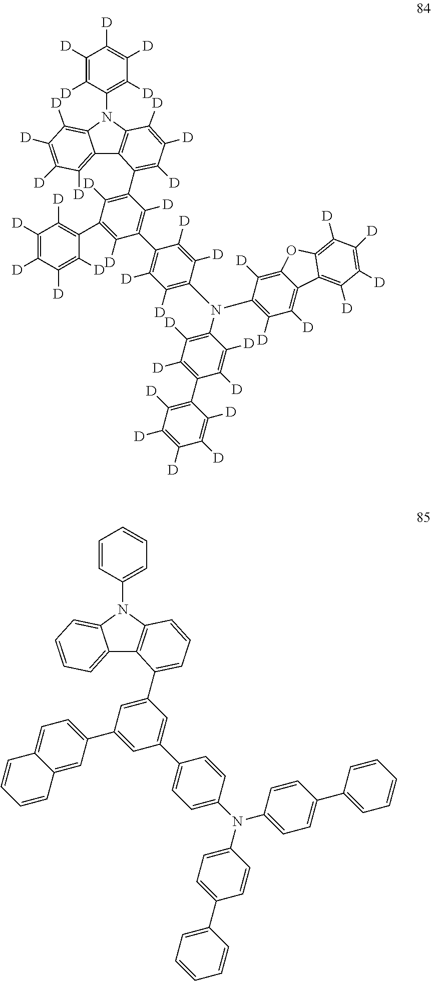 [SMILES: [2H]c1c([2H])c([2H])c(-c2c([2H])c([2H])c(N(c3c([2H])c([2H])c(-c4c([2H])c(-c5c([2H])c([2H])c([2H])c([2H])c5[2H])c([2H])c(-c5c([2H])c([2H])c([2H])c6c5c5c([2H])c([2H])c([2H])c([2H])c5n6-c5c([2H])c([2H])c([2H])c([2H])c5[2H])c4[2H])c([2H])c3[2H])c3c([2H])c([2H])c4c(oc5c([2H])c([2H])c([2H])c([2H])c54)c3[2H])c([2H])c2[2H])c([2H])c1[2H] ; c1ccc(-c2ccc(N(c3ccc(-c4ccccc4)cc3)c3ccc(-c4cc(-c5ccc6ccccc6c5)cc(-c5cccc6c5c5ccccc5n6-c5ccccc5)c4)cc3)cc2)cc1]

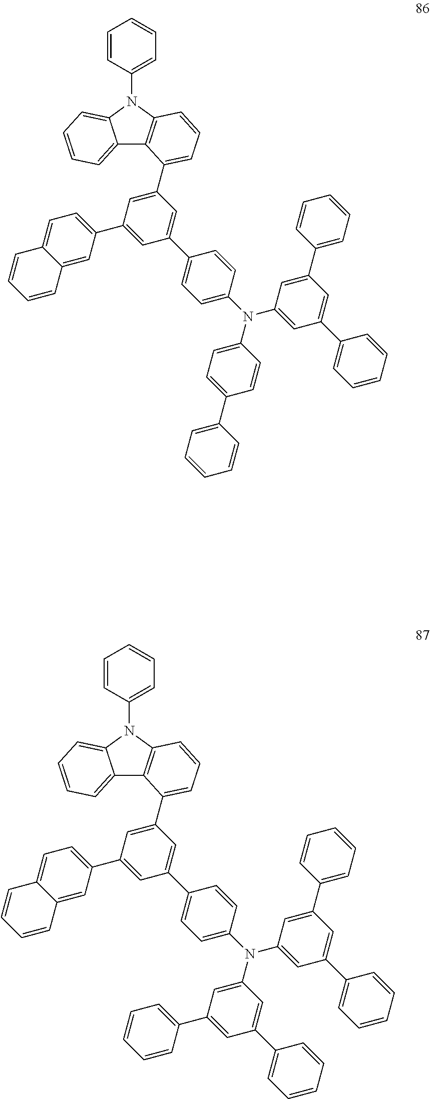 [SMILES: c1ccc(-c2ccc(N(c3ccc(-c4cc(-c5ccc6ccccc6c5)cc(-c5cccc6c5c5ccccc5n6-c5ccccc5)c4)cc3)c3cc(-c4ccccc4)cc(-c4ccccc4)c3)cc2)cc1 ; c1ccc(-c2cc(-c3ccccc3)cc(N(c3ccc(-c4cc(-c5ccc6ccccc6c5)cc(-c5cccc6c5c5ccccc5n6-c5ccccc5)c4)cc3)c3cc(-c4ccccc4)cc(-c4ccccc4)c3)c2)cc1]

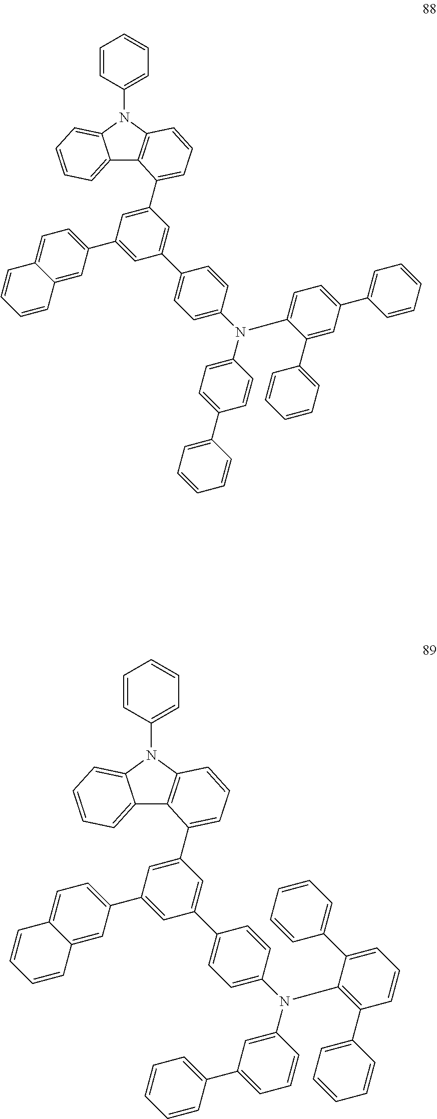 [SMILES: c1ccc(-c2ccc(N(c3ccc(-c4cc(-c5ccc6ccccc6c5)cc(-c5cccc6c5c5ccccc5n6-c5ccccc5)c4)cc3)c3ccc(-c4ccccc4)cc3-c3ccccc3)cc2)cc1 ; c1ccc(-c2cccc(N(c3ccc(-c4cc(-c5ccc6ccccc6c5)cc(-c5cccc6c5c5ccccc5n6-c5ccccc5)c4)cc3)c3c(-c4ccccc4)cccc3-c3ccccc3)c2)cc1]

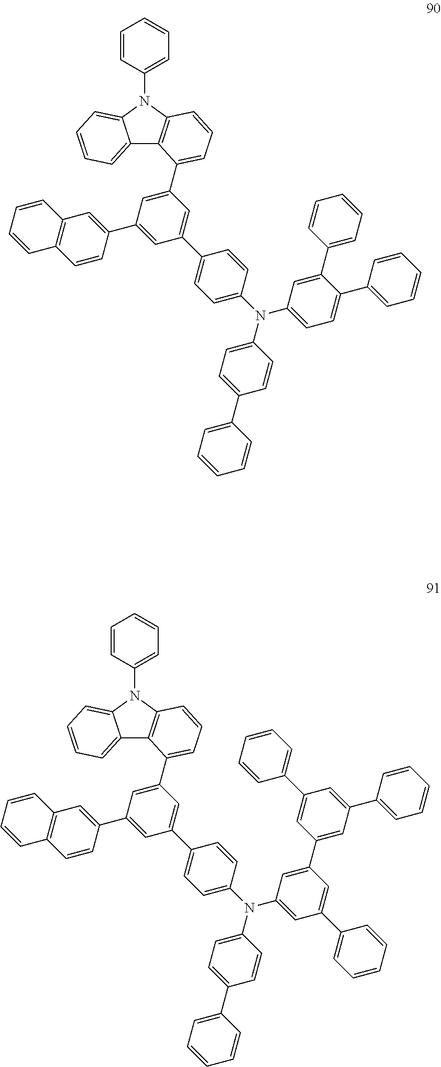 [SMILES: c1ccc(-c2ccc(N(c3ccc(-c4cc(-c5ccc6ccccc6c5)cc(-c5cccc6c5c5ccccc5n6-c5ccccc5)c4)cc3)c3ccc(-c4ccccc4)c(-c4ccccc4)c3)cc2)cc1 ; c1ccc(-c2ccc(N(c3ccc(-c4cc(-c5ccc6ccccc6c5)cc(-c5cccc6c5c5ccccc5n6-c5ccccc5)c4)cc3)c3cc(-c4ccccc4)cc(-c4cc(-c5ccccc5)cc(-c5ccccc5)c4)c3)cc2)cc1]

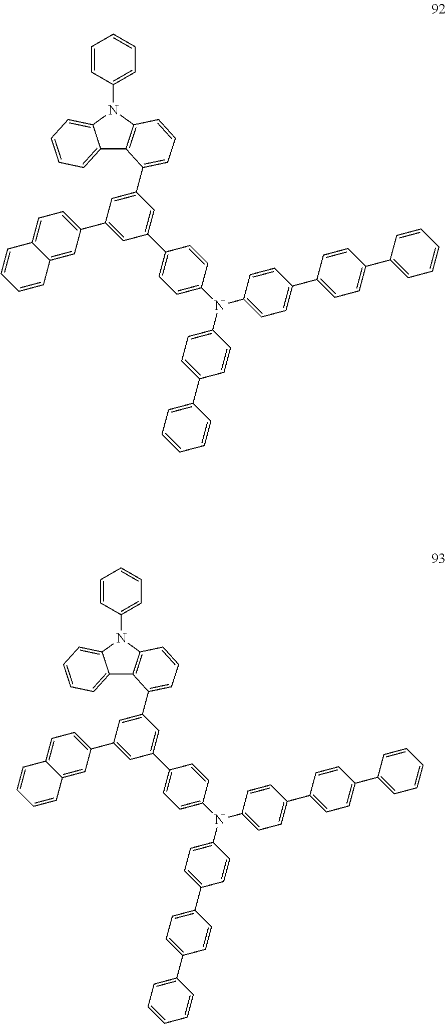 [SMILES: c1ccc(-c2ccc(-c3ccc(N(c4ccc(-c5ccccc5)cc4)c4ccc(-c5cc(-c6ccc7ccccc7c6)cc(-c6cccc7c6c6ccccc6n7-c6ccccc6)c5)cc4)cc3)cc2)cc1 ; c1ccc(-c2ccc(-c3ccc(N(c4ccc(-c5ccc(-c6ccccc6)cc5)cc4)c4ccc(-c5cc(-c6ccc7ccccc7c6)cc(-c6cccc7c6c6ccccc6n7-c6ccccc6)c5)cc4)cc3)cc2)cc1]

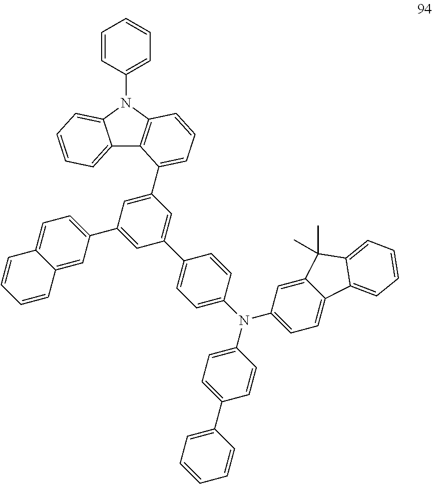 [SMILES: CC1(C)c2ccccc2-c2ccc(N(c3ccc(-c4ccccc4)cc3)c3ccc(-c4cc(-c5ccc6ccccc6c5)cc(-c5cccc6c5c5ccccc5n6-c5ccccc5)c4)cc3)cc21]

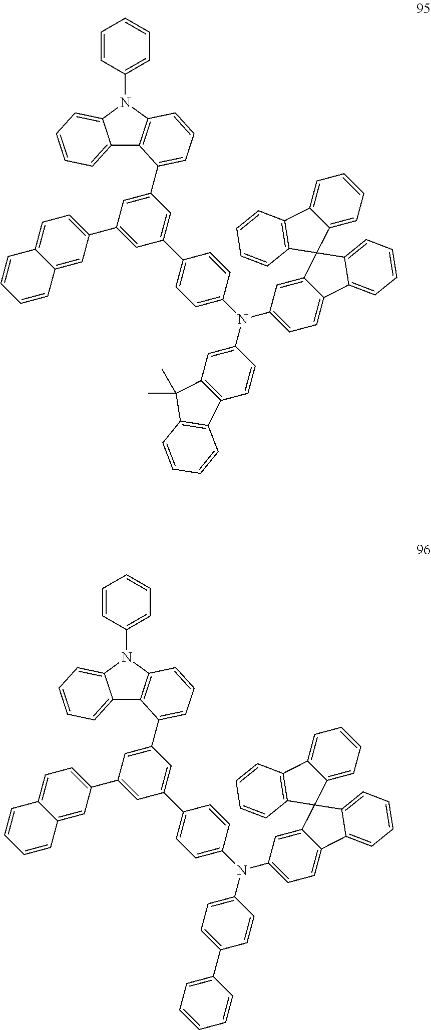 [SMILES: CC1(C)c2ccccc2-c2ccc(N(c3ccc(-c4cc(-c5ccc6ccccc6c5)cc(-c5cccc6c5c5ccccc5n6-c5ccccc5)c4)cc3)c3ccc4c(c3)C3(c5ccccc5-c5ccccc53)c3ccccc3-4)cc21 ; c1ccc(-c2ccc(N(c3ccc(-c4cc(-c5ccc6ccccc6c5)cc(-c5cccc6c5c5ccccc5n6-c5ccccc5)c4)cc3)c3ccc4c(c3)C3(c5ccccc5-c5ccccc53)c3ccccc3-4)cc2)cc1]

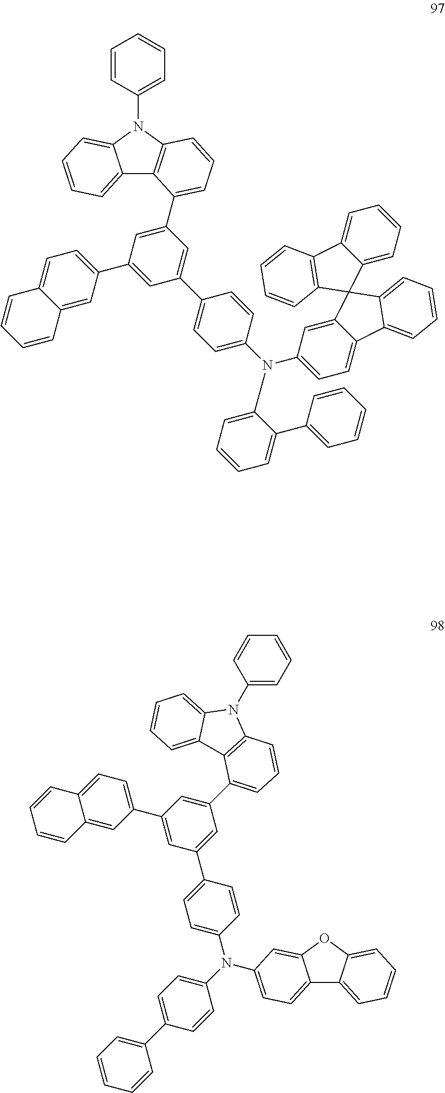 [SMILES: c1ccc(-c2ccccc2N(c2ccc(-c3cc(-c4ccc5ccccc5c4)cc(-c4cccc5c4c4ccccc4n5-c4ccccc4)c3)cc2)c2ccc3c(c2)C2(c4ccccc4-c4ccccc42)c2ccccc2-3)cc1 ; c1ccc(-c2ccc(N(c3ccc(-c4cc(-c5ccc6ccccc6c5)cc(-c5cccc6c5c5ccccc5n6-c5ccccc5)c4)cc3)c3ccc4c(c3)oc3ccccc34)cc2)cc1]

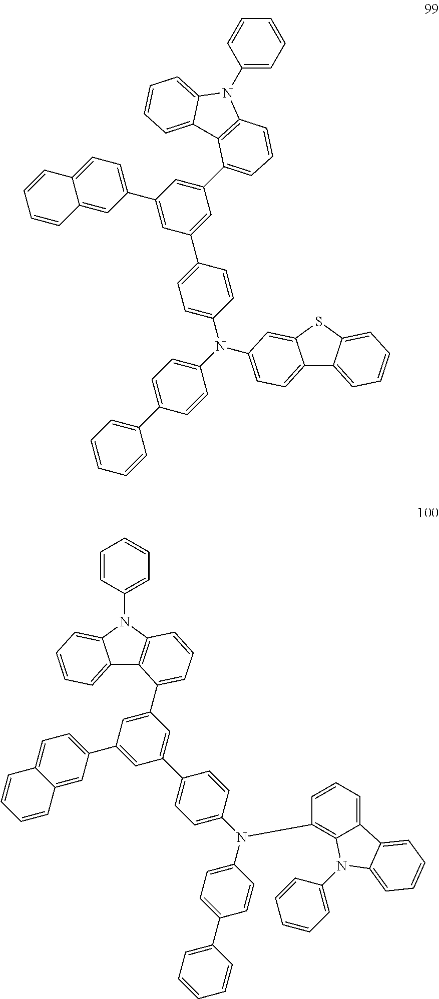 [SMILES: c1ccc(-c2ccc(N(c3ccc(-c4cc(-c5ccc6ccccc6c5)cc(-c5cccc6c5c5ccccc5n6-c5ccccc5)c4)cc3)c3ccc4c(c3)sc3ccccc34)cc2)cc1 ; c1ccc(-c2ccc(N(c3ccc(-c4cc(-c5ccc6ccccc6c5)cc(-c5cccc6c5c5ccccc5n6-c5ccccc5)c4)cc3)c3cccc4c5ccccc5n(-c5ccccc5)c34)cc2)cc1]

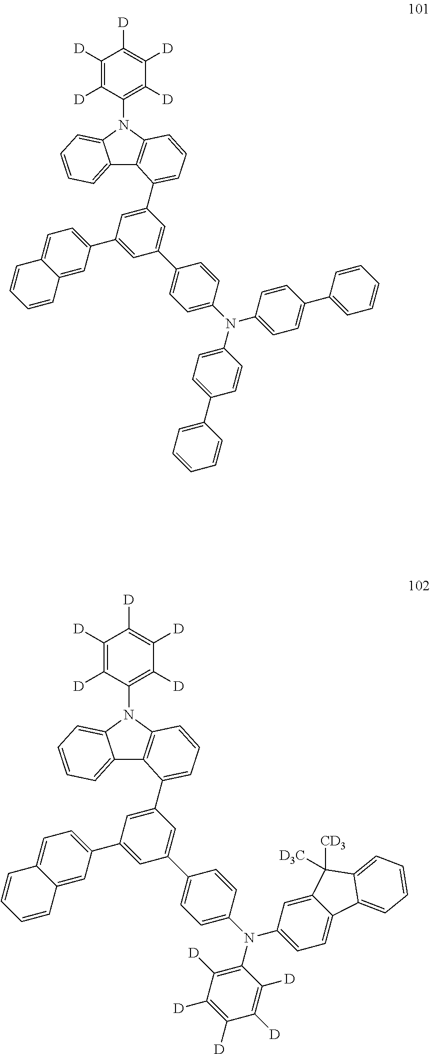 [SMILES: [2H]c1c([2H])c([2H])c(-n2c3ccccc3c3c(-c4cc(-c5ccc(N(c6ccc(-c7ccccc7)cc6)c6ccc(-c7ccccc7)cc6)cc5)cc(-c5ccc6ccccc6c5)c4)cccc32)c([2H])c1[2H] ; [2H]c1c([2H])c([2H])c(N(c2ccc(-c3cc(-c4ccc5ccccc5c4)cc(-c4cccc5c4c4ccccc4n5-c4c([2H])c([2H])c([2H])c([2H])c4[2H])c3)cc2)c2ccc3c(c2)C(C([2H])([2H])[2H])(C([2H])([2H])[2H])c2ccccc2-3)c([2H])c1[2H]]

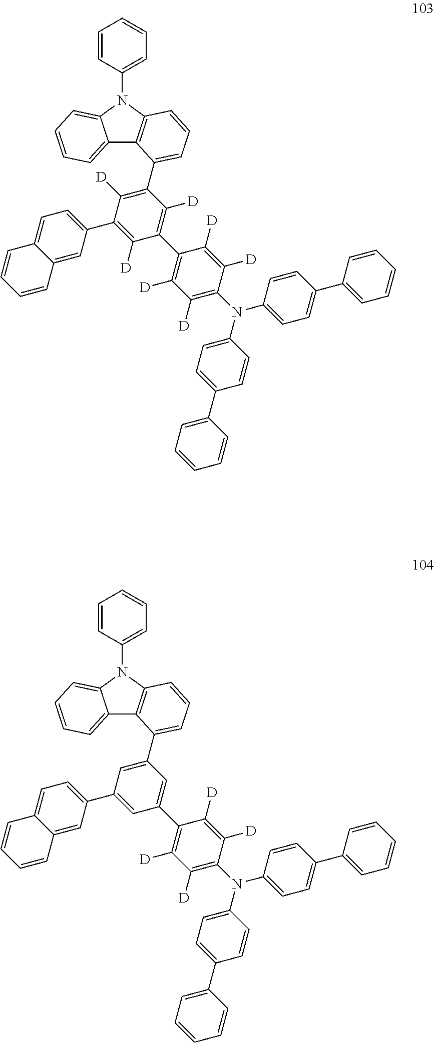 [SMILES: [2H]c1c([2H])c(N(c2ccc(-c3ccccc3)cc2)c2ccc(-c3ccccc3)cc2)c([2H])c([2H])c1-c1c([2H])c(-c2ccc3ccccc3c2)c([2H])c(-c2cccc3c2c2ccccc2n3-c2ccccc2)c1[2H] ; [2H]c1c([2H])c(N(c2ccc(-c3ccccc3)cc2)c2ccc(-c3ccccc3)cc2)c([2H])c([2H])c1-c1cc(-c2ccc3ccccc3c2)cc(-c2cccc3c2c2ccccc2n3-c2ccccc2)c1]

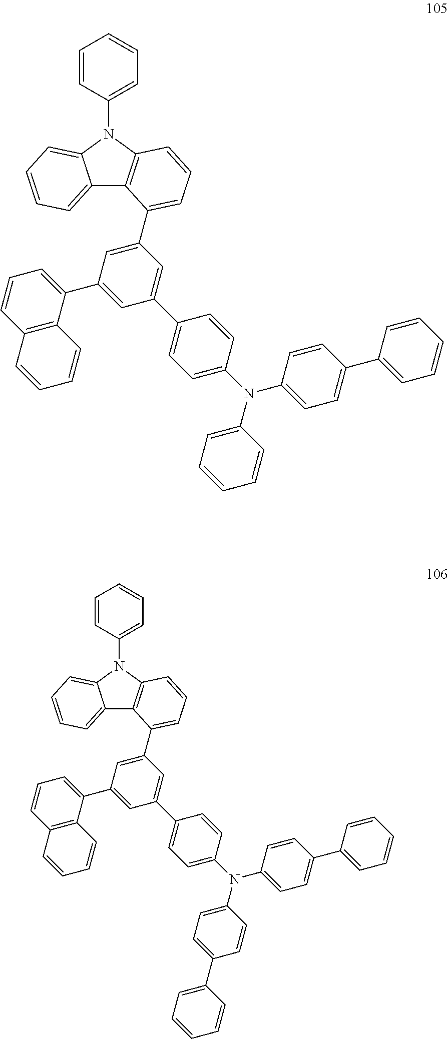 [SMILES: c1ccc(-c2ccc(N(c3ccccc3)c3ccc(-c4cc(-c5cccc6ccccc56)cc(-c5cccc6c5c5ccccc5n6-c5ccccc5)c4)cc3)cc2)cc1 ; c1ccc(-c2ccc(N(c3ccc(-c4ccccc4)cc3)c3ccc(-c4cc(-c5cccc6ccccc56)cc(-c5cccc6c5c5ccccc5n6-c5ccccc5)c4)cc3)cc2)cc1]

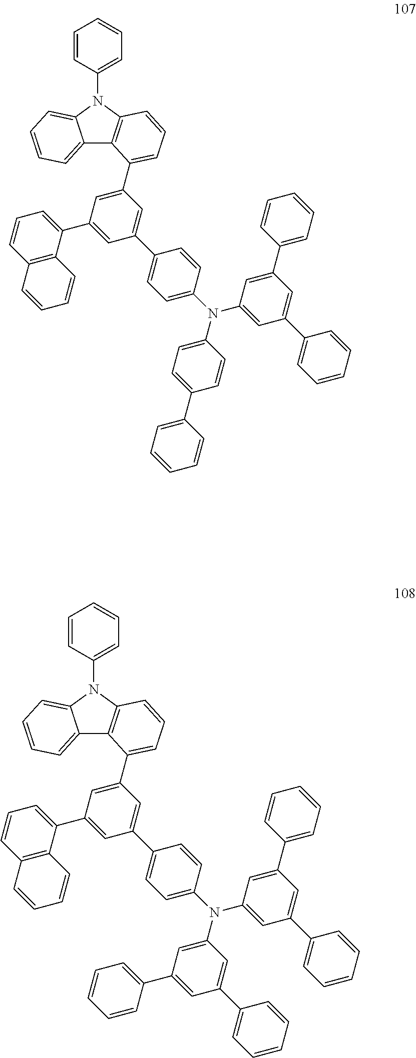 [SMILES: c1ccc(-c2ccc(N(c3ccc(-c4cc(-c5cccc6ccccc56)cc(-c5cccc6c5c5ccccc5n6-c5ccccc5)c4)cc3)c3cc(-c4ccccc4)cc(-c4ccccc4)c3)cc2)cc1 ; c1ccc(-c2cc(-c3ccccc3)cc(N(c3ccc(-c4cc(-c5cccc6ccccc56)cc(-c5cccc6c5c5ccccc5n6-c5ccccc5)c4)cc3)c3cc(-c4ccccc4)cc(-c4ccccc4)c3)c2)cc1]

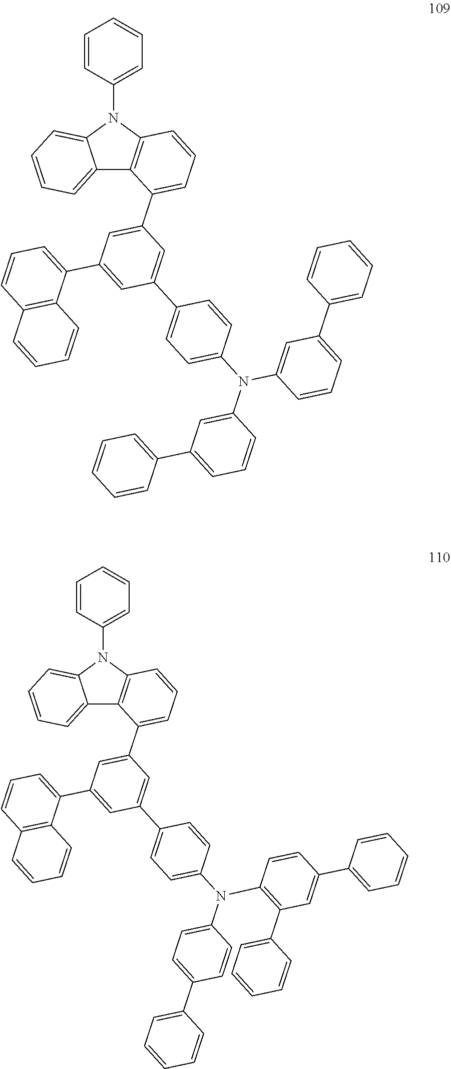 [SMILES: c1ccc(-c2cccc(N(c3ccc(-c4cc(-c5cccc6ccccc56)cc(-c5cccc6c5c5ccccc5n6-c5ccccc5)c4)cc3)c3cccc(-c4ccccc4)c3)c2)cc1 ; c1ccc(-c2ccc(N(c3ccc(-c4cc(-c5cccc6ccccc56)cc(-c5cccc6c5c5ccccc5n6-c5ccccc5)c4)cc3)c3ccc(-c4ccccc4)cc3-c3ccccc3)cc2)cc1]

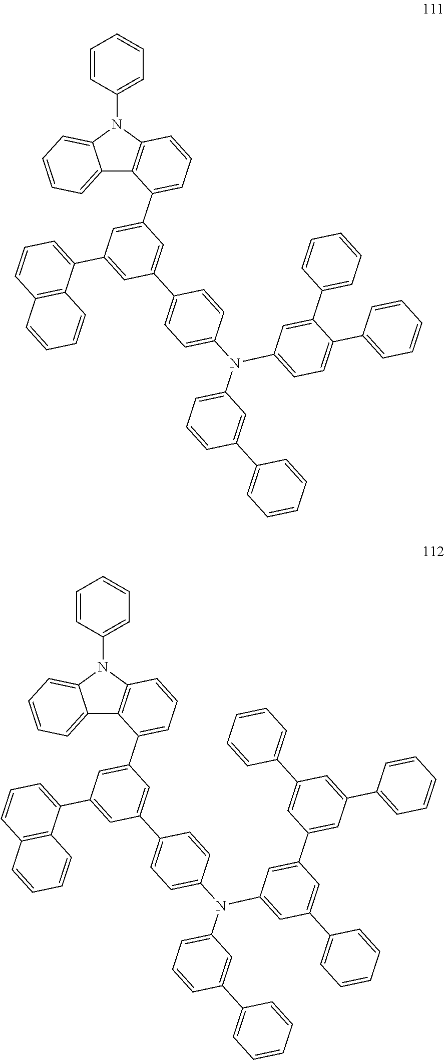 [SMILES: c1ccc(-c2cccc(N(c3ccc(-c4cc(-c5cccc6ccccc56)cc(-c5cccc6c5c5ccccc5n6-c5ccccc5)c4)cc3)c3ccc(-c4ccccc4)c(-c4ccccc4)c3)c2)cc1 ; c1ccc(-c2cc(-c3ccccc3)cc(-c3cc(-c4ccccc4)cc(N(c4ccc(-c5cc(-c6cccc7ccccc67)cc(-c6cccc7c6c6ccccc6n7-c6ccccc6)c5)cc4)c4cccc(-c5ccccc5)c4)c3)c2)cc1]

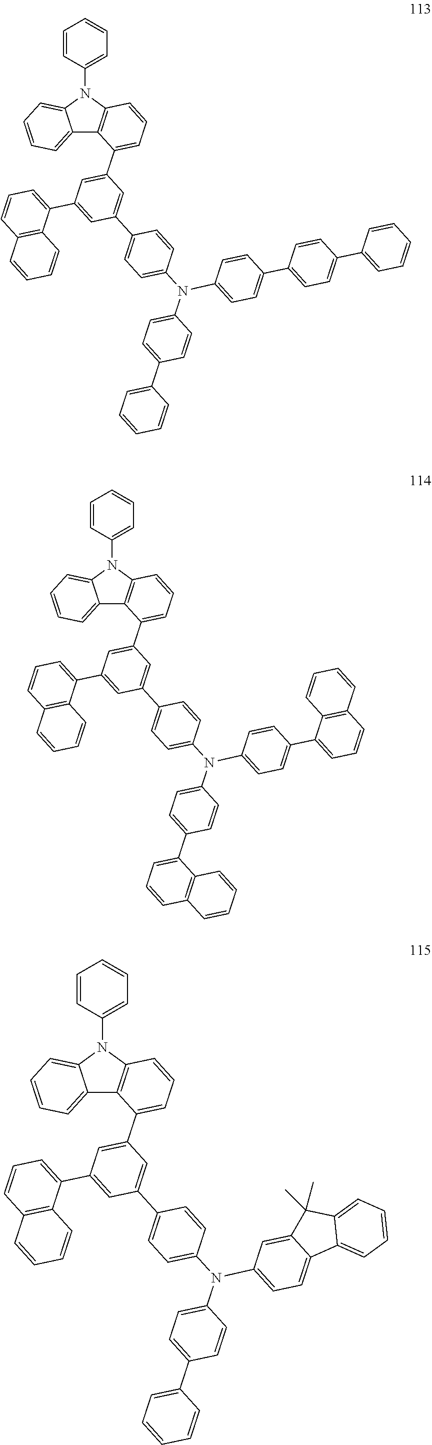 [SMILES: c1ccc(-c2ccc(-c3ccc(N(c4ccc(-c5ccccc5)cc4)c4ccc(-c5cc(-c6cccc7ccccc67)cc(-c6cccc7c6c6ccccc6n7-c6ccccc6)c5)cc4)cc3)cc2)cc1 ; c1ccc(-n2c3ccccc3c3c(-c4cc(-c5ccc(N(c6ccc(-c7cccc8ccccc78)cc6)c6ccc(-c7cccc8ccccc78)cc6)cc5)cc(-c5cccc6ccccc56)c4)cccc32)cc1 ; CC1(C)c2ccccc2-c2ccc(N(c3ccc(-c4ccccc4)cc3)c3ccc(-c4cc(-c5cccc6ccccc56)cc(-c5cccc6c5c5ccccc5n6-c5ccccc5)c4)cc3)cc21]

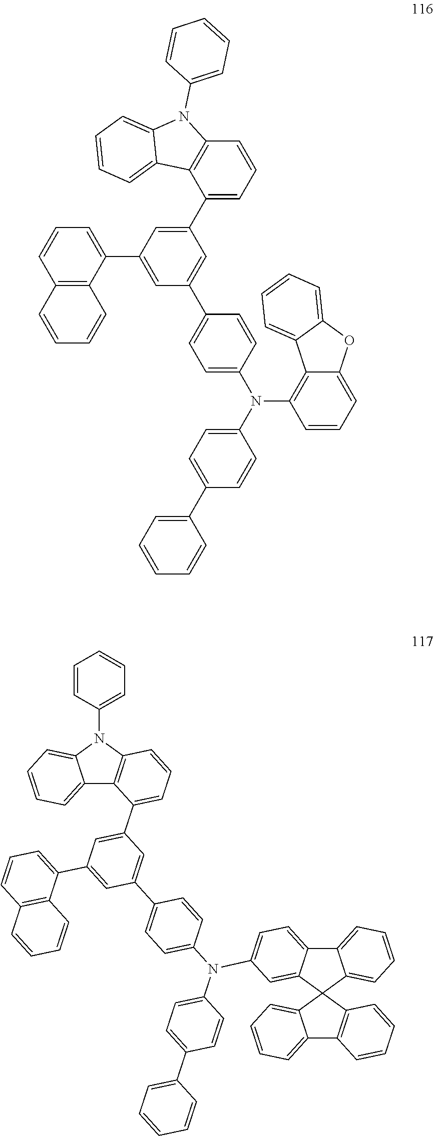 [SMILES: c1ccc(-c2ccc(N(c3ccc(-c4cc(-c5cccc6ccccc56)cc(-c5cccc6c5c5ccccc5n6-c5ccccc5)c4)cc3)c3cccc4oc5ccccc5c34)cc2)cc1 ; c1ccc(-c2ccc(N(c3ccc(-c4cc(-c5cccc6ccccc56)cc(-c5cccc6c5c5ccccc5n6-c5ccccc5)c4)cc3)c3ccc4c(c3)C3(c5ccccc5-c5ccccc53)c3ccccc3-4)cc2)cc1]

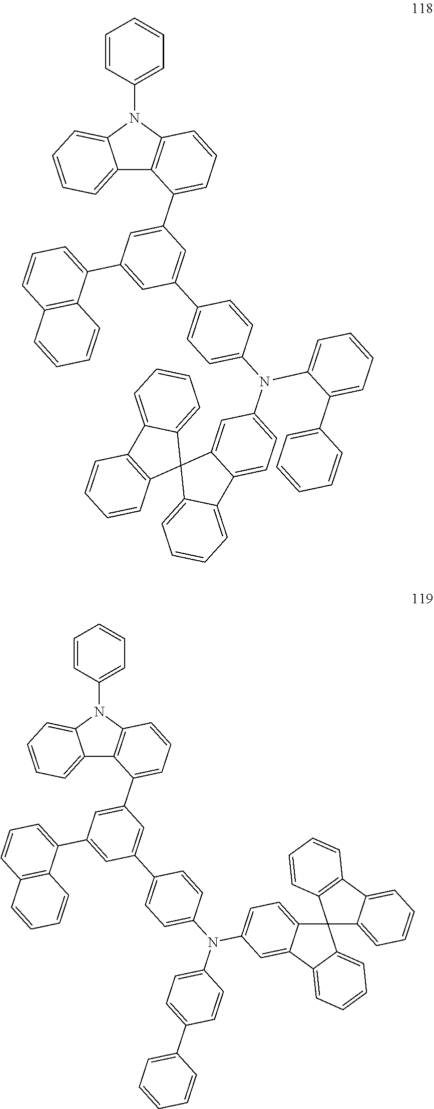 [SMILES: c1ccc(-c2ccccc2N(c2ccc(-c3cc(-c4cccc5ccccc45)cc(-c4cccc5c4c4ccccc4n5-c4ccccc4)c3)cc2)c2ccc3c(c2)C2(c4ccccc4-c4ccccc42)c2ccccc2-3)cc1 ; c1ccc(-c2ccc(N(c3ccc(-c4cc(-c5cccc6ccccc56)cc(-c5cccc6c5c5ccccc5n6-c5ccccc5)c4)cc3)c3ccc4c(c3)-c3ccccc3C43c4ccccc4-c4ccccc43)cc2)cc1]

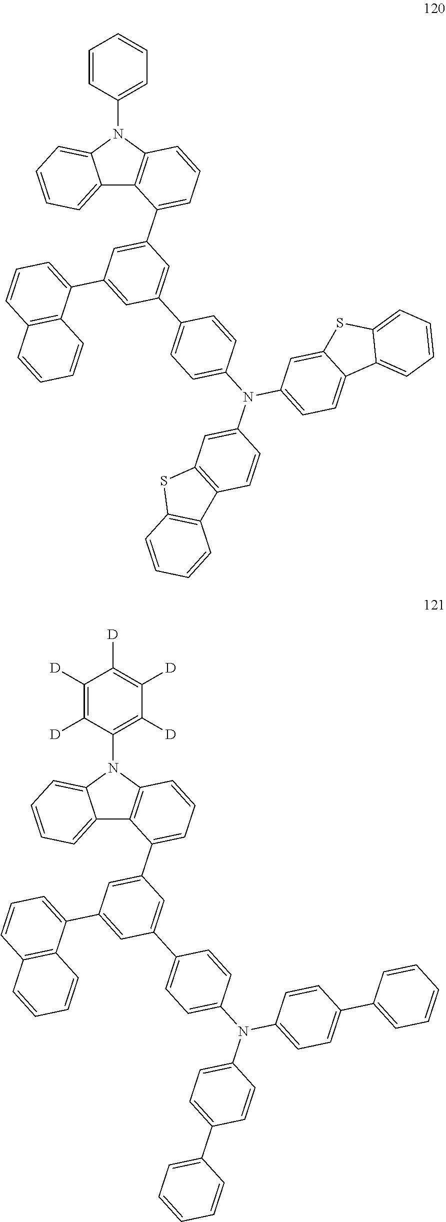 [SMILES: c1ccc(-n2c3ccccc3c3c(-c4cc(-c5ccc(N(c6ccc7c(c6)sc6ccccc67)c6ccc7c(c6)sc6ccccc67)cc5)cc(-c5cccc6ccccc56)c4)cccc32)cc1 ; [2H]c1c([2H])c([2H])c(-n2c3ccccc3c3c(-c4cc(-c5ccc(N(c6ccc(-c7ccccc7)cc6)c6ccc(-c7ccccc7)cc6)cc5)cc(-c5cccc6ccccc56)c4)cccc32)c([2H])c1[2H]]

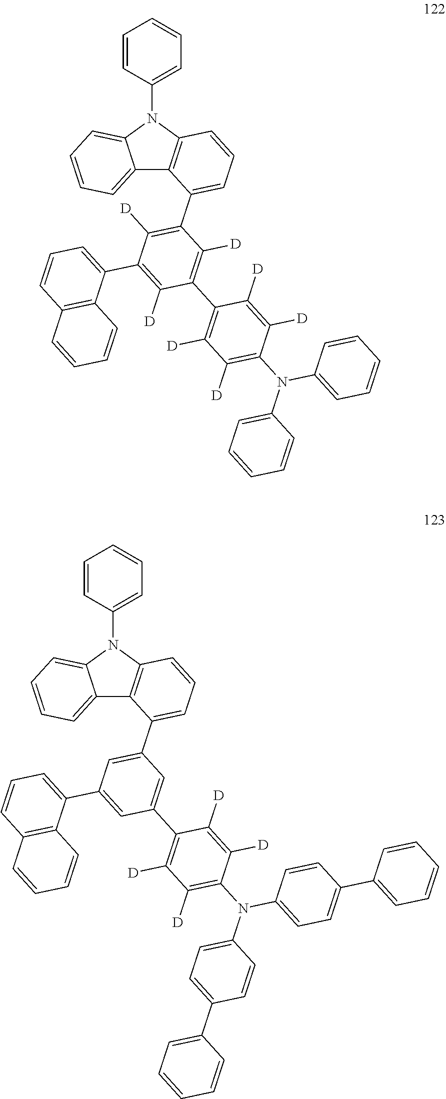 [SMILES: [2H]c1c([2H])c(N(c2ccccc2)c2ccccc2)c([2H])c([2H])c1-c1c([2H])c(-c2cccc3ccccc23)c([2H])c(-c2cccc3c2c2ccccc2n3-c2ccccc2)c1[2H] ; [2H]c1c([2H])c(N(c2ccc(-c3ccccc3)cc2)c2ccc(-c3ccccc3)cc2)c([2H])c([2H])c1-c1cc(-c2cccc3ccccc23)cc(-c2cccc3c2c2ccccc2n3-c2ccccc2)c1]

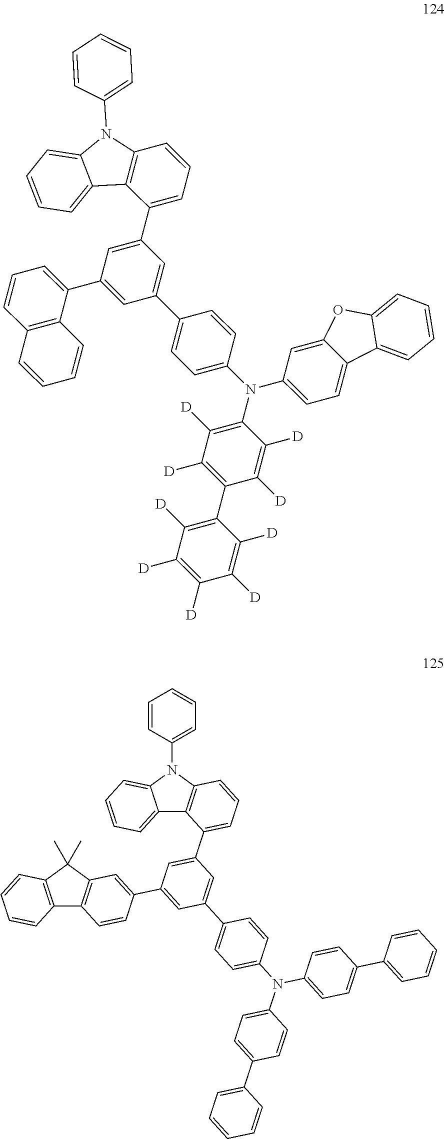 [SMILES: [2H]c1c([2H])c([2H])c(-c2c([2H])c([2H])c(N(c3ccc(-c4cc(-c5cccc6ccccc56)cc(-c5cccc6c5c5ccccc5n6-c5ccccc5)c4)cc3)c3ccc4c(c3)oc3ccccc34)c([2H])c2[2H])c([2H])c1[2H] ; CC1(C)c2ccccc2-c2ccc(-c3cc(-c4ccc(N(c5ccc(-c6ccccc6)cc5)c5ccc(-c6ccccc6)cc5)cc4)cc(-c4cccc5c4c4ccccc4n5-c4ccccc4)c3)cc21]

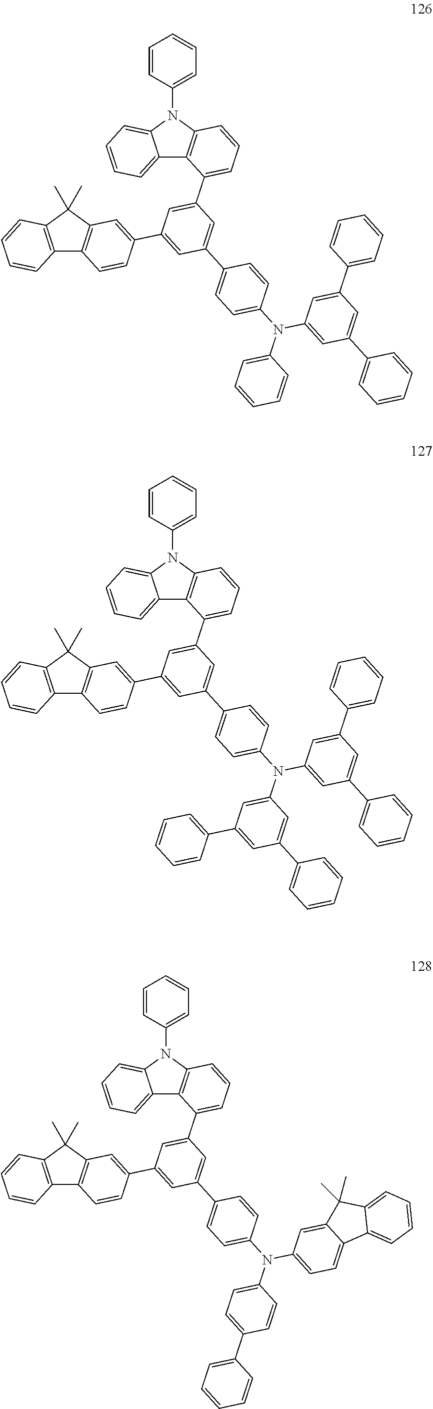 [SMILES: CC1(C)c2ccccc2-c2ccc(-c3cc(-c4ccc(N(c5ccccc5)c5cc(-c6ccccc6)cc(-c6ccccc6)c5)cc4)cc(-c4cccc5c4c4ccccc4n5-c4ccccc4)c3)cc21 ; CC1(C)c2ccccc2-c2ccc(-c3cc(-c4ccc(N(c5cc(-c6ccccc6)cc(-c6ccccc6)c5)c5cc(-c6ccccc6)cc(-c6ccccc6)c5)cc4)cc(-c4cccc5c4c4ccccc4n5-c4ccccc4)c3)cc21 ; CC1(C)c2ccccc2-c2ccc(-c3cc(-c4ccc(N(c5ccc(-c6ccccc6)cc5)c5ccc6c(c5)C(C)(C)c5ccccc5-6)cc4)cc(-c4cccc5c4c4ccccc4n5-c4ccccc4)c3)cc21]

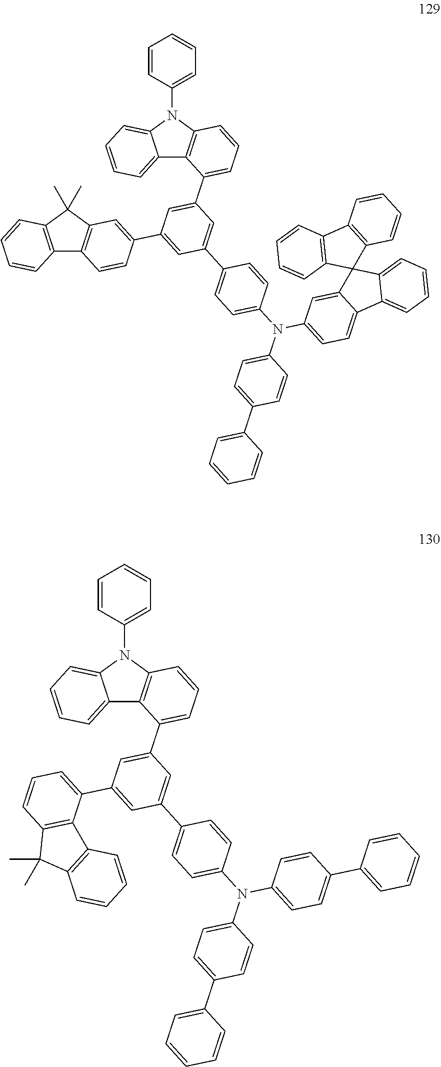 [SMILES: CC1(C)c2ccccc2-c2ccc(-c3cc(-c4ccc(N(c5ccc(-c6ccccc6)cc5)c5ccc6c(c5)C5(c7ccccc7-c7ccccc75)c5ccccc5-6)cc4)cc(-c4cccc5c4c4ccccc4n5-c4ccccc4)c3)cc21 ; CC1(C)c2ccccc2-c2c(-c3cc(-c4ccc(N(c5ccc(-c6ccccc6)cc5)c5ccc(-c6ccccc6)cc5)cc4)cc(-c4cccc5c4c4ccccc4n5-c4ccccc4)c3)cccc21]

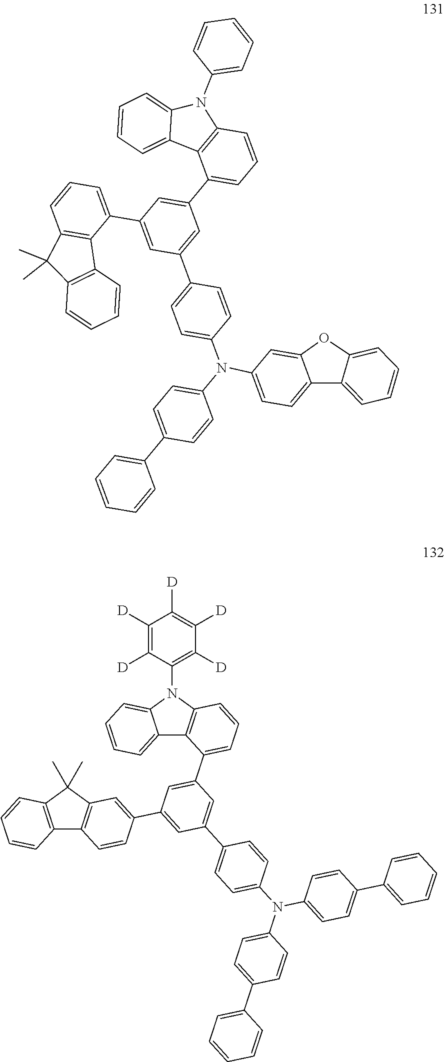 [SMILES: CC1(C)c2ccccc2-c2c(-c3cc(-c4ccc(N(c5ccc(-c6ccccc6)cc5)c5ccc6c(c5)oc5ccccc56)cc4)cc(-c4cccc5c4c4ccccc4n5-c4ccccc4)c3)cccc21 ; [2H]c1c([2H])c([2H])c(-n2c3ccccc3c3c(-c4cc(-c5ccc(N(c6ccc(-c7ccccc7)cc6)c6ccc(-c7ccccc7)cc6)cc5)cc(-c5ccc6c(c5)C(C)(C)c5ccccc5-6)c4)cccc32)c([2H])c1[2H]]

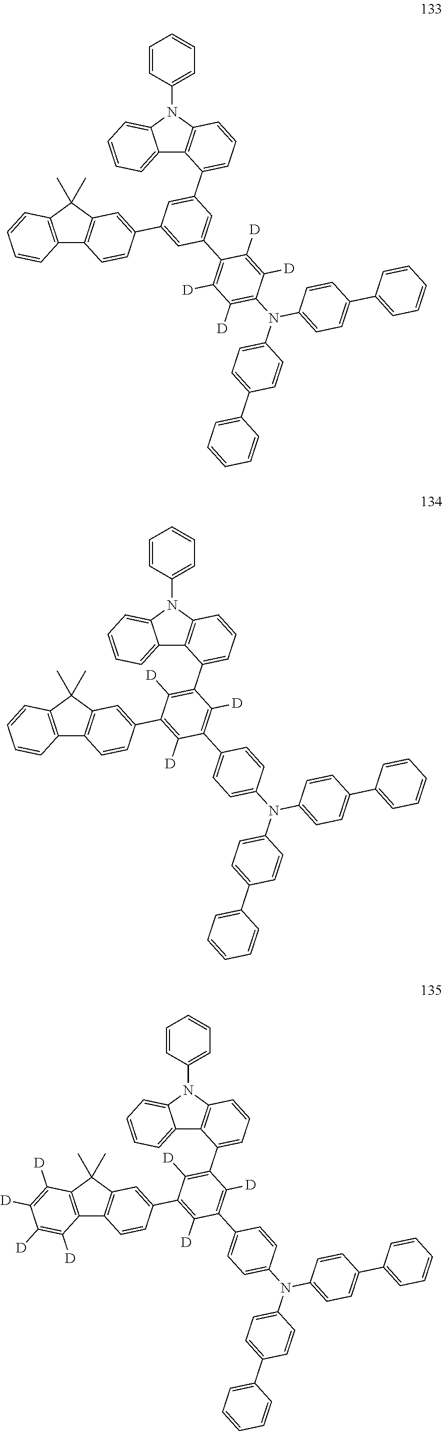 [SMILES: [2H]c1c([2H])c(N(c2ccc(-c3ccccc3)cc2)c2ccc(-c3ccccc3)cc2)c([2H])c([2H])c1-c1cc(-c2ccc3c(c2)C(C)(C)c2ccccc2-3)cc(-c2cccc3c2c2ccccc2n3-c2ccccc2)c1 ; [2H]c1c(-c2ccc(N(c3ccc(-c4ccccc4)cc3)c3ccc(-c4ccccc4)cc3)cc2)c([2H])c(-c2cccc3c2c2ccccc2n3-c2ccccc2)c([2H])c1-c1ccc2c(c1)C(C)(C)c1ccccc1-2 ; [2H]c1c([2H])c([2H])c2c(c1[2H])-c1ccc(-c3c([2H])c(-c4ccc(N(c5ccc(-c6ccccc6)cc5)c5ccc(-c6ccccc6)cc5)cc4)c([2H])c(-c4cccc5c4c4ccccc4n5-c4ccccc4)c3[2H])cc1C2(C)C]

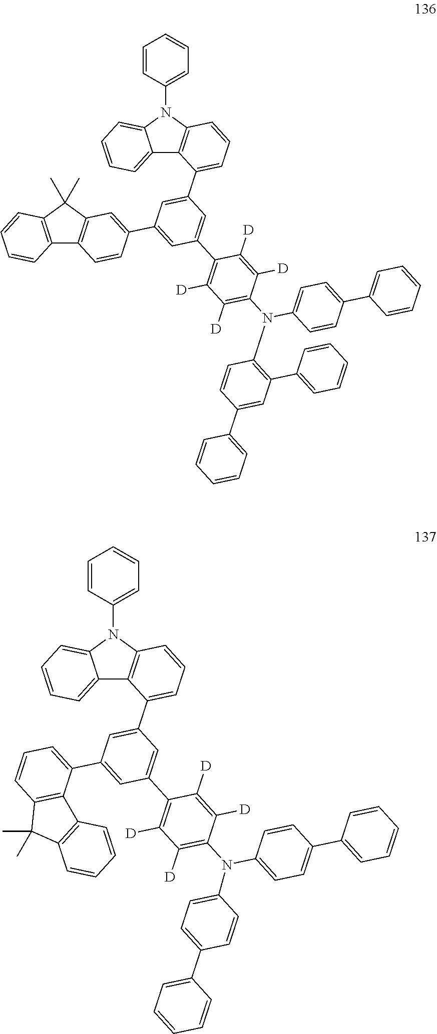 [SMILES: [2H]c1c([2H])c(N(c2ccc(-c3ccccc3)cc2)c2ccc(-c3ccccc3)cc2-c2ccccc2)c([2H])c([2H])c1-c1cc(-c2ccc3c(c2)C(C)(C)c2ccccc2-3)cc(-c2cccc3c2c2ccccc2n3-c2ccccc2)c1 ; [2H]c1c([2H])c(N(c2ccc(-c3ccccc3)cc2)c2ccc(-c3ccccc3)cc2)c([2H])c([2H])c1-c1cc(-c2cccc3c2-c2ccccc2C3(C)C)cc(-c2cccc3c2c2ccccc2n3-c2ccccc2)c1]

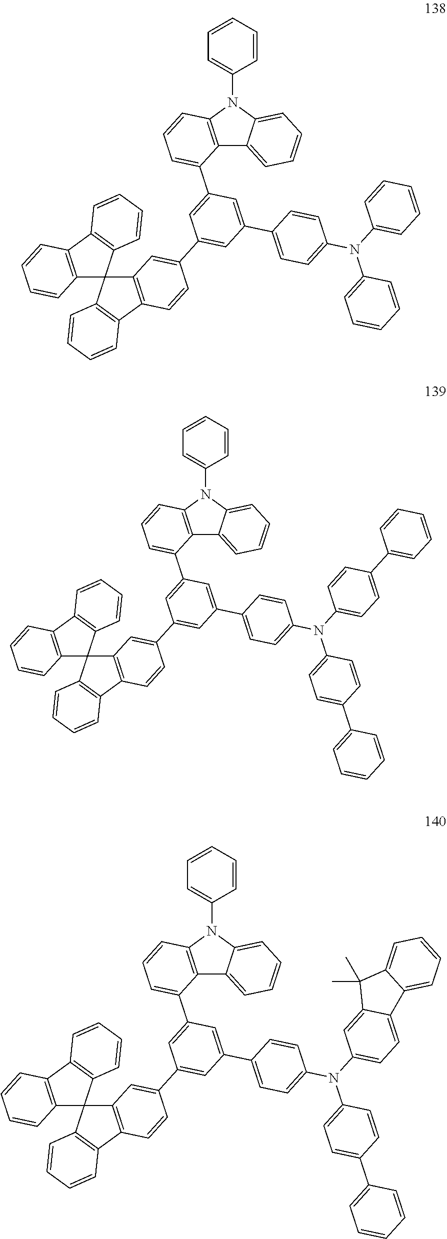 [SMILES: c1ccc(N(c2ccccc2)c2ccc(-c3cc(-c4ccc5c(c4)C4(c6ccccc6-c6ccccc64)c4ccccc4-5)cc(-c4cccc5c4c4ccccc4n5-c4ccccc4)c3)cc2)cc1 ; c1ccc(-c2ccc(N(c3ccc(-c4ccccc4)cc3)c3ccc(-c4cc(-c5ccc6c(c5)C5(c7ccccc7-c7ccccc75)c5ccccc5-6)cc(-c5cccc6c5c5ccccc5n6-c5ccccc5)c4)cc3)cc2)cc1 ; CC1(C)c2ccccc2-c2ccc(N(c3ccc(-c4ccccc4)cc3)c3ccc(-c4cc(-c5ccc6c(c5)C5(c7ccccc7-c7ccccc75)c5ccccc5-6)cc(-c5cccc6c5c5ccccc5n6-c5ccccc5)c4)cc3)cc21]

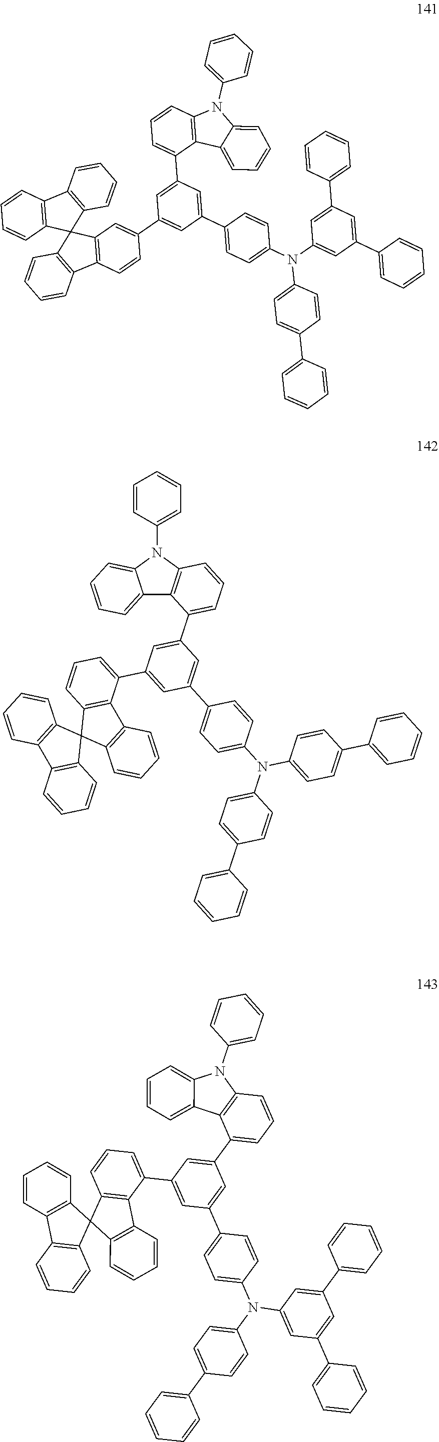 [SMILES: c1ccc(-c2ccc(N(c3ccc(-c4cc(-c5ccc6c(c5)C5(c7ccccc7-c7ccccc75)c5ccccc5-6)cc(-c5cccc6c5c5ccccc5n6-c5ccccc5)c4)cc3)c3cc(-c4ccccc4)cc(-c4ccccc4)c3)cc2)cc1 ; c1ccc(-c2ccc(N(c3ccc(-c4ccccc4)cc3)c3ccc(-c4cc(-c5cccc6c5-c5ccccc5C65c6ccccc6-c6ccccc65)cc(-c5cccc6c5c5ccccc5n6-c5ccccc5)c4)cc3)cc2)cc1 ; c1ccc(-c2ccc(N(c3ccc(-c4cc(-c5cccc6c5-c5ccccc5C65c6ccccc6-c6ccccc65)cc(-c5cccc6c5c5ccccc5n6-c5ccccc5)c4)cc3)c3cc(-c4ccccc4)cc(-c4ccccc4)c3)cc2)cc1]

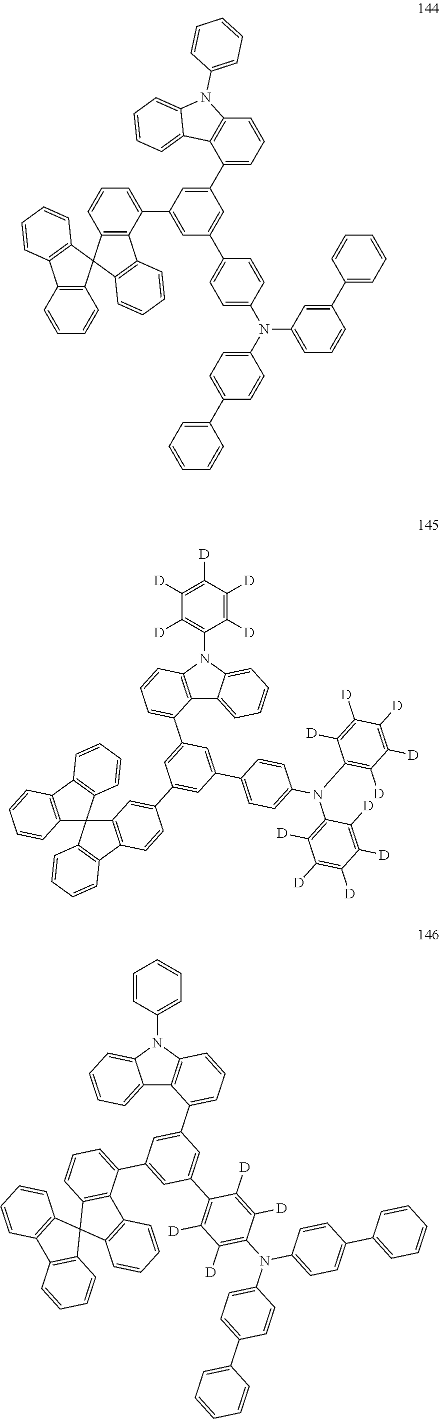 [SMILES: c1ccc(-c2ccc(N(c3ccc(-c4cc(-c5cccc6c5-c5ccccc5C65c6ccccc6-c6ccccc65)cc(-c5cccc6c5c5ccccc5n6-c5ccccc5)c4)cc3)c3cccc(-c4ccccc4)c3)cc2)cc1 ; [2H]c1c([2H])c([2H])c(N(c2ccc(-c3cc(-c4ccc5c(c4)C4(c6ccccc6-c6ccccc64)c4ccccc4-5)cc(-c4cccc5c4c4ccccc4n5-c4c([2H])c([2H])c([2H])c([2H])c4[2H])c3)cc2)c2c([2H])c([2H])c([2H])c([2H])c2[2H])c([2H])c1[2H] ; [2H]c1c([2H])c(N(c2ccc(-c3ccccc3)cc2)c2ccc(-c3ccccc3)cc2)c([2H])c([2H])c1-c1cc(-c2cccc3c2-c2ccccc2C32c3ccccc3-c3ccccc32)cc(-c2cccc3c2c2ccccc2n3-c2ccccc2)c1]

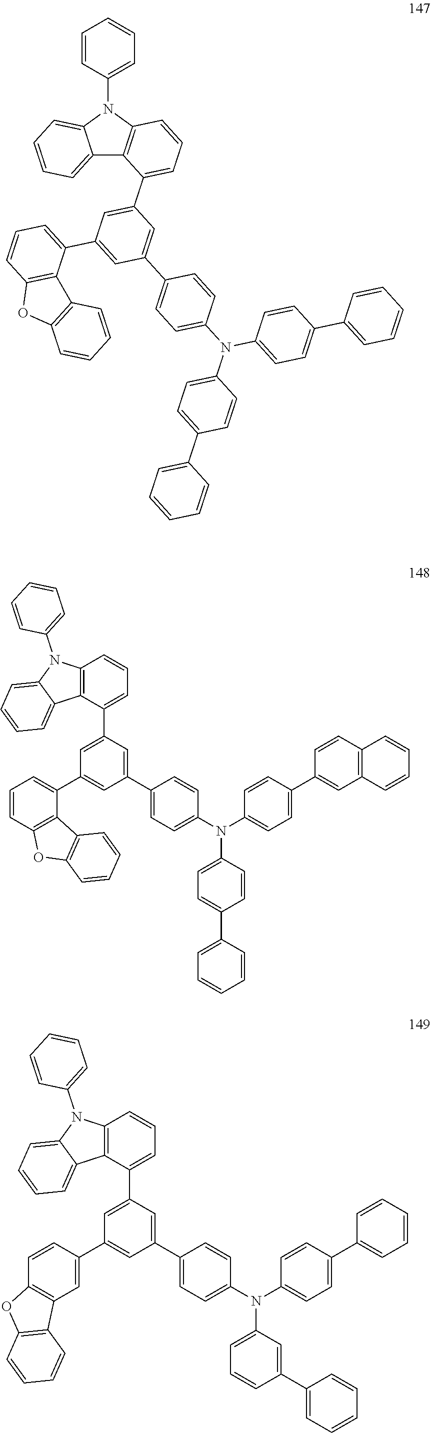 [SMILES: c1ccc(-c2ccc(N(c3ccc(-c4ccccc4)cc3)c3ccc(-c4cc(-c5cccc6oc7ccccc7c56)cc(-c5cccc6c5c5ccccc5n6-c5ccccc5)c4)cc3)cc2)cc1 ; c1ccc(-c2ccc(N(c3ccc(-c4cc(-c5cccc6oc7ccccc7c56)cc(-c5cccc6c5c5ccccc5n6-c5ccccc5)c4)cc3)c3ccc(-c4ccc5ccccc5c4)cc3)cc2)cc1 ; c1ccc(-c2ccc(N(c3ccc(-c4cc(-c5ccc6oc7ccccc7c6c5)cc(-c5cccc6c5c5ccccc5n6-c5ccccc5)c4)cc3)c3cccc(-c4ccccc4)c3)cc2)cc1]

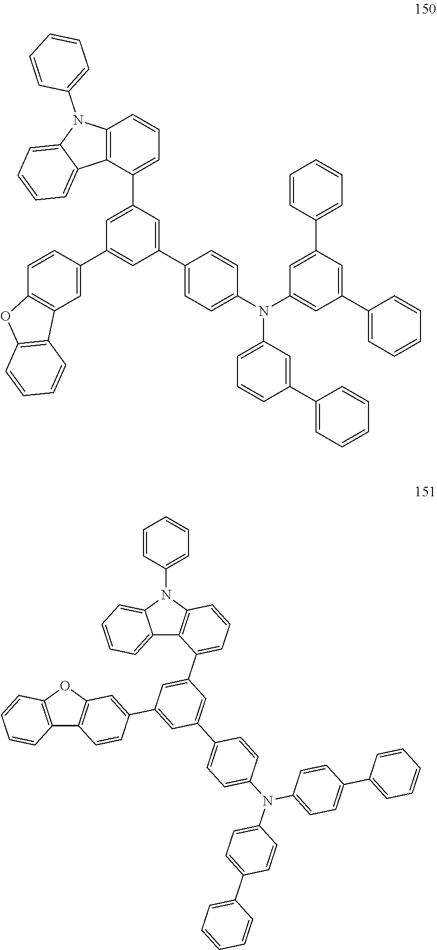 [SMILES: c1ccc(-c2cccc(N(c3ccc(-c4cc(-c5ccc6oc7ccccc7c6c5)cc(-c5cccc6c5c5ccccc5n6-c5ccccc5)c4)cc3)c3cc(-c4ccccc4)cc(-c4ccccc4)c3)c2)cc1 ; c1ccc(-c2ccc(N(c3ccc(-c4ccccc4)cc3)c3ccc(-c4cc(-c5ccc6c(c5)oc5ccccc56)cc(-c5cccc6c5c5ccccc5n6-c5ccccc5)c4)cc3)cc2)cc1]

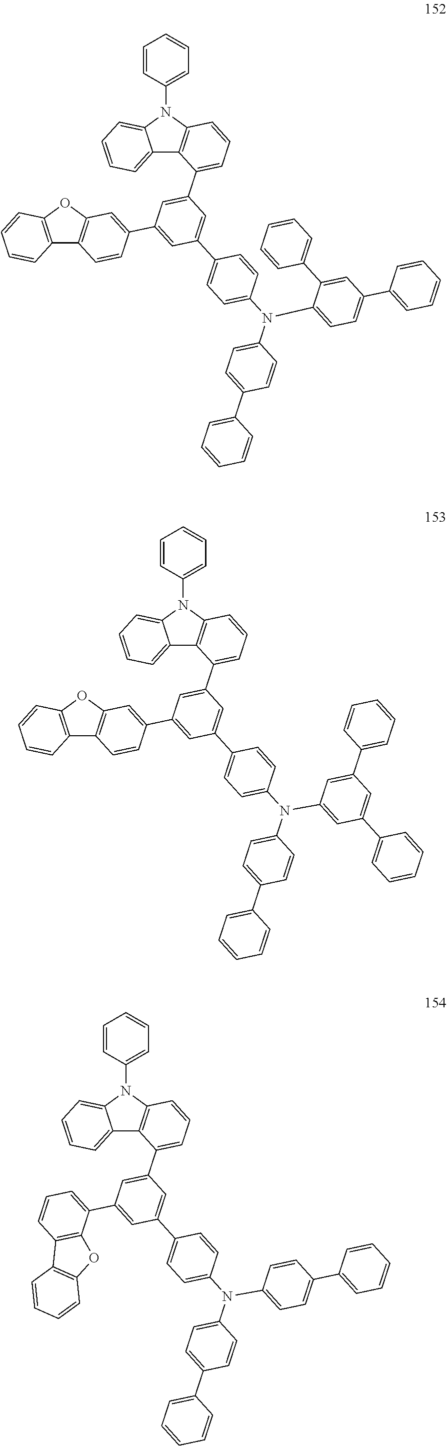 [SMILES: c1ccc(-c2ccc(N(c3ccc(-c4cc(-c5ccc6c(c5)oc5ccccc56)cc(-c5cccc6c5c5ccccc5n6-c5ccccc5)c4)cc3)c3ccc(-c4ccccc4)cc3-c3ccccc3)cc2)cc1 ; c1ccc(-c2ccc(N(c3ccc(-c4cc(-c5ccc6c(c5)oc5ccccc56)cc(-c5cccc6c5c5ccccc5n6-c5ccccc5)c4)cc3)c3cc(-c4ccccc4)cc(-c4ccccc4)c3)cc2)cc1 ; c1ccc(-c2ccc(N(c3ccc(-c4ccccc4)cc3)c3ccc(-c4cc(-c5cccc6c5oc5ccccc56)cc(-c5cccc6c5c5ccccc5n6-c5ccccc5)c4)cc3)cc2)cc1]

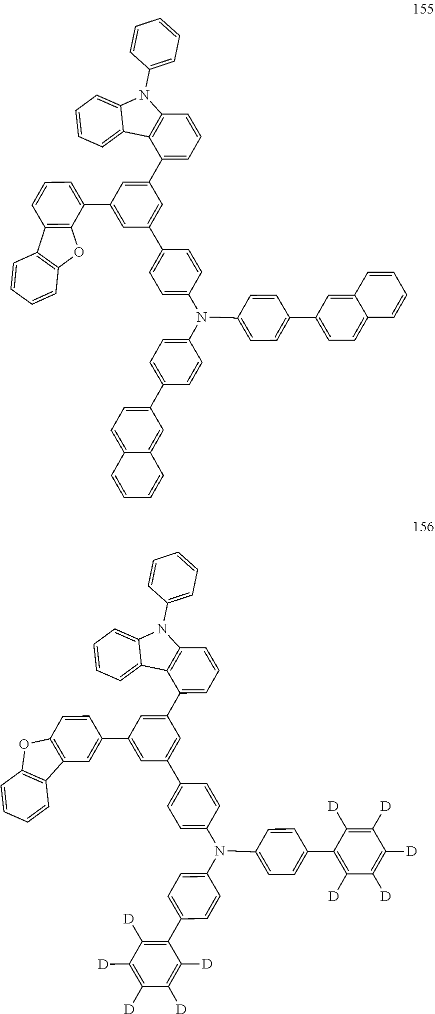 [SMILES: c1ccc(-n2c3ccccc3c3c(-c4cc(-c5ccc(N(c6ccc(-c7ccc8ccccc8c7)cc6)c6ccc(-c7ccc8ccccc8c7)cc6)cc5)cc(-c5cccc6c5oc5ccccc56)c4)cccc32)cc1 ; [2H]c1c([2H])c([2H])c(-c2ccc(N(c3ccc(-c4cc(-c5ccc6oc7ccccc7c6c5)cc(-c5cccc6c5c5ccccc5n6-c5ccccc5)c4)cc3)c3ccc(-c4c([2H])c([2H])c([2H])c([2H])c4[2H])cc3)cc2)c([2H])c1[2H]]

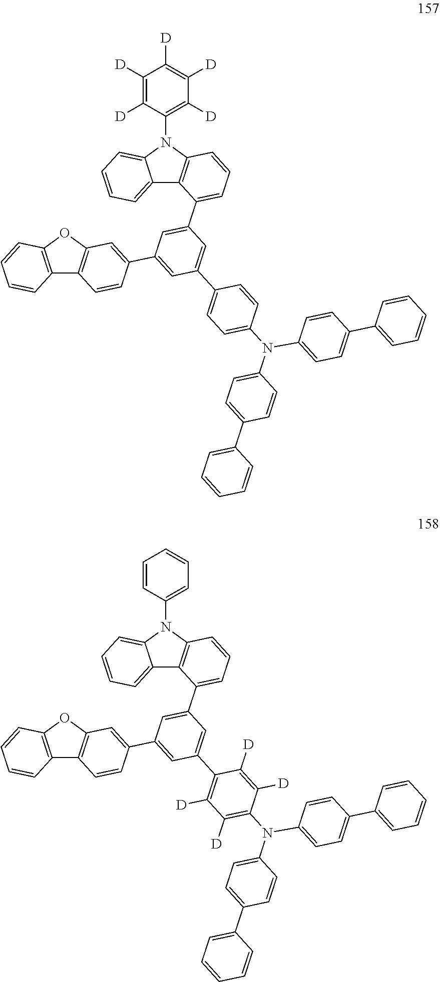 [SMILES: [2H]c1c([2H])c([2H])c(-n2c3ccccc3c3c(-c4cc(-c5ccc(N(c6ccc(-c7ccccc7)cc6)c6ccc(-c7ccccc7)cc6)cc5)cc(-c5ccc6c(c5)oc5ccccc56)c4)cccc32)c([2H])c1[2H] ; [2H]c1c([2H])c(N(c2ccc(-c3ccccc3)cc2)c2ccc(-c3ccccc3)cc2)c([2H])c([2H])c1-c1cc(-c2ccc3c(c2)oc2ccccc23)cc(-c2cccc3c2c2ccccc2n3-c2ccccc2)c1]

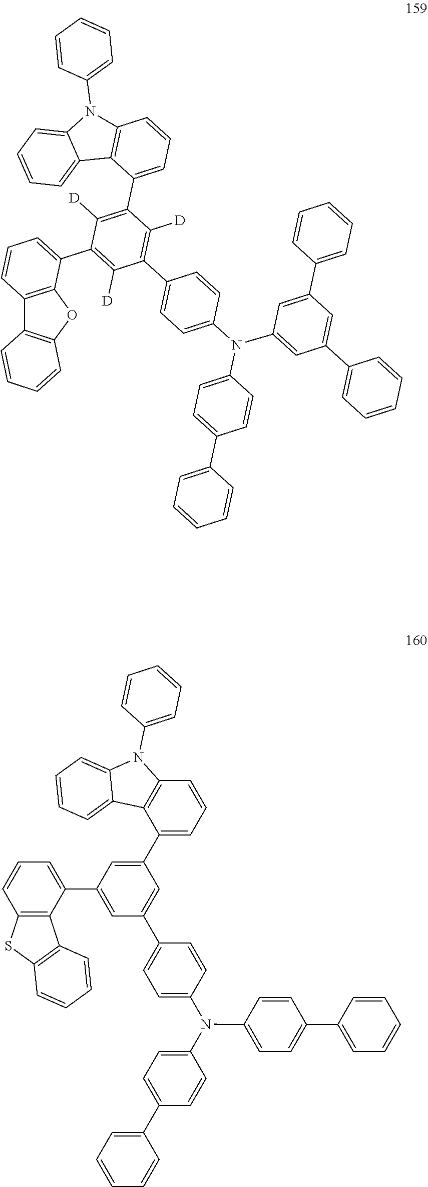 [SMILES: [2H]c1c(-c2ccc(N(c3ccc(-c4ccccc4)cc3)c3cc(-c4ccccc4)cc(-c4ccccc4)c3)cc2)c([2H])c(-c2cccc3c2c2ccccc2n3-c2ccccc2)c([2H])c1-c1cccc2c1oc1ccccc12 ; c1ccc(-c2ccc(N(c3ccc(-c4ccccc4)cc3)c3ccc(-c4cc(-c5cccc6sc7ccccc7c56)cc(-c5cccc6c5c5ccccc5n6-c5ccccc5)c4)cc3)cc2)cc1]

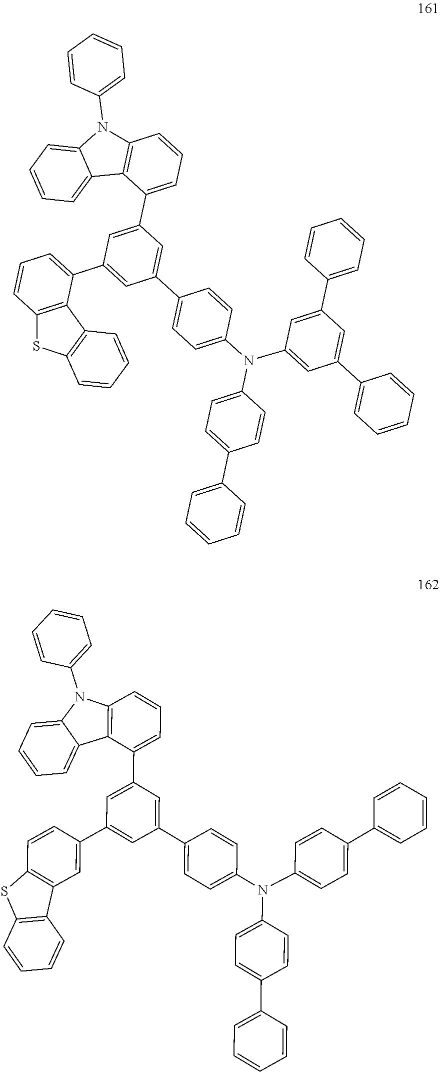 [SMILES: c1ccc(-c2ccc(N(c3ccc(-c4cc(-c5cccc6sc7ccccc7c56)cc(-c5cccc6c5c5ccccc5n6-c5ccccc5)c4)cc3)c3cc(-c4ccccc4)cc(-c4ccccc4)c3)cc2)cc1 ; c1ccc(-c2ccc(N(c3ccc(-c4ccccc4)cc3)c3ccc(-c4cc(-c5ccc6sc7ccccc7c6c5)cc(-c5cccc6c5c5ccccc5n6-c5ccccc5)c4)cc3)cc2)cc1]

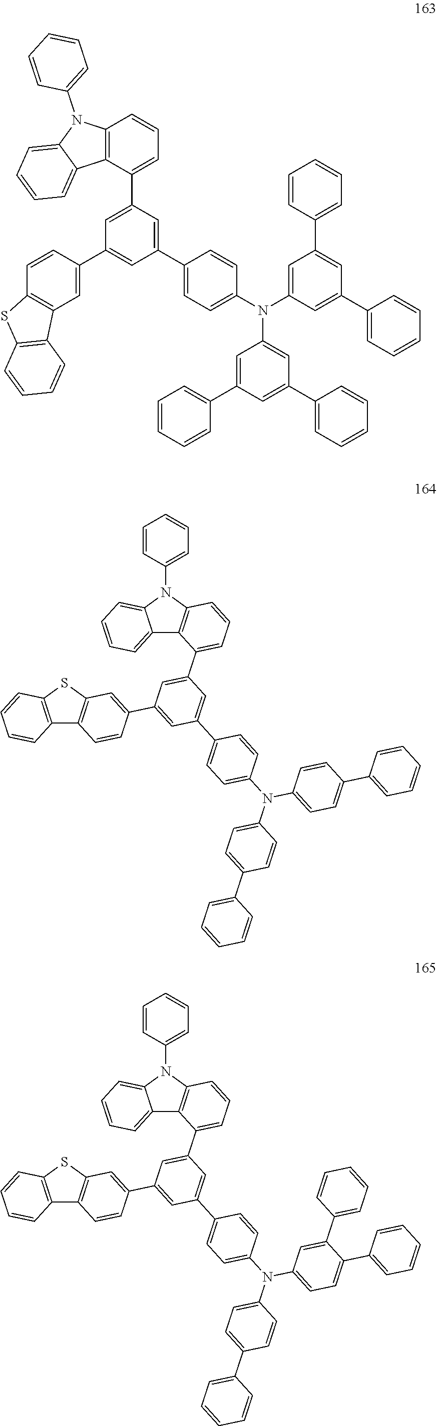 [SMILES: c1ccc(-c2cc(-c3ccccc3)cc(N(c3ccc(-c4cc(-c5ccc6sc7ccccc7c6c5)cc(-c5cccc6c5c5ccccc5n6-c5ccccc5)c4)cc3)c3cc(-c4ccccc4)cc(-c4ccccc4)c3)c2)cc1 ; c1ccc(-c2ccc(N(c3ccc(-c4ccccc4)cc3)c3ccc(-c4cc(-c5ccc6c(c5)sc5ccccc56)cc(-c5cccc6c5c5ccccc5n6-c5ccccc5)c4)cc3)cc2)cc1 ; c1ccc(-c2ccc(N(c3ccc(-c4cc(-c5ccc6c(c5)sc5ccccc56)cc(-c5cccc6c5c5ccccc5n6-c5ccccc5)c4)cc3)c3ccc(-c4ccccc4)c(-c4ccccc4)c3)cc2)cc1]

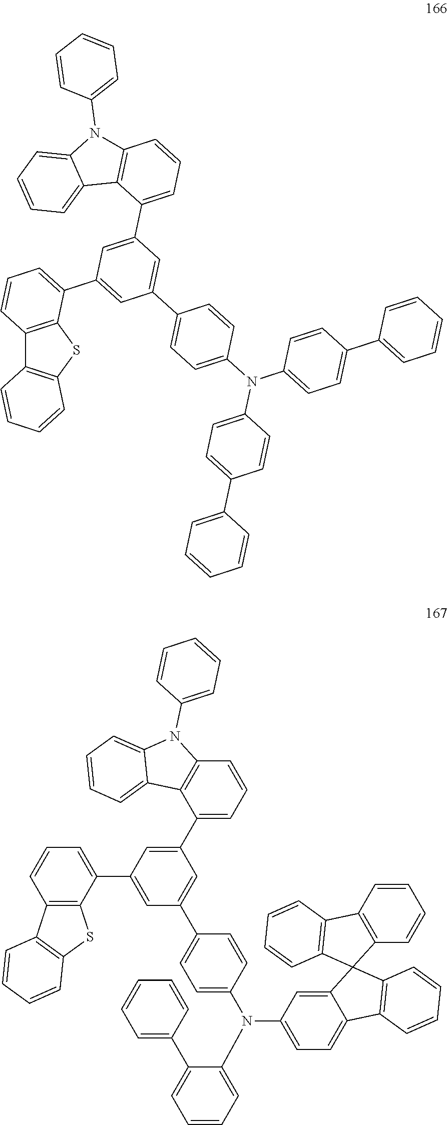 [SMILES: c1ccc(-c2ccc(N(c3ccc(-c4ccccc4)cc3)c3ccc(-c4cc(-c5cccc6c5sc5ccccc56)cc(-c5cccc6c5c5ccccc5n6-c5ccccc5)c4)cc3)cc2)cc1 ; c1ccc(-c2ccccc2N(c2ccc(-c3cc(-c4cccc5c4sc4ccccc45)cc(-c4cccc5c4c4ccccc4n5-c4ccccc4)c3)cc2)c2ccc3c(c2)C2(c4ccccc4-c4ccccc42)c2ccccc2-3)cc1]

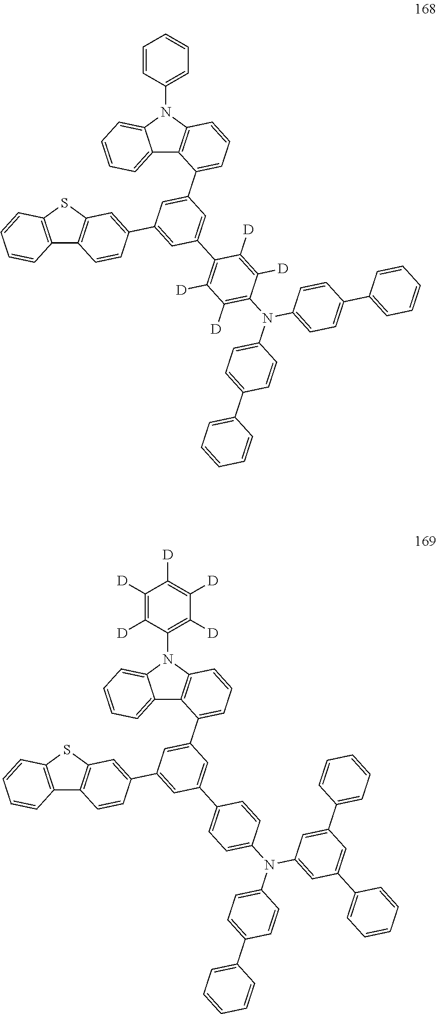 [SMILES: [2H]c1c([2H])c(N(c2ccc(-c3ccccc3)cc2)c2ccc(-c3ccccc3)cc2)c([2H])c([2H])c1-c1cc(-c2ccc3c(c2)sc2ccccc23)cc(-c2cccc3c2c2ccccc2n3-c2ccccc2)c1 ; [2H]c1c([2H])c([2H])c(-n2c3ccccc3c3c(-c4cc(-c5ccc(N(c6ccc(-c7ccccc7)cc6)c6cc(-c7ccccc7)cc(-c7ccccc7)c6)cc5)cc(-c5ccc6c(c5)sc5ccccc56)c4)cccc32)c([2H])c1[2H]]

7. An organic light-emitting device comprising a first electrode, a second electrode, and an organic layer having one or more layers disposed between the first electrode and the second electrode,
wherein one or more layers of the organic layer comprise one or more of the organic compounds implemented by [Formula I] according to claim 1.

8. The organic light-emitting device of claim 7, wherein the organic layer comprises one or more layers of an electron injecting layer, an electron transport layer, a hole injecting layer, a hole transport layer, an electron blocking layer, a hole blocking layer, and a light-emitting layer, and
one or more layers of the layers comprise the organic compound represented by [Formula I] above.

9. The organic light-emitting device of claim 8, wherein the electron blocking layer comprises the organic compound represented by [Formula I] above.
